SEQUENCE LISTING (7 sequences)

SEQ ID NO: 1 - protein, 163 aa, Artificial Sequence
  Synthetic Construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 112 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 106 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHM

SEQ ID NO: 4 - protein, 95 aa, Artificial Sequence
  Synthetic Construct
NQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 89 aa, Artificial Sequence
  Synthetic Construct
NQLYNELNLGRREEYDVLDKRRGRDPEMGGKPRRKNPQEGLYNELQKDKMAEAYSEIGMKGERRRGKGHDGLYQGLSTATKDTYDALHM

SEQ ID NO: 6 - protein, 112 aa, Artificial Sequence; identical to SEQ ID NO: 2
  Synthetic Construct

SEQ ID NO: 7 - protein, 112 aa, Artificial Sequence; identical to SEQ ID NO: 2
  Synthetic Construct

(19) United States
(12) Patent Application Publication
(10) Pub. No.: US 2020/0330515 A1
(43) Pub. Date: Oct. 22, 2020

(54) METHODS AND COMPOSITIONS RELATING TO ENGINEERED REGULATORY T CELLS

(71) Applicant: The General Hospital Corporation, Boston, MA (US)

(72) Inventors: Marcela V. MAUS, Lexington, MA (US); Angela BOROUGHS, Medford, MA (US)

(21) Appl. No.: 16/755,624

(22) PCT Filed: Oct. 3, 2018
(86) PCT No.: PCT/US2018/054180

§ 371 Date: Apr. 13, 2020

Related U.S. Application Data
(60) Provisional application No. 62/573,242, filed on Oct. 17, 2017.

(51) Int. Cl. A61K 35/17; C07K 16/28; C07K 14/725

(52) U.S. Cl. CPC A61K 35/17; C07K 16/28; C07K 2319/02; C07K 2319/03; C07K 14/7051

(57) ABSTRACT

The invention provides herein engineered regulatory T cells (Tregs), compositions thereof, and their methods of use. The Tregs described herein can be engineered to include, e.g., a chimeric antigen receptor (CAR), conferring increased stability, specificity, and immunosuppressive activity towards a target antigen. Furthermore, Tregs can also be engineered to reduce T cell cytotoxicity functions. Also provided are methods of suppressing immune response against a specific target antigen using the engineered Tregs described herein.

### CROSS-REFERENCE TO RELATED APPLICATIONS

This application claims priority to U.S. Provisional Application No. 62/573,242, filed on Oct. 17, 2017, which is incorporated herein by reference in its entirety.

### STATEMENT AS TO FEDERALLY FUNDED RESEARCH

This invention was made with government support under Grant No. NIH 5T32AI118692-02 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 1, 2018, is named 51295-011WO2_Sequence_Listing_10.1.18_ST25 and is 7,798 bytes in size.

### TECHNICAL FIELD

The technology described herein relates to engineered T cells, e.g., regulatory T cells (Tregs) engineered to recognize and/or localize to target cells of interest, e.g., for immunosuppression.

### BACKGROUND OF THE INVENTION

Regulatory T cells (Tregs) act as negative regulators of the cytotoxicity and proliferation of conventional T cells, are key modulators of inflammation, and are important for peripheral tolerance. While Tregs may impair anti-tumor immunity, a lack of Treg activity can result in autoimmune conditions or accelerate allograft organ or hematopoietic stem cell transplant (HSCT) rejection. Adoptive transfer of Tregs in preclinical mouse models has demonstrated therapeutic potential in solid organ transplantation (Nadig et al., Nat Med. 2010; 16(7):809-813), graft versus host disease (GvHD) (Edinger et al., Nat Med. 2003; 9(9):1144-1150), and a range of autoimmune diseases (Kohm et al., J Immunol. 2002; 169(9):4712-4716). Clinical trials using infusions of polyclonal Tregs cells are in development, but have shown modest efficacy in these diseases, with the most advanced data occurring in the setting of GvHD (Gliwinski et al., BioDrugs. 2017; 31(4):335-347). A challenge in the field of adoptive cell therapy has been generating Treg persistence after adoptive transfer. Previously, adoptive transfer of polyclonal Tregs in clinical trials required high doses of Tregs, which sometimes has to be given repetitively to have a clinical effect. Current hypotheses for these limitations have included the lack of antigen specificity to enable trafficking to the target organ, insufficient total numbers of Tregs to achieve adequate suppression, and poor persistence of the infused Tregs (Hoeppli et al., HLA. 2016; 88(1-2):3-13; Dawson et al., Front. Immunol. 2017; 8:1460; Wright et al., Proc. Natl. Acad. Sci. USA. 2009; 106(45):19078-19083). Accordingly, there exists a need for improved Treg therapies.

### SUMMARY OF THE INVENTION

Provided herein are engineered regulatory T cells (Tregs) and methods relating thereto that provide Treg cells with extended half-lives, the ability to localize to desired areas of a subject's body, and in some embodiments, the ability to localize to the immune cells causing the condition in need of treatment.

In one aspect, the invention provides a method of providing immunosuppression in a solid tissue in a subject, the method including administering to the subject an engineered regulatory T (Treg) cell including a chimeric antigen receptor (CAR), wherein the CAR includes: (i) an extracellular domain including an antigen-binding domain, (ii) a transmembrane domain, (iii) a signaling domain, and (iv) a co-stimulatory domain.

In some embodiments, the antigen-binding domain binds to an antigen expressed on the solid tissue, e.g., a skin cell. In some embodiments, the antigen is epidermal growth factor receptor (EGFR).

In some embodiments, the signaling domain is a CD3ζ signaling domain. In some embodiments, the co-stimulatory domain is a CD28 co-stimulatory domain.

In particular embodiments, the solid tissue is skin.

In one aspect of any of the embodiments, described herein is an engineered Treg cell comprising: a chimeric antigen receptor; and/or a nucleic acid encoding said chimeric antigen receptor.

In one aspect of any of the embodiments, described herein is a method of treating or preventing an autoimmune condition or allograft rejection in a subject in need thereof, the method comprising administering an engineered Treg cell as described herein to the subject.

In one aspect of any of the embodiments, described herein is a method of treating or preventing an autoimmune condition or allograft rejection in a subject in need thereof, the method comprising:
a. engineering a Treg cell to express at least a first chimeric antigen receptor;
b. administering the engineered Treg cell to the subject.

In one aspect of any of the embodiments, described herein is a method of treating or preventing an autoimmune condition or allograft rejection in a subject in need thereof, the method comprising:
a. engineering a Treg cell to express at least a first chimeric antigen receptor;
b. stimulating the Treg cell resulting from step a); and
c. administering the stimulated Treg cell to the subject.

In some embodiments of any of the aspects, simulating comprises contacting the cell with CD3 and/or CD28. In some embodiments of any of the aspects, the autoimmune condition is diabetes, neurologic disease, or graft-vs-host disease. In some embodiments of any of the aspects, a therapeutically effective amount of the cells are administered to the subject.

In some embodiments of any of the aspects, the chimeric antigen receptor comprises an extracellular domain that specifically binds to a first target molecule expressed on the surface of a first target cell. In some embodiments of any of the aspects, the first target cell is a cell affected by an autoimmune condition and/or allograft rejection. In some embodiments of any of the aspects, the cell further comprises a second chimeric antigen receptor; and/or a nucleic acid encoding said second chimeric antigen receptor, wherein the second chimeric antigen receptor comprises an extracellular domain that specifically binds a different target molecule than the first chimeric antigen receptor. In some embodiments of any of the aspects, wherein the second chimeric antigen receptor comprises an extracellular domain that specifically binds to a second target molecule expressed on the surface of a second target cell. In some embodiments of any of the aspects, the second target cell is an immune system cell contributing to an autoimmune condition and/or allograft rejection. In some embodiments of any of the aspects, the second target cell is a Treg cell. In some embodiments of any of the aspects, the second target molecule is selected from the group consisting of: CTLA4; CD25; CD27; PDL1; GARP; TGFbeta; and LAP. In some embodiments of any of the aspects, the second target cell is a myeloid derived suppressor cell (MDSC). In some embodiments of any of the aspects, the second target molecule is selected from the group consisting of: CD32; CD33, and CD11c.

In some embodiments of any of the aspects, a chimeric antigen receptor comprises:
i. an extracellular target-binding domain;
ii. a hinge/transmembrane domain; and
iii. an intracellular co-stimulation domain.

In some embodiments of any of the aspects, a chimeric antigen receptor comprises:
i. an extracellular target-binding domain;
ii. a hinge/transmembrane domain;
iii. an intracellular co-stimulation domain; and
iv. a primary signaling domain.

In some embodiments of any of the aspects, a chimeric antigen receptor comprises:
i. an extracellular target-binding domain;
ii. a hinge/transmembrane domain; and
iii. a primary signaling domain.

In some embodiments of any of the aspects, a primary signaling domain is or comprises a CD3 z chain. In some embodiments of any of the aspects, the chimeric antigen receptor further comprises an N-terminal leader sequence.

In some embodiments of any of the aspects, a Treg cell is a CD8− CD4+ CD25+ CD127+ cell. In some embodiments of any of the aspects, a Treg cell is a CD8− CD4dim CD25 hi and CD127 low cell. In some embodiments of any of the aspects, the Treg cell is a T cell expressing one or more markers selected from the group consisting of: CTLA4; PDL1; LAP; GARP; CD25; and CD27.

In some embodiments of any of the aspects, the leader sequence is a CD8 leader sequence. In some embodiments of any of the aspects, the hinge/transmembrane domain is a CD8 hinge/transmembrane domain. In some embodiments of any of the aspects, the intracellular co-stimulation domain is a 4-1BB intracellular co-stimulation domain. In some embodiments of any of the aspects, the intracellular co-stimulation domain is a CD28 intracellular co-stimulation domain. In some embodiments of any of the aspects, the target-binding domain is an antibody reagent. In some embodiments of any of the aspects, the target-binding domain is an scFv. In some embodiments of any of the aspects, the first target molecule is CD19.

In some embodiments of any of the aspects, the engineered cell is a mammalian cell. In some embodiments of any of the aspects, the engineered cell is a human cell. In some embodiments of any of the aspects, the engineered cell is a murine cell. In some embodiments of any of the aspects, the engineered cell is autologous to a subject. In some embodiments of any of the aspects, the engineered cell is allogeneic to a subject. In some embodiments of any of the aspects, the engineered cell is further engineered to reduce expression of an endogenous T cell receptor and/or an endogenous MHC complex.

In some embodiments of any of the aspects, the engineered cell further comprises:
a. exogenous FoxP3; CTLA4; PDL1; and/or TGFβ polypeptides; and/or
b. an exogenous nucleic acid encoding FoxP3; CTLA4; PDL1; and/or TGFβ polypeptides.

In some embodiments of any of the aspects, the engineered cell is further engineered to not comprise a T cell cytotoxicity functions. In some embodiments of any of the aspects, the engineered cell is further engineered to not express a functional perforin, granzyme, and/or Fas-ligand gene.

In another aspect, the invention provides an engineered regulatory T cell (Treg) with reduced T cell cytotoxicity function.

In some embodiments, perforin, granzyme, and/or Fas-ligand gene expression is reduced.

In some embodiments, the Treg includes a chimeric antigen receptor (CAR) and/or a nucleic acid capable of encoding the CAR.

In some embodiments, the CAR includes (i) an extracellular domain including an antigen-binding sequence, (ii) a transmembrane domain, and (iii) a T cell intracellular signaling domain. In other embodiments, the CAR further includes (iv) one or more co-stimulatory domains.

In some embodiments, the CAR further includes an N-terminal leader sequence, e.g., CD8 leader sequence. In some embodiments, the antigen-binding sequence is an antibody reagent, e.g., an scFv. In other embodiments, the CAR further includes a hinge domain selected from the group consisting of the hinge domains of CD8, CD4, CD28 and CD7. In particular embodiments, the hinge domain is a CD8 hinge domain. In further embodiments, the transmembrane domain is selected from the group consisting of the transmembrane domains of the alpha, beta, and zeta chains of the T-cell receptor, CD3ε, CD3ζ, CD4, CD5, CD8, CD9, CD16, CD22, CD27, CD28, CD33, CD37, CD45, CD64, CD80, CD86, CD134 (OX40), CD137 (4-1BB), CD152 (CTLA4), CD154, and PD-1. In specific embodiments, the transmembrane domain is a CD8 transmembrane domain.

In some embodiments, the T cell intracellular signaling domain is selected from the group consisting of the intracellular signaling domains of TCRζ, FcRγ, FcRβ, CD3γ, CD3δ, CD3ε, CD3ζ, CD22, CD79a, CD79b, and CD66d. In particular embodiments, the T cell intracellular signaling domain is a CD3ζ intracellular signaling domain.

In some embodiments, the co-stimulatory domain is selected from the group consisting of the co-stimulatory domains of CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In particular embodiments, the co-stimulatory domain is a 4-1BB co-stimulatory domain. Alternatively, the co-stimulatory domain is a CD28 co-stimulatory domain.

In some embodiments, the antigen-binding sequence is specific to an antigen expressed by a cell affected by a disease or disorder, e.g., an autoimmune disease and/or an allograft rejection.

In some embodiments, the Treg further includes a second CAR, and/or a nucleic acid encoding a second CAR, including (i) a second extracellular domain including a second antigen-binding sequence, (ii) a second transmembrane domain, and (iii) a second T cell intracellular signaling domain, and wherein the second antigen-binding sequence is specific to a second antigen different from the first antigen-binding sequence. In some embodiments, the second CAR further includes (iv) one or more co-stimulatory domains.

In some embodiments, the second antigen is expressed by an immune cell, e.g., a Treg or a myeloid derived suppressor cell (MDSC) contributing to an autoimmune disease. In some embodiments, the second antigen is selected from the group consisting of CTLA4, CD25, CD27, PD-L1, GARP, TGFβ, and LAP. In other embodiments, the second antigen is selected from the group consisting of CD32, CD33, and CD11c.

In some embodiments of any of the preceding aspects, the Treg is a mammalian cell, e.g., a human cell or a murine cell. In some embodiments, the Treg is autologous to a subject.

Alternatively, the Treg is allogeneic to a subject.

In some embodiments, the Treg is further engineered to reduce expression of an endogenous T cell receptor and/or an endogenous MHC complex.

In other embodiments, the Treg further includes a) exogenous Foxp3, CTLA4, PD-L1, and/or TGFβ polypeptides; and/or b) an exogenous nucleic acid encoding Foxp3, CTLA4, PD-L1, and/or TGFβ polypeptides.

In some embodiments, the Treg is a CD8−, CD4+, CD25+, and CD127+ cell. Alternatively, the Treg is a CD8−, CD4dim, CD25hi, and CD127low cell. The Treg may express one or more markers selected from the group consisting of CTLA4, PD-L1, LAP, GARP, CD25, and CD27.

In another aspect, the invention provides a pharmaceutical composition including the Treg any of the preceding embodiments and a pharmaceutically acceptable carrier.

In another aspect, the invention provides a method of treating or preventing an autoimmune disease and/or an allograft rejection in a subject in need thereof, the method including administering the Treg or the pharmaceutical composition of any of the preceding aspects to the subject.

In another aspect, the invention provides a method of treating or preventing an autoimmune disease and/or an allograft rejection in a subject in need thereof, the method including (a) engineering a Treg to reduce T cell cytotoxicity functions, and (b) administering the engineered Treg to the subject.

In another aspect, the invention provides a method of treating or preventing an autoimmune disease and/or an allograft rejection in a subject in need thereof, the method including (a) engineering a Treg to reduce T cell cytotoxicity functions, wherein the Treg includes a CAR and/or a nucleic acid capable of encoding said CAR, and (b) administering the engineered Treg to the subject.

In another aspect, the invention provides a method of treating or preventing an autoimmune disease and/or an allograft rejection in a subject in need thereof, the method including (a) engineering a Treg to reduce T cell cytotoxicity functions, wherein the Treg includes a CAR and/or a nucleic acid capable of encoding said CAR, (b) stimulating (e.g., by contacting the cell with CD3 and/or CD28) the Treg of step (a), and (c) administering the stimulated Treg to the subject.

In some embodiments of any of the preceding aspects, the Treg is engineered to reduce perforin, granzyme, and/or Fas-ligand gene expression.

In some embodiments of any of the preceding aspects, the CAR is specific to an antigen expressed by a cell affected by the autoimmune disease and/or allograft rejection.

In some embodiments of any of the preceding aspects, the autoimmune disease is diabetes, neurologic disease, or graft versus host disease.

In some embodiments of any of the preceding aspects, a therapeutically effective amount of Tregs is administered to a subject.

### BRIEF DESCRIPTION OF THE DRAWINGS

FIGS. 1A-1D show CD4+ T cells isolated from human donor, MACs selected for PE-CD25+ cells, and sorted. FIG. 1A shows the results of CD4dim, CD25hi and CD127low. FIG. 1B shows the results of CD4+ CD25 low. Gates drawn on not enriched cells. FIG. 1C depicts a graph of Foxp3 (clone PCH101) intracellular stain after sort. FIG. 1D depicts a graph of methylation of Treg Specific Demethylation Region (TSDR) after sort in female donors using direct bisulfite Modification and Pyrosequencing by EpigenDx N=2.

FIGS. 2A-2D show in vitro experimental design. FIG. 2A shows a time line of Treg and T cony cell transduction, expansion followed by 7 day resting period before assays outlined in FIG. 2B. FIG. 2B shows assays used to test Treg function. FIG. 2C shows BBζT cony cell and FIG. 2D shows BBζ Treg transduction efficiency as measured by mCherry expression. Tregs and Tconv were cultured in OpTmizer™ media with 2% human male serum and IL-2 (300 units/ml and 20 units/ml respectively). Cells were kept at 1 million cells/ml and media and IL-2 were added every 2 to 3 days.

FIG. 3 shows schematics of CARs were designed with a humanized scFv to CD19 (clone: WO 2014/153270 A1) in a pMGH lentiviral backbone. Constructs have a CD8 leader and hinge/transmembrane region followed by co-stimulation (CD28 or 4-1BB) and a CD3ζ chain. CAR Δζ contains only a truncated CD3ζ. mCherry was added after a T2A sequence. In CAR 28ζ Foxp3 construct, a Foxp3 transgene was added after a second T2A. Lentivirus was made and T cells were transduced day 1 after CD3/CD28 bead stimulation with an MOI of 5. Transduction efficiency was ˜80%.

FIGS. 4A-4B show Tregs surface markers. Day (D) 1, D14 and D23, Tregs and CD4+ Tconv cells were surface stained for LAP-PeCy7, LAG3-PercpCy5.5, CD39-FITC and CTLA4-APC. FIG. 4A shows surface markers before stimulation and CAR transduction. FIG. 4B shows the gating strategy for surface flow cytometry.

FIGS. 5A-5E show that Foxp3 expression is stable after transduction and bead expansion. The type of co-stimulation does not affect Treg stability by Foxp3 flow cytometry and TSDR demethylation. FIG. 5A shows a timeline showing stimulations and time points for flow and TSDR measurements (represented with a star). FIG. 5B shows representative male donor TSDR methylation D14 and D23 after CD19 stimulation. FIG. 5C shows a graph of TSDR methylation of CAR Treg normalized to untransduced Tregs n=3 human donors. Tregs were stained with CD3, CD8, CD4, CD25 and then fixed and permabilized with Foxp3 intracellular staining kit (eBioscience®) then strained with Foxp3-APC PCH101. FIG. 5D depicts a graph of Foxp3+ CD25+ of CD3+ CD4+ live cells. FIG. 5E depicts a graph of Foxp3 MFI of CD3+ CD4+ live cells. N=5.

FIGS. 6A-6B show representative flow plots showing gating for CD25+ Foxp3+ intracellular stain.

FIGS. 7A-7B show that surface markers of CAR Tregs increase upon CAR T cell activation. Depicted are graphs of surface expression by flow cytometry of (FIG. 7A) CTLA4 as a % of CD4+ mCherry+ cells and (FIG. 7B) log fold change of % of CTLA4+ cells compared to Δζ CAR Treg with CD3 stim as a way to normalize across donors. Mean and SEM plotted.

FIGS. 8A-8B show that surface markers of CAR Tregs increase upon CAR T cell activation. Depicted are graphs of surface expression by flow cytometry of (FIG. 8A) CD39 as a % of CD4+ mCherry+ cells and (FIG. 8B) log fold change of % of CD39+ cells compared to Δζ CAR Treg with CD3 stim as a way to normalize across donors. Mean and SEM plotted.

FIGS. 9A-9B show that surface markers of CAR Tregs increase upon CAR T cell activation. Depicted are graphs of surface expression by flow cytometry of (FIG. 9A) LAP as a % of CD4+ mCherry+ cells and (FIG. 9B) log fold change of % of LAP+ cells compared to delZ CAR Treg with CD3 stim as a way to normalize across donors. Mean and SEM plotted.

FIGS. 10A-10B show that surface markers of CAR Tregs increase upon CAR T cell activation. Depicted are graphs of surface expression by flow cytometry of (FIG. 10A) LAP as a % of CD4+ mCherry+ cells and (FIG. 10B) Log fold change of % of LAP+ cells compared to delZ CAR Treg with CD3 stim as a way to normalize across donors. Mean and SEM plotted.

FIG. 11 shows a graph of surface expression by flow cytometry of LAGS as a % positive of CD4+ mCherry+ cells. Mean and SEM plotted.

FIGS. 12A-12B show that Tregs produce less inflammatory cytokines and high IL-10. FIG. 12A shows IL-2 and IFNγ levels measured by flow cytometry in Tconv and Tregs after Nalm6 stimulation. FIG. 12B shows IFNγ, IL-2, TNFα, and IL-10 levels measured in parallel by Luminex®. Cells were washed and plated in OpTimizer™ with 100,000 CAR T cells/well stimulated with K562-CD19 or K562-anti-CD3 at a 1:1 ratio for 20 hours and then the supernatants were frozen at −80° C. Cytokines were measured in 50 μL of supernatants using Luminex® assay Th1/Th2 cytokine panel 11plex and IL-10 kit. N=3 different human donors.

FIG. 13 shows a graph demonstrating that CAR stimulation increases expression of IL-6 in Tregs.

FIGS. 14A-14C show that CAR Tregs proliferate in response to antigen stimulation. Tregs were violet labeled and plated at 50,000/well in a 96 well plate with a 1:1 ration of irradiated Nalm6 or no stimulation in R10 and 300 units/mL IL-2. FIG. 14A show that violet labeled Tregs proliferate with irradiated Nalm6 cells at 1:1 ratio over 3 days. After bead expansion and one week of rest, T cells were stimulated with K562 CD19 (FIG. 14B) or anti-CD3 (FIG. 14C) cell expansion and fold change were measured by cell counting.

FIGS. 15A-15F show that CAR Tregs cells suppress CAR T cell proliferation and cytokine secretion. CAR T conventional cells were CFSE labeled and CAR Tregs were violet labeled. Cells were mixed at appropriated ratios starting with 50,000 CAR T cells per well in triplicate in R10 and 100,000 irradiated Nalm6 cells were added to each well. After 3 days, plates were stained with CD3 and live/dead and collected by flow cytometry. Cells were gated on CD3+ live mCherry+ and CFSE+ and then CFSE dilution was measured. FIG. 15A shows representative single donor inhibition of proliferation assay. FIG. 15B shows inhibition of proliferation assay with n=5 donors. Supernatants were collected in the same assay set up after 24 hours and cytokines were measured by Luminex®. Graphs depict suppression of IL-2 (FIG. 15C), GM-CSF (FIG. 15D), IFNγ (FIG. 15E) and TNFα (FIG. 15F) by T cony 28ζ CARs. Note: all cells were at 50% mCherry+ for assay (diluted with untranduced cells). Representative donor. N=3

FIGS. 16A-16D show that stable Foxp3+ Tregs still kill target cells. FIG. 16A shows luciferase based killing assay using Nalm 6 CBG-GFP cells and shows that Tregs kill target cells. N=3. FIG. 16B shows a degranulation assay using live Nalm 6 cells in R10 with CD107a-AF700 at 2 μl/100 μl incubated at 37° C. for 6 and 2 hours. Tregs degranulated upon antigen stimulation. N=3. FIG. 16C shows that the expression of transgenic Foxp3 did not prevent this degranulation as showed by IC staining of Foxp3 after 6 hour incubation with nalm6 and CD107a. FIG. 16D shows that CAR Tregs (except delZ) upregulated CD8 on CD4+ T cells following activation.

FIG. 17 shows graphs of killing assays. N=3.

FIG. 18 shows graphs of degranulation assays. N=3.

FIG. 19 shows flow cytometry data from the degranulation assay showing surface CD107a and intracellular staining of Foxp3 representative figure. (N=3)

FIG. 20 shows that CD8 is upregulated in all CD4 CAR T cells but to a greater extent in the CAR Tregs.

FIG. 21 shows validation of the CD2K-Foxp3 construct.

FIG. 22A shows the sorting strategy for CD4+ T cells isolated from human donor PBMCs and enriched for CD25+ cells using positive selection. Sorting gates for Tregs: CD4^{mid}, CD25⁺⁺ and CD127^{low} and for Tconv: CD4⁺, CD25^{low}; gates drawn prior to enrichment.

FIG. 22B shows the Foxp3 (clone PCH101) intracellular stain after sort.

FIG. 22C shows intracellular staining of Foxp3 displayed as mean fluorescence intensity (MFI).

FIG. 22D shows methylation status across the Treg Specific Demethylation Region (TSDR) on T cell populations after fluorescence-activated cell sorting from female-donor leukopaks. N=2 female donors, mean and SEM plotted.

FIG. 22E shows surface staining of surface markers LAP, LAG, CD39 and CTLA4. N>3 human donors, mean and SEM plotted.

FIG. 23A shows vector maps of CD19 CAR constructs Δζ, 28ζ, BBζ, and 28ζ-Foxp3. TM refers to the hinge and transmembrane domain, L refers to leader sequence.

FIG. 23B shows the experimental design.

FIG. 23C shows the representative transduction efficiency of BBζ CAR constructs as determined by mCherry expression 13 days post-sort.

FIG. 23D depicts Foxp3 expression by intracellular staining and flow cytometry. Tconv cells and Tregs were transduced to express 28ζ or 28ζ-Foxp3 at an MOI of 5. Data shown from representative donor.

FIG. 24A shows the intracellular staining of Foxp3±CD25+ cells gated on CD3+, CD4+, live cells after sorting (D0), bead expansion and rest (D14), and on day 23, 9 days after the addition of irradiated anti-CD3 K562 (TCR Stim) or CD19-K562 (CAR Stim).

FIG. 24B shows Foxp3 intracellular staining by MFI of T cells gated on live CD3+CD4+ cells after sorting (D0), after bead expansion and rest (D14) and on day 23, 9 days after stimulation with irradiated anti-CD3 K562 (TCR Stim) or irradiated CD19-K562 stimulation (CAR Stim). N=6 human donors.

FIG. 24C shows the TSDR methylation day 0 post sort (D0), day 14, and day 23 (9 days after irradiated CD19-K562 stimulation). N=2 female donors.

FIG. 24D shows the methylation status of CTLA4 promotor using direct bisulfite modification and pyrosequencing. N=3 human donors.

FIG. 24E shows methylation status of the IKZF2 (Helios) promotor.

FIG. 24F shows methylation of TSDR from mCherry+ cells day 0 post sort (D0), day 14 and day 23 (9 days after irradiated CD19-K562 stimulation using direct bisulfite modification and pyrosequencing. N=3 human donors. Bars for D0 represent UT Tregs and UT Tconv directly after sort.

FIG. 25A shows surface CTLA4 9 days after TCR or CAR stimulation with irradiated K562 cells. N=3 human donors. Mean and SEM plotted.

FIGS. 25B-25C shows surface expression of T cells gated on of CD3⁺ CD4⁺ CAR⁺ stimulated with K562 cells for 9 days. FIG. 25B shows LAP staining and FIG. 25C shows CD39 staining measured by flow cytometry.

FIG. 26A shows surface staining of CD69 after cells were left unstimulated or stimulated with irradiated CD19-K562 or anti-CD3-K562 at a 1:1 ratio for 20 hours.

FIG. 26B shows surface staining of latent associated peptide (LAP) after cells were left unstimulated or stimulated with irradiated CD19-K562 or anti-CD3-K562 at a 1:1 ratio for 20 hours. LAP was measured as % positive after gating on live, mCherry⁺, CD4⁺ T cells except in the case of the UT groups which were only gated on live CD4⁺ cells.

FIGS. 26C-26D show 4-1BB (CD137) surface expression of live CD4⁺ CAR⁺ cells after cells were left unstimulated or stimulated with irradiated CD19-K562 or anti-CD3-K562.

FIG. 26C shows 4-1BB expression as a % and FIG. 26D shows 4-1BB expression as raw MFI. N=3 human donors. Mean and SEM plotted.

FIGS. 27A-27C show levels of various cytokines measured by Luminex® in the supernatants saved 20 hours after T cell-K562 co-culture. Cytokine levels were normalized to Tc UT stimulated with K562-OKT3. N=3 human donors, ** p<0.01 for CAR stimulated versus TCR stimulated comparisons. FIG. 27A shows the levels of IL-2. FIG. 27B shows the levels of TNFα. FIG. 27C shows the levels of IFNγ.

FIG. 27D shows the levels of IL-10 detected in the supernatants of T cell and CD19-K562 co-cultures. N=5 human donors, ** p<0.01 for paired t-test between BBζ and 28ζ Treg groups. Cytokine values were normalized to Tc UT stimulated with irradiated anti-CD3 K562 target cells. No cytokines were detected in the wells with K562 cells alone or in T cells with no targets conditions.

FIG. 28A shows T cell proliferation after T cells were stimulated on day 14 after bead expansion and one week of rest (Time-point 0) at a 1:1 ratio with irradiated K562s expressing CD19. Live cell numbers were counted every two days and expressed as the log 2 fold change from the starting cell number. Significance was calculated by a paired t-test at day 8 between Tr Δζ and Tr 28ζ. ** p<0.01. Mean and SEM plotted. Tc—Tconv cells, Tr—Treg, NS—not significant.

FIG. 28B shows T cell proliferation after T cells were stimulated on day 14 after bead expansion and one week of rest (Time-point 0) at a 1:1 ratio with irradiated K562s expressing anti-CD3. Live cell numbers were counted every two days and expressed as the log 2 fold change from the starting cell number. Significance was calculated by a paired t-test at day 8 between Tr Δζ and Tr 28ζ. ** p<0.01. Mean and SEM plotted. Tc—Tconv cells, Tr—Treg, NS—not significant.

FIG. 28C shows Treg proliferation after violet cell trace-labeling and activation with irradiated CD19-K562 cells over 3 days. The number of mCherry⁺ proliferating (violet low) cells was normalized to the number of mCherry⁺ violet low cells in the unstimulated condition. N=3 human donors. Mean and SEM plotted. p *<0.05 p **<0.01. paired t-test to Tr Δζ.

FIG. 29A shows 28ζ CAR-Tregs in MLR with CFSE labeled first- and second-generation CAR-Teff cells and irradiated Nalm6 target cells. Representative donor.

FIG. 29B shows T cell proliferation after MLRs of CFSE labeled CD19 CAR Teff cells with different ratios of violet-labeled CAR-Tregs. After 3 days, CFSE dilution was measured on mCherry⁺ Teff cells to calculate proliferation as a % of the number of mCherry⁺ Teff cells proliferating with no Tregs present. N=5 human donors. Mean and SEM plotted, p *<0.05, paired t-test performed for 1:1 Treg-to-Teff ratio only.

FIGS. 30A-30D show levels of various cytokines in the supernatants collected from the MLR after 24 hours. FIG. 30A shows the levels of TNFα. FIG. 30B shows the levels of GM-CSF. FIG. 30C shows the levels of IL-2. FIG. 30D shows the levels of IFNγ. Data is shown from a representative donor, repeated with N=3 donors with technical triplicates.

FIG. 31A shows Treg suppression of Teff cell proliferation after activation though the CAR (CFSE labeled CD19ζ CAR-Teff, irradiated Nalm6 targets, 1:2 Teff-to-target cell ratio). CSFE was measured after 4 days, and data is shown from a representative donor of N=3 with technical triplicates. All cells were at 50% mCherry+ for the assay (diluted with UT T cells).

FIG. 31B shows Treg suppression of Teff cell proliferation after activation though the TCR (CFSE labeled naïve T cells, anti-CD3/anti-CD28 beads, 10:1 cell-to-bead ratio). CSFE was measured after 4 days, and data is shown from a representative donor of N=3 with technical triplicates. All cells were at 50% mCherry+ for the assay (diluted with UT T cells).

FIG. 32A shows the MLR run with or without IL-10 blocking antibody at different ratios of CD19 28ζ CAR-Tregs to CD19 CAR Teff cells with irradiated Nalm6 cells as targets. Representative donor with technical triplicates.

FIG. 32B shows an IL-2 consumption assay. IL-2 cytokine levels after Tregs and Tconv cells expressing different CARs were incubated with the same initial concentrations of IL-2 for 40 hours either with or without the addition of irradiated Nalm6 cells. Representative donor with technical triplicates' mean and SEM plotted. N=4 human donors. p *<0.05 p **<0.01. Significant p values were found for each donor tested.

FIG. 33A shows T cells in culture flasks.

FIG. 33B shows representative images of CAR Tregs in culture resting. Images were taken with a 40× objective with bright light or an RFP detecting light cube.

FIGS. 34A and 34B show luciferase based killing assay using Nalm6 CBG-GFP. N=3 normal donors.

FIG. 34C shows degranulation assay of CAR T cells calculated as the percentage CD107a⁺ cells of total mCherry⁺ or mCherry⁻ cells per condition over 6 hour co-culture with live Nalm6 cells. N=3 human donors. Mean and SEM plotted. Data was from non-fixed cells.

FIGS. 34D and 34E show degranulation assay of CAR T cells calculated as the percentage CD107⁺ cells of CAR′ (mCherry⁺) or UT T cells per well. FIG. 34D shows percentage of CD107⁺ cells over 2 hours with PMA/ionomycin stimulation. FIG. 34E shows percentage of CD107⁺ cells over no stimulation. Tc—Tconv cells, Tr—Treg.

FIG. 34F shows representative donor flow plots of CD107a versus Foxp3 expression after a 6 hour degranulation assay with Nalm6. T cells were fixed, permeabilized and stained for intracellular Foxp3. Repeated with N=3 human donors.

FIG. 35A shows the vector map of EGFRvIII ζ CAR constructs. TM, hinge and transmembrane domain. L, leader sequence.

FIG. 35B shows mixed lymphocyte reaction (MLR) of CFSE labeled EGFRvIII CAR-Teff with different EGFRvIII ζ CAR Treg ratios stimulated with U87-EGFRvIII cells. After 4 days, CFSE dilution was measured to calculate % proliferation as the % CFSE low mCherry⁺ Teff cells compared to the % of CFSE low mCherry⁺ Teff cells with no Tregs present. Representative donor.

FIG. 35C shows luciferase-based killing assay using U87-EGFRvIII CBG-GFP cells incubated with ζ CAR-Tregs and ζ Tconv cells with CARs against CD19 or EGFRvIII at varying ratios for 16 hours. Representative donor.

FIG. 35D shows degranulation assay of CAR T cells in media with CD107a antibody and Befeldin A after 6 hour stimulation with U87-EGFRvIII target cells. Gated on CD3⁺ mCherry⁺ (CAR⁺) or mCherry⁻ (UT T cells) within a sample. Representative donor.

FIG. 35E shows degranulation assay of CAR T cells in media with CD107a antibody and Befeldin A after 6 hour stimulation with U87-CD19 target cells. Gated on CD3⁺ mCherry⁺ (CAR⁺) or mCherry⁻ (UT T cells) within a sample. Representative donor.

FIG. 35F shows luciferase-based assays using Tregs sorted on CD45RA⁺ (naïve) Treg cells and Nalm6-CPG-GFP Target cells. Representative donor with technical triplicates. Mean and SEM plotted. Tc—Tconv cells, Tr—Treg, TrN—Naïve Tregs

FIG. 36A shows GZMB expression of cDNA isolated from Tregs stimulated with Nalm6 for 24 hours and sorted on CD4⁺ mCherry⁺ cells. Gene expression was measured by digital droplet PCR and expressed as a relative ratio to internal control gene TBP. Mean and SEM plotted. N=3 human donors.

FIGS. 36B-36D show various gene expression levels from cDNA isolated from CD19 CAR-Tconv and CD19 CAR-Treg cells stimulated with Nalm6 for 24 hours and sorted on CD4⁺ mCherry⁺ cells. Gene expression was measured by digital droplet PCR and expressed as a relative ratio to TBP (T cell internal control gene). N=4 normal donors. FIG. 36B shows GZMB expression. FIG. 36C shows GZMA expression. FIG. 36D shows PRF1 expression.

FIG. 37A shows luciferase-based killing assays of Nalm6 CBG-GFP with granzyme/perforin inhibitors CMA or Z-AAD-CMK (1:3 Tcell-to-target ratio, 16-hour incubation time) added to media. Representative donor, mean and SEM of triplicates plotted. Repeated with N=3 human donors, *p<0.05 **p<0.01 ***p<0.001 paired t-test. Tc—Tconv cells, Tr—Treg.

FIG. 37B shows luciferase-based killing assays of Nalm6 cells were repeated with CD19 28ζ-Tconv or UT-Tconv plus granzyme/perforin inhibitors CMA and Z-AAD-CMK (1:3 Tconv to target ratio, 16-hour incubation time). UT-Tconv cells shown as the negative control for antigen-specific cytotoxicity. Mean and SEM plotted, representative donor, measured in triplicates. N=3 human donors. Tc—Tconv cells, Tr—Treg

FIG. 38A shows the U87 tumor model for CAR-Treg trafficking experimental outline.

FIG. 38B shows hematoxylin and eosin (H&E), CD3, and mCherry staining of U87-CD19 and U87-WT tumors from mice treated with CD19 28ζ CAR-Tregs and IL-2.

FIGS. 38C and 38D show tumor bioluminescent imaging (BLI) of left and right flank tumors of mice. Each line represents either a U87-CD19⁻ or U87-CD19⁺ tumor in an individual mouse. Tr—Treg. FIG. 38C shows mice treated with CD19-28ζ Tregs. FIG. 38D shows mice treated with EGFR-2K Tconv cells.

FIG. 39A shows the skin xenograft model experimental outline.

FIG. 39B shows a vector map of the EGFR 28ζ CAR construct. TM, hinge and transmembrane domain. L, leader sequence.

FIG. 39C shows luciferase-based killing assay using U87 CBG-GFP cells incubated with CAR EGFR 28ζ Tregs and Tconv cells at varying ratios for 16 hours. Representative donor.

FIG. 39D shows images of skin grafts from a representative mouse. Repeated with N=3 donor grafts and N=3 donor T cells, 1 mouse per group.

FIG. 39E shows the size of graft measured as a percentage of the size prior to CAR T cell injection.

FIG. 39F shows H&E histology of sections from grafts 2 weeks after Treg were injected, taken with a 4× objective lens.

FIG. 39G shows H&E and immunohistochemistry (IHC) staining of human CD3 (4×), human CD8 (10×), mCherry (10×) and human Foxp3 (10×), representative images. Tr—Treg

FIG. 39H shows immunohistochemistry of nuclear Foxp3 staining in xenograft of EGFR 28ζ Tregs treated mouse (20× objective lens).

FIG. 40A shows tunnel staining (10×) of skin xenografts from mice after treatment with CAR-Tregs and CAR-Teff cells, representative images. Tr—Treg.

FIG. 40B shows RNAscope of TGF-β1 (10×), IL-10 (10×), PRF1 (10×) and GZMB (10×).

### DETAILED DESCRIPTION

The engineered Treg cells described herein provide surprising advantages over non-engineered Tregs and earlier Treg therapies, displaying an increased half-life and improved efficacy by targeting to the desired sites in a subject, e.g., to affected tissues and/or to immune cells which are overactive and contributing to autoimmune disease. Described herein are T cells, e.g., Treg or immunosuppressive T cells, expressing and/or comprising at least one nucleic acid molecule encoding one or more chimeric antigen receptors (CARs). Further provided herein are specific CAR configurations that are demonstrated to provide Tregs with surprisingly high levels of certain behaviors and/or activities.

Chimeric Antigen Receptors (CARs)

In one aspect of any of the embodiments described herein is an engineered Treg cell comprising a) a first chimeric antigen receptor; and/or b) a nucleic acid encoding said first chimeric antigen receptor.

“Chimeric antigen receptor,” “CAR,” or “CARs” as used herein refers to engineered receptors, which graft an antigen specificity onto cells (e.g., T cells, particularly Treg cells). CARs are also known as artificial T-cell receptors, chimeric T-cell receptors, or chimeric immunoreceptors. In some embodiments of any of the aspects, the CARs of the invention comprise at least one extracellular target-binding domain, a hinge/transmembrane domain, and an intracellular signaling domain, e.g., a costimulation domain. In another embodiment, a CAR can be a bispecific CAR. A bispecific CAR is specific to two different antigens. CARs can comprise multiple different extracellular target-binding domains and/or multiple repeats of the same extracellular target-binding domain.

When expressed in a T cell, CARs have the ability to redirect T-cell specificity and reactivity toward a selected target in a non-MHC-restricted manner, e.g., by exploiting the antigen-binding properties of monoclonal antibodies. The non-MHC-restricted antigen recognition gives T-cells expressing CARs the ability to recognize an antigen independent of antigen processing. In some embodiments of any of the aspects, the extracellular target-binding domain of the CAR is composed of a single chain variable fragment (scFv) derived from fusing the variable heavy and light regions of a monoclonal antibody (e.g., a murine or humanized monoclonal antibody). Alternatively, scFvs may be used that are derived from Fabs (instead of from an antibody, e.g., obtained from Fab libraries). In various embodiments, this scFv is fused to a hinge/transmembrane domain and then to an intracellular signaling domain.

“First-generation” CARs include those that solely provide CD3zeta (CD3ζ) signals upon antigen binding. “Second-generation” CARs include those that provide both costimulation (e.g., CD28 or CD 137) and activation (CD3ζ). “Third-generation” CARs include those that provide multiple costimulatory (e.g., CD28 and CD 137) domains and activation domains (e.g., CD3ζ). In various embodiments, the CAR is selected to have high affinity or avidity for the antigen. A more detailed description of CARs and CAR T cells can be found in Maus et al. Blood 2014 123:2624-35; Reardon et al. Neuro-Oncology 2014 16:1441-1458; Hoyos et al. Haematologica 2012 97:1622; Byrd et al. J Clin Oncol 2014 32:3039-47; Maher et al. Cancer Res 2009 69:4559-4562; and Tamada et al. Clin Cancer Res 2012 18:6436-6445; each of which is incorporated herein by reference in its entirety.

As used herein, the “extracellular target binding domain” refers to a polypeptide sequence found on the outside of the cell sufficient to facilitate binding to a target. The extracellular target binding domain will specifically bind to its binding partner, e.g., the target molecule. Most commonly, the extracellular binding domain of the CAR is composed of a single chain variable fragment (scFv) derived from fusing the variable heavy and light regions of a murine or humanized monoclonal antibody. Alternatively, scFvs may be used that are derived from Fabs (instead of from an antibody, e.g., obtained from Fab libraries). In various embodiments, this scFv is fused to a transmembrane domain, and then to an intracellular signaling domain. As used herein, the terms, “binding domain,” “extracellular domain,” “extracellular binding domain,” “antigen-specific binding domain,” and “extracellular antigen specific binding domain,” are used interchangeably and provide a CAR with the ability to specifically bind to the target antigen of interest, e.g., a target molecule. The binding domain may be derived either from a natural, synthetic, semi-synthetic, or recombinant source.

In some embodiments of any of the aspects, the CARs contemplated herein may comprise linker residues between the various domains, e.g., added for appropriate spacing and conformation of the molecule. In particular embodiments the linker is a variable region linking sequence. A “variable region linking sequence,” is an amino acid sequence that connects the VH and VL domains and provides a spacer function compatible with interaction of the two sub-binding domains so that the resulting polypeptide retains a specific binding affinity to the same target molecule as an antibody that comprises the same light and heavy chain variable regions. CARs contemplated herein, can comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments of any of the aspects, the linker is 1, 2, 3, 4, 5, 6, 7, 8, 9, 10, 11, 12, 13, 14, 15, 16, 17, 18, 19, 20, 21, 22, 23, 24, 25, or more amino acids long.

In some embodiments of any of the aspects, a “domain” refers to a conserved region of the polypeptide sequence that is known to bind a target. In some embodiments of any of the aspects, the domain is not a conserved region of the polypeptide sequence. In some embodiments of any of the aspects, target-binding sequence comprises a ligand of the target or an antibody reagent that specifically binds the target.

As used herein, “ligand” refers to a ligand natively found in the genome. A ligand is a molecule which binds specifically to a portion of a protein and/or receptor. A ligand can be found on the surface of a cell or organelle, or within the cytoplasmic space. Ligand-protein/receptor binding can result in the alteration of the protein and/or receptor, or activate a physiological response, for example, the activation of a signaling pathway. In some embodiments of any of the aspects, the ligand can be non-native to the genome. Optionally, the ligand has a conserved function across at least two species.

The binding domain of the CAR is generally followed by one or more “hinge domains,” which plays a role in positioning the antigen binding domain away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8 (e.g., CD8a), CD4, CD28, and CD7, which may be wild-type hinge regions from these molecules or may be altered. In another embodiment, the hinge domain comprises a CD8a hinge region.

The “transmembrane domain” or “TM domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from (i.e., comprise at least the transmembrane region(s) of) the alpha, beta or zeta chain of the T-cell receptor, CD3ε, CD3ζ, CD4, CD5, CD8α, CD9, CD16, CD22, CD27, CD28, CD33, CD37, CD45, CD64, CD80, CD86, CD 134, CD137, CD152, CD154, and PD-1.

The transmembrane domain of the CAR of the invention described herein is the transmembrane region or fragment thereof of a transmembrane protein (for example Type I transmembrane proteins), an artificial hydrophobic sequence, or a combination thereof. Other transmembrane domains will be apparent to those of skill in the art and may be used in connection with alternate embodiments of the invention. A selected transmembrane region or fragment thereof would preferably not interfere with the intended function of the CAR. As used herein, “fragment thereof” refers to a portion of a transmembrane domain that is sufficient to anchor or attach a protein to a cell surface.

As used herein, a “hinge/transmembrane domain” refers to a domain comprising both a hinge domain and a transmembrane domain. In some embodiments of any of the aspects, the hinge/transmembrane domain or fragment thereof of the CAR described herein comprises a hinge/transmembrane domain selected from the transmembrane domain of CD8 or 4-1BB. In an alternate embodiment of any aspect, the hinge/transmembrane domain or fragment thereof of the CAR described herein comprises a hinge/transmembrane domain selected from the hinge/transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

4-1BB is a membrane receptor protein, also known as CD137, which is a member of the tumor necrosis factor (TNF) receptor superfamily. 4-1BB is expressed on activated T lymphocytes. 4-1BB sequences are known for a number of species, e.g., human 4-1BB, also known as TNFRSF9 (NCBI Gene ID: 3604) and mRNA (NCBI Reference Sequence: NM_001561.5). 4-1BB can refer to human 4-1BB, including naturally occurring variants, molecules, and alleles thereof. In some embodiments of any of the aspects, e.g., in veterinary applications, 4-1BB can refer to the 4-1BB of, e.g., dog, cat, cow, horse, pig, and the like. Homologs and/or orthologs of human 4-1BB are readily identified for such species by one of skill in the art, e.g., using the NCBI ortholog search function or searching available sequence data for a given species for sequence similar to a reference 4-1BB sequence.

In some embodiments of any of the aspects, the hinge/transmembrane domain is a CD8 hinge/transmembrane domain. CD8 is an antigen preferentially found on the cell surface of cytotoxic T lymphocytes. CD8 mediates cell-cell interactions within the immune system, and acts as a T cell co-receptor. CD8 consists of an alpha chain (CD8a or CD8a) and beta chain (CD8b or CD8β). CD8a sequences are known for a number of species, e.g., human CD8a, (NCBI Gene ID: 925) and mRNA (NCBI Ref Seq NM_000002.12). CD8 can refer to human CD8, including naturally occurring variants, molecules, and alleles thereof. In some embodiments of any of the aspects, e.g., in veterinary applications, CD8 can refer to the CD8 of, e.g., dog, cat, cow, horse, pig, and the like. Homologs and/or orthologs of human CD8 are readily identified for such species by one of skill in the art, e.g., using the NCBI ortholog search function or searching available sequence data for a given species for sequence similar to a reference CD8 sequence.

In some embodiments of any aspect, the CARs described herein comprise an intracellular signaling domain. An “intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

As used herein, the term, “co-stimulation domain,” “co-stimulatory signaling domain,” or “co-stimulatory domain,” refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In some embodiments of any of the aspects, a CAR comprises one or more co-stimulatory signaling domains selected from the group consisting of CD28, CD137, and CD134, and a CD3ζ primary signaling domain.

In some embodiments of any of the aspects, the intracellular co-stimulation domain is a 4-1BB intracellular co-stimulation domain. In some embodiments of any of the aspects, the intracellular co-stimulation domain is a CD28 intracellular co-stimulation domain.

In some embodiments of any of the aspects the CAR can alternatively or further comprise a primary signaling domain in the intracellular portion. Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs. Illustrative examples of ITAM containing primary signaling domains that are of particular use in the invention include those derived from TCRζ, FcRγ, FcRβ, CD3γ, CD3δ, CD3ε, CD3ζ, CD22, CD79a, CD79b, and CD66d.

As used herein, “CD3z chain” or “CD3ζ chain” refers to a polypeptide sequence comprising the intracellular domain of CD3z, also known in the art as CD3ζ or CD247. CD3ζ is a component of the T cell receptor CD3 complex, coupling antigen recognition to signal transduction pathways. Sequences for CD3ζ are known in the art for a number of species, e.g., human CD3ζ (NCBI Gene ID: 919) polypeptide (e.g., NCBI Ref Seq NP_000725.1 and NP_932170.1) and mRNA sequences (e.g., NCBI Ref Seq NM_000734.3 and NM_198053.2). In some embodiments of any of the aspects, a CD3ζ can be and/or can comprise a sequence selected from SEQ ID Nos: 1-7.

SEQ ID NO: 1
MKWKALFTAA ILQAQLPITE AQSFGLLDPK LCYLLDGILF
IYGVILTALF LRVKFSRSAD APAYQQGQNQ LYNELNLGRR
EEYDVLDKRR GRDPEMGGKP RRKNPQEGLY NELQKDKMAE
AYSEIGMKGE RRRGKGHDGL YQGLSTATKD TYDALHMQAL
PPR
SEQ ID NO: 2
RVKFSRSAD APAYQQGQNQ LYNELNLGRR EEYDVLDKRR
GRDPEMGGKP RRKNPQEGLY NELQKDKMAE AYSEIGMKGE
RRRGKGHDGL YQGLSTATKD TYDALHMQAL PPR
SEQ ID NO: 3
RVKFSRSAD APAYQQGQNQ LYNELNLGRR EEYDVLDKRR
GRDPEMGGKP RRKNPQEGLY NELQKDKMAE AYSEIGMKGE
RRRGKGHDGL YQGLSTATKD TYDALHM
SEQ ID NO: 4
NQ LYNELNLGRR EEYDVLDKRR GRDPEMGGKP RRKNPQEGLY
NELQKDKMAE AYSEIGMKGE RRRGKGHDGL YQGLSTATKD
TYDALHMQAL PPR
SEQ ID NO: 5
NQ LYNELNLGRR EEYDVLDKRR GRDPEMGGKP RRKNPQEGLY
NELQKDKMAE AYSEIGMKGE RRRGKGHDGL YQGLSTATKD
TYDALHM
SEQ ID NO: 6
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR
SEQ ID NO: 7
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In some embodiments of any of the aspects, a CAR can comprise a) an extracellular target-binding domain; b) a hinge/transmembrane domain; and c) an intracellular co-stimulation domain. In some embodiments of any of the aspects, a CAR can comprise a) an extracellular target-binding domain; b) a hinge/transmembrane domain; c) an intracellular co-stimulation domain, and d) a CD3z chain. In some embodiments of any of the aspects, a CAR can comprise, in order from N-terminus to C-terminus, a) an extracellular target-binding domain; b) a hinge/transmembrane domain; and c) an intracellular co-stimulation domain. In some embodiments of any of the aspects, a CAR can comprise in order from N-terminus to C-terminus, a) an extracellular target-binding domain; b) a hinge/transmembrane domain; c) an intracellular co-stimulation domain, and d) a CD3z chain.

In some embodiments of any of the aspects, wherein it is desired to have high CD39 expression, the engineered Treg cell comprises one or more CARs with a 4-1BB costimulatory domain. In some embodiments of any of the aspects, wherein it is desired to have relatively low CD39 expression, the engineered Treg cell comprises one or more CARs with a) a CD28 costimulatory domain or b) a CD3z chain in the absence of a costimulatory domain.

In some embodiments of any of the aspects, wherein it is desired that LAP expression is lower, and/or does not increase after stimulation of a Treg, the engineered Treg cell comprises one or more CARs with a 4-1BB costimulatory domain. In some embodiments of any of the aspects, wherein it is desired that LAP expression is higher, and/or increases after stimulation of a Treg, the engineered Treg cell comprises one or more CARs with a CD28 costimulatory domain.

In some embodiments of any of the aspects, wherein it is desired to not suppress T cell proliferation and/or cytokine secretion, the engineered Treg cell comprises one or more CARs with a 4-1BB costimulatory domain.

In some embodiments of any of the aspects, wherein it is desired to suppress T cell proliferation and/or cytokine secretion, the engineered Treg cell comprises one or more CARs with a) a CD28 costimulatory domain or b) a CD3ζ chain in the absence of a costimulatory domain. In some embodiments of any of the aspects, wherein it is desired to suppress T cell proliferation and/or cytokine secretion, the engineered Treg cell comprises one or more CARs with a CD3ζ chain in the absence of a costimulatory domain. In other embodiments of any of the aspects, wherein it is desired to suppress T cell proliferation and/or cytokine secretion, the engineered Treg cell comprises one or more CARs with a CD28 costimulatory domain and a CD3ζ chain.

In some embodiments of any of the aspects, wherein immunosuppression in a solid tissue (e.g., liver, skin, or lung) is desired, the engineered Treg cell comprises one or more CARs with a) a CD28 costimulatory domain and b) a CD3ζ chain.

In some embodiments of any of the aspects, the CAR can further comprise an N-terminal leader sequence. As used herein, “leader sequence” refers to a peptide sequence that directs the transport and localization of the protein within a cell, e.g. to a certain cell organelle (such as the endoplasmic reticulum) and/or the cell surface. The leader sequence can be, e.g., a leader sequence and/or a signal peptide of any secreted or transmembrane human protein of type 1 (extracellular N-terminus which allows the transport of a CAR described herein to the cell membrane and cell surface and allows correct localization of the CAR, in particular the extracellular portion on the cell surface; the transmembrane portion inserted into the plasma. membrane and the cytoplasmic portion in the host cell. In some embodiments of any of the aspects, the leader sequence can be cleaved after passage of the endoplasmic reticulum (ER), i.e. is a cleavable leader sequence. In some embodiments of any of the aspects, the leader sequence can be a leader sequence and/or signal peptide from an immunoglobulin chain. In some embodiments of any of the aspects, the leader sequence can be a CD8 leader sequence.

In some embodiments of any of the aspects, the target-binding domain can be a ligand, receptor, antibody reagent, or other molecule that can specifically bind to the target molecule. In some embodiments of any of the aspects, the target-binding domain is an antibody reagent, e.g., a scFv.

Targets and Antigens

First CAR

In some embodiments of any of the aspects, the first CAR comprises an extracellular domain that specifically binds to a first target molecule expressed on the surface of a first target cell. The first target molecule can be a molecule found on the target cell and/or a molecule found exclusively on the target cell. In some embodiments of any of the aspects, the target molecule is a molecule found on higher concentrations or levels on the target cell as compared to other cell types.

In some embodiments of any of the aspects, the first target molecule can be CD19, (e.g. human CD19, NCBI Gene ID: 930). In some embodiments of any of the aspects, the extracellular target-binding domain of the first CAR can be an anti-CD19 antibody reagent, e.g., an anti-CD19 scFv.

First Target Cell

The first target cell can be a cell affected by an autoimmune condition and/or allograft rejection, e.g., if the autoimmune condition is targeting/attacking liver cells, the first target cell can be a liver cell. In another example, the cell can be a skin cell, a blood cell, an endothelial cell, or a lung cell. In some embodiments of any of the aspects, the first target cell can be a cell found in a tissue affected by an autoimmune condition and/or allograft rejection. Such specificity for binding to the first target cell permits the engineered T cells described herein to localize to the site of disease and provide markedly increased efficacy in immunosuppression.

In some embodiments of any of the aspects, the first target molecule can be epidermal growth factor receptor (EGFR).

Second CAR

In some embodiments of any of the aspects, the engineered Treg cell can further comprise a second chimeric antigen receptor; and/or a nucleic acid encoding said second chimeric antigen receptor, wherein the second chimeric antigen receptor comprises an extracellular domain that specifically binds a different target molecule than the first chimeric antigen receptor.

In some embodiments of any of the aspects, the first and second target molecules can be found on the same target cell. Such embodiments can provide greater specificity and activity than a single CAR and reduce off-target effects.

Second Target Cell

In some embodiments of any of the aspects, the second target molecule is expressed on the surface of a second target cell, e.g., a cell which is not the same cell as the first target cell. Such embodiments can provide the ability to target multiple cell types, e.g. to provide even finer localization to the precise location where both cell types are present. In some embodiments of any of the aspects, the second target cell is an immune system cell contributing to and/or causing symptoms or damage associated with an autoimmune condition and/or allograft rejection. Such embodiments provide the ability to localize to both the cells affected by or instigating an immune response as well as the immune cells generating, enacting, and/or permitting an undesired immune response. This double targeting strategy can further improve the efficacy of the engineered T cells.

In some embodiments of any of the aspects, the second target cell, e.g., a target immune system cell, can be a Treg cell. Treg cells can be targeted using extracellular target-binding domains that bind specifically to a target molecule selected from, e.g., CTLA4 (e.g., NCBI Gene ID: 1493); CD25 (e.g., NCBI Gene ID: 3559); CD27 (e.g., NCBI Gene ID: 939); PDL1 (e.g., NCBI Gene ID: 29126); GARP (e.g., NCBI Gene ID: 2615); TGFbeta (e.g., NCBI Gene ID: 7040); and LAP (e.g., NCBI Gene ID: 7040).

In some embodiments of any of the aspects, the second target cell, e.g., a target immune system cell, can be a myeloid derived suppressor cell (MDSC). MDSCs are myeloid-derived cells with immunosuppressive activities that regulate the activity of T cells, dendritic cells, macrophages, and natural killer cells. MDSCs can be targeted using extracellular target-binding domains that bind specifically to a target molecule selected from, e.g., CD32 (e.g., NCBI Gene ID: 2212); CD33 (e.g., NCBI Gene ID: 945), and CD11c (e.g., NCBI Gene ID: 3687).

In other embodiments, the second target cell can be a skin cell, a blood cell, an endothelial cell, or a lung cell.

In some embodiments of any of the aspects, the second target molecule can be epidermal growth factor receptor (EGFR).

Engineered Regulatory T Cells (Tregs)

As used herein, the term “T cell” refers to lymphocytes (white blood cells) that function in cell-mediated immunity. The presence of a T cell receptor (TCR) on the cell surface distinguishes them from other lymphocytes. T cells do not present antigens and rely on other lymphocytes (natural killer cells, B cells, macrophages, dendritic cells) to aid in antigen presentation. Types of T cells include: T helper cells (TH cells), Memory T cells (Tcm, Tern, or Temra), Regulatory T cells (Treg), Cytotoxic T cells (CTLs), Natural killer T cells (NKT cells), gamma delta T cells, and Mucosal associated invariant T cells (MAIT).

Regulatory T cells or Treg cells play an important role for the maintenance of immunological tolerance by suppressing the action of autoreactive effector cells and have been shown to be critically involved in preventing the development of autoimmune reactions. Accordingly, as described herein, a “regulatory T cell” or “Treg” refers to those T cells that have immunoregulatory properties and the ability to suppress the proliferation and/or effector function of other T cell populations. A number of cell surface molecules are used to characterize and define Treg cells as described below herein.

In some embodiments of any of the aspects, a Treg cell can be a T cell expressing one or markers selected from the group consisting of CTLA4; PDL1; LAP; GARP; CD25; and CD27. In some embodiments of any of the aspects, a Treg cell can be a CD8− (e.g., NCBI Gene ID: 925) CD4+ (e.g., NCBI Gene ID: 920) CD25+ (e.g., NCBI Gene ID: 3559) CD127+(e.g., NCBI Gene ID: 3575) cell. In some embodiments of any of the aspects, a Treg cell can be a CD8− CD4dim CD25 hi CD127 low cell. In some embodiments of any of the aspects, a Treg cell can be a CD8− CD4low CD25 hi CD127 low cell.

“X+”, wherein “X” is a cell surface marker, indicates the marker is present in the indicated cell, while “X−” indicates the marker is not present. One skilled in the art will be capable of assessing the molecules present on a cell using standard techniques, for example using immunofluorescence to detect commercially available antibodies bound to the marker molecules. The designators “hi” “high” “lo” “low” or “dim” are used in a similar fashion to indicate a cell with low or high expression of the marker as compared to a larger population of cells. Such designators are often used when sorting or identifying cells by FACS, in which gates can be established to divide cells based on the level of expression of the marker.

In some embodiments of any of the aspects, the engineered cell can be a mammalian cell. In some embodiments of any of the aspects, the engineered cell can be a human cell or a murine cell. In some embodiments of any of the aspects, the engineered Treg cell can be a primary cell. T cells can be obtained from a subject using standard techniques known in the field, for example, T cells are isolated from peripheral blood taken from a patient. In some embodiments of any of the aspects, the engineered Treg cell can be derived from a stem cell, precursor cell, and/or iPSC.

A nucleic acid molecule encoding CAR or other protein described herein can further comprise additional genetic elements that facilitate expression of the protein, e.g., promoters, enhancers, and the like. The nucleic acid molecule can be comprised by a vector, e.g., a viral vector or plasmid and/or integrated into the genome of the cell.

In some embodiments of any of the aspects, the engineered T cell can be autologous to the subject. A T cell, T cell precursor, stem cell, and/or iPSC can be obtained from the subject and engineered as described herein to provide an engineered T cell which is autologous to the subject. The cell can be obtained from the subject immediately before engineering the cell, obtained from a culture of the subject's cells, and/or provided from a sample collected at an earlier date (e.g. a frozen sample).

In some embodiments of any of the aspects, the engineered T cell can be allogenic to the subject. To provide allogeneic cells, the T cell can be further engineered, e.g., engineered to not express an endogenous T cell receptor and/or endogenous MHC complex. Such modifications are known in the art and can be engineered by, e.g., directed mutagenesis, directed deletion/insertions (e.g., via homologous recombination), CRISPR technology or the like. An endogenous T cell receptor and/or endogenous MHC complex gene can be engineered to, e.g., disable the promoter, provide a premature stop codon, or to delete the coding sequence of the gene. In some embodiments of any of the aspects, the cell can be engineered to comprise an inhibitory nucleic acid that inhibits the expression of the endogenous T cell receptor and/or one or more genes of the endogenous MHC complex.

The engineered T cells described herein can be further engineered to promote or increase their immunosuppressive activity. For example, an engineered T cell as described herein can further comprise a) exogenous FoxP3 (e.g. NCBI Gene ID: 50943); CTLA4; PDL1; and/or TGFbeta polypeptides; and/or b) an exogenous nucleic acid encoding FoxP3; CTLA4; PDL1; and/or TGFbeta polypeptides. As described elsewhere herein, a nucleic acid can be provided in a vector and integrated into the genome or maintained episomally.

The engineering of the T cell can be performed in a mature T cell or performed in a T cell precursor cell (e.g., a stem cell or partially differentiated cell) and a T cell then differentiated from the precursor cell. In the case of multiple instances of engineering of the cell, e.g., to provide CARs and to provide exogenous FoxP3, each instance of engineering can be conducted at the same time or at different times. In the case of multiple instances of engineering of the cell, e.g., to provide CARs and to provide exogenous FoxP3, each instance of engineering can be conducted in a cell at the same stage of differentiation or at different stages of differentiation.

A cell, for example a Treg cell, can be engineered to comprise, e.g., any of the CAR polypeptides described herein; or a nucleic acid encoding any of the CAR polypeptides described herein. In some embodiments of any of the aspects, a nucleic acid encoding a CAR polypeptide as described herein is comprised in a lentiviral vector. The lentiviral vector is used to express the CAR polypeptide in a cell using standard infection techniques.

Retroviruses, such as lentiviruses, provide a convenient platform for delivery of nucleic acid sequences encoding a gene, or engineered gene of interest. A selected nucleic acid sequence can be inserted into a vector and packaged in retroviral particles using techniques known in the art. The recombinant virus can then be isolated and delivered to cells, e.g. in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN:978-1-90455-55-4); and Hu and Pathak Pharmacological Reviews 2000 52:493-512; which are incorporated by reference herein in their entirety. Lentiviral system for efficient DNA delivery can be purchased from OriGene; Rockville, Md. In alternative embodiments, the CAR polypeptide of any of the CARs described herein is expressed in a mammalian cell via transfection or electroporation of an expression vector comprising a nucleic acid encoding the CAR. Transfection or electroporation methods are known in the art.

Efficient expression of the CAR polypeptide as described herein can be assessed using standard assays that detect the mRNA, DNA, or gene product of the nucleic acid encoding the CAR. For example, RT-PCR, FACS, northern blotting, western blotting, ELISA, or immunohistochemistry. In some embodiments of any of the aspects, the CAR polypeptide as described herein is constitutively expressed. In some embodiments of any of the aspects, the CAR polypeptide as described herein is inducibly expressed. In some embodiments of any of the aspects, the CAR polypeptide as described herein is encoded by recombinant nucleic acid sequence.

Reduction of Cytotoxicity

According to the invention described herein, the cytotoxicity function of a Treg is reduced, e.g., by reducing or eliminating the expression of a perforin, granzyme, and/or Fas-ligand gene. This can be achieved using any of a number of methods that are known in the art. In one example, perforin sequences (e.g., coding or regulatory sequences, e.g., NCBI Gene ID: 5551), granzyme A (e.g., coding or regulatory sequences, e.g., NCBI Gene ID: 3001) or granzyme B (e.g., coding or regulatory sequences, e.g., NCBI Gene ID: 3002) sequences, and/or Fas-ligand (e.g., coding or regulatory sequences, e.g., NCBI Gene II): 356) are mutated or deleted from the genome of T cells using, for example, gene editing methods. Thus, for example, approaches employing RNA/DNA guided endonucleases (e.g., Clustered. Regularly Interspersed Short Palindromic Repeats (CRISPR)/Cas9, Cpf1, and Argonaute), Transcription Activator-Like Effector (TALE)-nucleases, zinc finger nucleases (ZFN), or meganucleases can be adapted for use in the invention. Further methods of engineering nucleases to achieve a desired sequence specificity, which can be used in the invention, are described, e.g., in Kim (2014); Kim (2012); Belhaj et al. (2013); Urnov et al. (2010); Bogdanove et al. (2011); Jinek et al. (2012) Silva et al. (2011); Ran et al. (2013); Carlson et al. (2012); Guerts et al. (2009); Taksu et al. (2010); and Watanabe et al. (2012); each of which is incorporated herein by reference in its entirety.

In various examples, insertions or deletions are made by gene editing to cause a frame shift mutation, leading to gene knock out (i.e., lack of expression of a functional gene product). In certain examples, such mutations are made to target early coding regions, close to the N-terminus of the protein, in order to maximize disruption and minimize the possibility of low-level protein expression. In various examples, any exon can be targeted for the creation of a frame shift (e.g., an exon coding sequence). As a specific example, a more proximal exon may be targeted.

Alternatively, reduction or elimination of perforin, granzyme, and/or Fas-ligand gene expression can be achieved by the use of an inhibitory nucleic acid. As used herein, an “inhibitory nucleic acid” refers to a nucleic acid molecule that can inhibit the expression of a target gene or mRNA and includes, e.g., double-stranded RNAs (dsRNAs), inhibitory RNAs (iRNAs), and the like. Inhibitory nucleic acid technology is more fully described in, e.g., Wilson, R C, and Doudna, J A (2013) Annual Review of Biophysics 42(217-239) and reference cited therein.

Therapeutic Compositions and Methods

In some embodiments of any of the aspects described herein, a composition comprising Treg cells as described herein comprises no more than 10% CD8+ T cells, e.g., no more than 10%, no more than 5%, no more than 1%, or no detectable CD8+ T cells. In some embodiments of any of the aspects described herein, an engineered Treg cell as described herein is CD8−.

The engineered T cells described herein can be administered to subjects to treat or prevent an autoimmune condition or allograft rejection. In one aspect of any of the embodiments, described herein is a method of treating or preventing an autoimmune condition or allograft rejection in a subject in need thereof, the method comprising administering an engineered Treg cell as described herein to the subject. In one aspect of any of the embodiments, described herein is an engineered Treg cell for administration to a subject in need of treatment or prevention of an autoimmune condition or allograft rejection.

In one aspect of any of the embodiments, described herein is a method of treating or preventing an autoimmune condition or allograft rejection in a subject in need thereof, the method comprising: a) engineering a Treg cell to express at least a first chimeric antigen receptor; b) administering the engineered Treg cell to the subject. In one aspect of any of the embodiments, described herein is a method of treating or preventing an autoimmune condition or allograft rejection in a subject in need thereof, the method comprising: a) engineering a Treg cell to express at least a first chimeric antigen receptor; b) stimulating the Treg cell resulting from step a); and c) administering the stimulated Treg cell to the subject. In some embodiments of any of the aspects, the T cell can be further engineered to comprise a second CAR as described herein, and/or a further modification as described herein.

In any of the examples and embodiments described herein, the engineered Tregs including a CAR with a co-stimulatory domain in addition to the signaling domain (e.g., a CAR including CD28 co-stimulatory domain and a CD3ζ signaling domain) are capable of mediating a zone of immunosuppression in a tissue (e.g., liver, skin, or lung). In particular, the engineered CAR-Tregs traffic to the site of antigen expression and mediate immunosuppression in this zone. The immunosuppressive activity of the engineered CAR-Tregs is dominant in a tissue, even when the tissue is targeted by large numbers of cytotoxic T cells that would quickly destroy it.

Methods of stimulating T cells, e.g., stimulating T cells ex vivo are known in the art. In some embodiments of any of the aspects, stimulating the T cell can comprise contacting the cell with CD3 (e.g., a complex of NCBI Gene IDs: 915, 916, and 915) and/or CD28 (e.g., NCBI Gene ID: 940) polypeptides. The polypeptide(s) can be displayed, e.g., on a bead, cell, or other surface.

As used herein, “autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include cancer cells. An autoimmune condition, disease, or disorder is caused by the inability of one's immune system to distinguish between a foreign cell and a healthy cell. This results in one's immune system targeting one's healthy cells for programmed cell death. Non-limiting examples of an autoimmune disease or disorder include inflammatory arthritis, type 1 diabetes mellitus, multiples sclerosis, psoriasis, inflammatory bowel diseases, SLE, and vasculitis, allergic inflammation, such as allergic asthma, atopic dermatitis, and contact hypersensitivity. Other examples of auto-immune-related disease or disorder, but should not be construed to be limited to, include rheumatoid arthritis, multiple sclerosis (MS), systemic lupus erythematosus, Graves' disease (overactive thyroid), Hashimoto's thyroiditis (underactive thyroid), celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis and fibromyalgia (FM). In some embodiments of any of the aspects, the autoimmune condition can be diabetes, neurologic disease, or graft vs host disease.

In some embodiments of any of the aspects, the subject has or has been diagnosed with graft vs host disease (GVHD). In a further such embodiment, the subject being treated with the methods described herein is an organ or tissue transplant recipient. In some embodiments of any of the aspects, the methods described herein are used for increasing transplantation tolerance in a subject. In some such embodiments, the subject is a recipient of an allogenic transplant. The transplant can be any organ or tissue transplant, including but not limited to heart, kidney, liver, skin, pancreas, bone marrow, skin or cartilage. “Transplantation tolerance,” as used herein, refers to a lack of rejection of the donor organ by the recipient's immune system.

Administration

The compositions and methods described herein can be administered to a subject having or diagnosed as having an autoimmune disease or disorder and/or at risk of allograft rejection. In some embodiments of any of the aspects, the methods described herein comprise administering an effective amount of engineered Treg cells as described herein to a subject in order to alleviate a symptom of an autoimmune disease or disorder. As used herein, “alleviating a symptom” of a condition is ameliorating any condition or symptom associated with the condition. As compared with an equivalent untreated control, such reduction is by at least 5%, 10%, 20%, 30%, 40%, 50%, 60%, 70%, 80%, 90%, 95%, 99% or more as measured by any standard technique. A variety of means for administering the compositions described herein to subjects are known to those of skill in the art.

In some embodiments of any of the aspects, the methods described herein reduce the level and/or activity of CD8+ T cells in the subject. In some embodiments of any of the aspects, the methods described herein reduce cytokine production in the subject. In some embodiments of any of the aspects, the methods described herein reduce T cell proliferation in the subject.

The term “effective amount” as used herein refers to the amount of engineered Treg cells as described herein needed to alleviate at least one or more symptom of the disease or disorder, and relates to a sufficient amount of pharmacological composition to provide the desired effect. The term “therapeutically effective amount” therefore refers to an amount of engineered Treg cells as described herein that is sufficient to provide a particular anti-disease effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount.” However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of engineered Treg cells as described herein, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for bone marrow testing, among others. The dosage can be determined by a physician and adjusted, as necessary, to suit observed effects of the treatment.

“Unit dosage form” as the term is used herein refers to a dosage for suitable one administration. By way of example a unit dosage form can be an amount of therapeutic disposed in a delivery device, e.g., a syringe or intravenous drip bag. In some embodiments of any of the aspects, a unit dosage form is administered in a single administration. In some embodiments of any of the aspects, more than one unit dosage form, e.g., two injections, can be administered simultaneously.

In some embodiments of any of the aspects, engineered Treg cells as described herein are administered as a monotherapy, e.g., another treatment for the autoimmune condition or allograft rejection is not administered to the subject.

It can generally be stated the engineered Treg cells as described herein may be administered at a dosage of 10⁴ to 10⁹ cells/kg body weight, in some instances 10⁵ to 10⁶ cells/kg body weight, including all integer values within those ranges. Engineered Treg cells as described herein may also be administered multiple times at these dosages. The cells can be administered by using infusion techniques that are commonly known in immunotherapy (see, e.g., Rosenberg et al., New Engl. J. of Med. 319:1676, 1988).

In certain aspects, it may be desired to administer engineered Treg cells as described herein to a subject and then subsequently redraw blood (or have an apheresis performed), activate or stimulate Treg cells therefrom according to the present invention, and reinfuse the patient with these activated and expanded Treg cells. This process can be carried out multiple times every few weeks. In certain aspects, Treg cells can be activated from blood draws of from 10 cc to 400 cc. In certain aspects, Treg cells are activated from blood draws of 20 cc, 30 cc, 40 cc, 50 cc, 60 cc, 70 cc, 80 cc, 90 cc, or 100 cc.

The administration of the subject may be carried out in any convenient manner, including by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In one aspect, the engineered Treg cell compositions of the present invention are administered to a patient by intradermal or subcutaneous injection. In some embodiments of any of the aspects, the engineered Treg cells as described herein are administered by i.v. injection. The engineered Treg cells as described herein may be injected directly into a tumor, lymph node, or site of autoimmune reaction/disease.

In some embodiments of any of the aspects, subjects may undergo leukapheresis, wherein leukocytes are collected, enriched, or depleted ex vivo to select and/or isolate the cells of interest, e.g., T cells. These T cell isolates may be expanded by methods known in the art and engineered as described herein, thereby creating the engineered Treg cells of the invention.

In some embodiments of any of the aspects, lymphodepletion is performed on a subject, e.g., prior to administering one or more engineered Treg cells as described herein. In embodiments, the lymphodepletion comprises administering one or more of melphalan, Cytoxan, cyclophosphamide, and fludarabine.

The dosage of the above treatments to be administered to a patient will vary with the precise nature of the condition being treated and the recipient of the treatment. The scaling of dosages for human administration can be performed according to art-accepted practices.

In some embodiments of any of the aspects, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80%, or at least 90% or more. In some embodiments of any of the aspects, no additional treatments are administered following the initial treatment.

The dosage of the engineered Treg cells as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to any of the engineered Treg cells as described herein. The desired dose or amount of activation can be administered at one time or divided into subdoses, e.g., 2-4 subdoses and administered over a period of time, e.g., at appropriate intervals through the day or other appropriate schedule. In some embodiments of any of the aspects, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. Engineered Treg cells as described herein can be administered over a period of time, such as over a 5 minute, 30 minute, 90 minute, 180 minute, or 240 minute, or more period.

The dosage ranges for the administration of engineered Treg cells as described herein according to the methods described herein depend upon, its potency, and the extent to which symptoms, markers, or indicators of a condition described herein are desired to be reduced. The dosage should not be so large as to cause adverse side effects, such as pathological immunosuppression. Generally, the dosage will vary with the age, condition, and sex of the patient and can be determined by one of skill in the art. The dosage can also be adjusted by the individual physician in the event of any complication.

Combination Therapies

The engineered Treg cells as described herein may be used in combination with other known agents and therapies. Administered “in combination,” as used herein, means that two or more different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments of any of the aspects, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments of any of the aspects, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The engineered Treg cells as described herein and the at least one additional therapeutic agent can be administered simultaneously, in the same or in separate compositions, or sequentially. For sequential administration, engineered Treg cells as described herein can be administered first, and the additional agent can be administered second, or the order of administration can be reversed. The engineered Treg cells as described herein and/or other therapeutic agents, procedures or modalities can be administered during periods of active disorder, or during a period of remission or less active disease. The engineered Treg cells as described herein can be administered before another treatment, concurrently with the treatment, post-treatment, or during remission of the disorder.

When administered in combination, the engineered Treg cells as described herein and the additional agent (e.g., second or third agent), or all, can be administered in an amount or dose that is higher, lower or the same than the amount or dosage of each agent used individually, e.g., as a monotherapy. In certain embodiments, the administered amount or dosage of the engineered Treg cells as described herein, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of engineered Treg cells as described herein, the additional agent (e.g., second or third agent), or all, that results in a desired effect is lower (e.g., at least 20%, at least 30%, at least 40%, or at least 50% lower) than the amount or dosage of each agent used individually, e.g., as a monotherapy, required to achieve the same therapeutic effect. In further aspects, engineered Treg cells as described herein described herein may be used in a treatment regimen in combination with immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation, peptide vaccine, such as that described in Izumoto et al., 2008, J. Neurosurg. 108:963-971.

Efficacy

The efficacy of administering engineered Treg cells as described herein in, e.g., the treatment of a condition described herein, or to induce a response as described herein (e.g., a reduction in autoimmunity) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent (e.g., engineered Treg cells as described herein) can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of an autoimmune condition. When using an experimental animal model, efficacy of treatment is evidenced when a statistically significant change in a marker is observed.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The terms “decrease,” “reduced,” “reduction of,” or “inhibit,” are all used herein to mean a decrease by a statistically significant amount. In some embodiments of any of the aspects, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “enhance,” or “activate,” are all used herein to mean an increase by a statically significant amount. In some embodiments of any of the aspects, the terms “increased,” “increase,” “enhance,” or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments of any of the aspects, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of autoimmune conditions. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for a condition or one or more complications related to the condition or a subject who does not exhibit risk factors.

A “subject in need” of treatment for a particular condition can be a subject having that condition, diagnosed as having that condition, or at risk of developing that condition.

As used herein, the term “chimeric” refers to the product of the fusion of portions of at least two or more different polynucleotide molecules. In some embodiments of any of the aspects, the term “chimeric” refers to a gene expression element produced through the manipulation of known elements or other polynucleotide molecules

In some embodiments of any of the aspects, “activation” or “stimulation” can refer to the state of a T cell that has been sufficiently stimulated to induce detectable cellular proliferation. In some embodiments activation can refer to induced cytokine production. In other embodiments, activation can refer to detectable effector functions. The term “activated T cells” refers to, among other things, T cells that are undergoing cell division. T cells can be stimulated by contacting them with a stimulatory ligand.

A “stimulatory ligand,” as used herein, refers to a ligand that can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. the activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Be; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments of any of the aspects, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments of any of the aspects, the polypeptide described herein can be a variant of a sequence described herein. In some embodiments of any of the aspects, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, inhibitors of the expression of a given gene can be an inhibitory nucleic acid. As used herein, “inhibitory nucleic acid” refers to a nucleic acid molecule which can inhibit the expression of a target, e.g., double-stranded RNAs (dsRNAs), inhibitory RNAs (iRNAs), and the like.

Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA (or modified nucleic acids as described below herein) and which mediates the targeted cleavage of an RNA transcript via an RNA-induced silencing complex (RISC) pathway. In some embodiments of any of the aspects, an iRNA as described herein effects inhibition of the expression and/or activity of a target. In some embodiments of any of the aspects, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA. In some embodiments of any of the aspects, administering an inhibitor (e.g. an iRNA) to a subject results in a decrease in the target mRNA level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

Exemplary embodiments of types of inhibitory nucleic acids can include, e.g., siRNA, shRNA, miRNA, and/or amiRNA, which are well known in the art.

In some embodiments of any of the aspects, the RNA of an iRNA, e.g., a dsRNA, is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments of any of the aspects, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-].

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

Modified RNAs can also contain one or more substituted sugar moieties. The iRNAs, e.g., dsRNAs, described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO] mCH3, O(CH2).nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments of any of the aspects, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar.

An inhibitory nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

The preparation of the modified nucleic acids, backbones, and nucleobases described above are well known in the art.

Another modification of an inhibitory nucleic acid featured in the invention involves chemically linking to the inhibitory nucleic acid to one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

Where naturally occurring polypeptides and nucleic acids (or fragments thereof) are described herein, it is contemplated herein that naturally occurring homologs, orthologs, and alleles of the reference polypeptide and/or nucleic acid can be used in alternative embodiments. Sequences of such homologs, orthologs, and alleles are readily obtained by sequence homology searches or querying databases such as that maintained by NCBI.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, a nucleic acid encoding a polypeptide as described herein (e.g. a CAR polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons). The elements found in a vector, e.g., an expression vector, can be operably linked.

As used herein, the term “operably linked” refers to a first polynucleotide molecule, such as a promoter, connected with a second transcribable polynucleotide molecule, such as a gene of interest, where the polynucleotide molecules are so arranged that the first polynucleotide molecule affects the function of the second polynucleotide molecule. The two polynucleotide molecules may or may not be part of a single contiguous polynucleotide molecule and may or may not be adjacent. For example, a promoter is operably linked to a gene of interest if the promoter regulates or mediates transcription of the gene of interest in a cell.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments of any of the aspects, be combined with other suitable compositions and therapies. In some embodiments of any of the aspects, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. an autoimmune condition. The term “treating” includes reducing or alleviating at least one adverse effect or symptom of a condition, disease or disorder associated with, e.g., an autoimmune condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments of any of the aspects, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-O-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, A D A M Kruisbeek, David H Margulies, Ethan M Shevach, Warren Strobe, (eds.) John Wiley and Sons, Inc., 2003 (ISBN 0471142735, 9780471142737), the contents of which are all incorporated herein by reference in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

The following are examples of the methods and compositions of the invention. It is understood that various other embodiments may be practiced, given the general description provided above.

### Example 1. Experimental Design

Regulatory T cells (Tregs) can suppress the cytotoxicity and proliferation of conventional T cells and inflammatory responses, both through contact-dependent mechanisms and contact-independent methods such as secretion of immunosuppressive cytokines (such as IL-10, TGFβ) and acting as sinks for proliferative cytokines (such as IL-2). Tregs may impair anti-tumor immunity, but lack of Tregs can result in autoimmunity, including organ- or site-specific immunity, or accelerate allograft organ or HSCT rejection. In patients with autoimmunity (i.e. diabetes, neurologic disease, or graft-vs-host disease) or allograft rejection, Tregs have therapeutic potential and are thought to ameliorate disease. Infusions of autologous and allogeneic Tregs have been performed in the clinical setting, but these have a short half-life and do not necessarily traffic to the sites desired. Described herein is a platform to genetically modify human Treg cells that (1) increases their half-life by engineering costimulatory and transcriptional factors, and (2) directs them to specific sites based on expression of a surface targeting moiety (such as a chimeric antigen receptor).

It is contemplated herein that:
a. the selection of intracellular signaling domains affects the stability of the Treg function and phenotype
b. the selection of specific targeting moieties in the form of chimeric antigen receptors enables both ex vivo specific activation of Tregs and in vivo activation at desired sites
c. the fate of the Treg can be manipulated by inclusion or exclusion of additional transcription factors (i.e. FoxP3)
d. Tregs can be further genetically modified or edited to generate allogeneic “off-the-shelf” Tregs
e. Tregs can be further genetically modified to exclude specific T cell functions (such as cytotoxicity, by removing the perforin/granzyme/Fas-Ligand genes).

The method and compositions described herein are contemplated for:
a. use as therapeutic products for site-specific organ-targeted autoimmunity
b. use as therapeutic products for hematologic autoimmunity (i.e. graft vs host disease in allogeneic HSCT recipients)
c. use as prophylactic product to prevent allograft rejection (solid organ transplants), graft-vs-host disease, or autoimmune disease in high-risk individuals (such as Type I diabetes mellitus, multiple sclerosis, rheumatoid arthritis, celiac disease, or inflammatory bowel disease including Crohn's disease and ulcerative colitis).

### Example 2. Sorting, Expanding, and Transducing T Cell Populations

CD4⁺ T cells were negatively selected from healthy donor leukopaks using RosetteSep Kits with a Ficoll gradient (Stemcell Technologies®) and enriched for CD25+ cells using CD25-PE antibody staining followed by anti-PE microbead selection according to the manufacturer provided protocol (Miltenyi). Not CD25-enriched, CD25-enriched, and CD25-depleted T cells were stained separately at 4×10⁶ cells/100 μl in PBS with 2% heat-inactivated fetal bovine serum (FBS, Gibco® Life Technologies) for 30 min at 4° C. with 50 μl/100 μl brilliant violet staining buffer (BioLegend®), 5 μl/100 μl CD127-BV711, 5 μl/100 μl CD4-BV510 and 2.5 μl/100 μl CD8-APC-H7 (clones OKT3-biolegend, OKT4-biolegend and SK1-BD Pharmingen). Stained cells were washed and re-suspended in HBSS supplemented with 25 mM HEPES and 1% FBS with 4′,6-diamidino-2-phenylindole (DAPI) prior to sorting. Tregs were purified from CD25 enriched cells by fluorescence-activated cell sorting (FACS) for live, CD4⁺ CD8⁻ CD25⁺⁺ CD127^{low} and T conventional were sorted from CD25-depleted by live, CD4⁺ CD8⁻ CD25^{low} into 50% FBS in PBS. Non-enriched cells were used to draw the CD25⁺⁺ sorting gates, which were defined as the level of CD25 expression where a shift to slightly lower for CD4 staining could be seen. 2×10⁵ sorted Tregs and Tconv cells were used to stain surface levels of CD39, LAP, LAG3 and CTLA4 and intracellular Foxp3 analyzed by flow cytometry.

Tregs and Tconv cells were then expanded with anti-CD3/anti-CD28 beads (Dynabead Human Treg expander or Dynabead Human T-activator beads respectively, Gibco®) in CTS™ OpTmizer™ T Cell Expansion Serum Free Media (Thermo Fisher Scientific®) supplemented with 2% human serum (Access Cell Culture LLC), 1× GlutaMAX™ (Thermo Fisher Scientific®), Penicillin-Streptomycin 100 U/ml (Thermo Fisher Scientific®) and recombinant human IL-2 (Peprotech®, 300 IU/ml and 20 IU/ml respectively). One day post-sort, T cells were transduced at an MOI of 5 with lentivirus carrying one of the 5 CAR constructs with a humanized scFv that binds human CD19 and intracellular domains: 4-1BB CD28ζ, and Δζ. The CD28ζ-Foxp3 construct expressed transcription factor Foxp3, which was inserted behind a T2A sequence in the plasmid before second T2A element followed by mCherry.

T cells were expanded for one week with beads and then de-beaded and rested for another week. Media was added every 2-3 days to maintain cells at a concentration of 1×10⁶-2×10⁶ T cells/ml. IL-2 was replaced every 2-3 days. Assays were performed on never-frozen Tregs on day 14-15 in supplemented OpTmizer™ with no IL-2 or in RPMI-1640 with 1× GlutaMAX and 25 mM HEPES (Gibco®, Life Technologies), supplemented with 10% EBS and 100 U/ml penicillin-streptomycin (R10) as stated. For all assays, percent transduction was measured by determining the percentage of mCherry⁺ T cells by flow cytometry analyzed on a BD Fortessa x-20. All CAR T cells were normalized to the same % CAR positive by adding appropriate numbers of expanded UT Treg or Tconv cells. All experiments were performed with CAR transduction above 50%.

### Example 3. Cell Lines

Human embryonic kidney 293 (HEK293T), K562, U87 and Nalm6 cell lines were purchased from American Tissue Culture Collection (ATCC), HEK293T, K562 and Nalm6 cells were expanded in R10. U87 cells were grown in Eagles minimum essential media (ATCC) supplemented with 10% FBS. Cell lines were lentivirally transduced to express the click beetle green luciferase and green fluorescent protein (GFP) under control of the EF-1α promoter followed by single cell sorting to grow a clonal population. Lentiviral particles, produced to express EGFRvIII, human CD19 or membrane-bound OKT3 scFv under the control of the EF-1α, were used to generate U87-CD19, U87-EGFRvIII K562-CD19 and K562-OKT3 by transduction and single cell sorting for positive populations by FACs. U87 and HEK293T cells were passaged using 0.25% and 0.05% Trypsin-EDTA (Thermo Fisher Scientific®) respectively. Target cells were irradiated with 10,000 rads and frozen in FBS with 10% DMSO to be thawed prior to stimulation of CAR T cells. Cell lines were tested for mycoplasma contamination every 3 months.

### Example 4. Construct Generation and Lentivirus Production

CD19, EGFRvIII and EGFR-specific CARs were synthesized and cloned into a third-generation lentiviral plasmid backbone under the regulation of a human EF-1α promoter (GenScript® USA Inc). Replication-defective lentiviral vectors were produced by four plasmids co-transfected into HEK293T cells using TransIT-2020 transfection reagent (Mirus). Supernatants were collected 24 h and 48 h after transfection. Virus was filtered and then concentrated by ultracentrifugation. Vector was harvested and stored at −80° C.

### Example 5. Flow Cytometry Reagents and Analysis

Fluorescent anti-CD3 (OKT3), anti-CD4 (OKT4), anti-CD8 (SK1), anti-CD69 (FN50), anti-LAP (TW4-2F8), anti-CD137 (4B4-1) and anti-CTLA4 (L3D10) antibodies were purchased from Biolegend®, while anti-CD25 (2A3), anti-CD127 (HIL-7R-M21) and anti-CD107a (H4A3) fluorescent antibodies were purchased from BD Biosciences®. Cells were surface stained in 2% FBS PBS for 30 min at 4° C. and DAPI or 7AAD was added prior to running samples if no other live dead stain was used. For Foxp3 intracellular staining, anti-Foxp3 (PCH101) antibodies were purchased from eBioscience®. Blue, Aqua or Violet LIVE/DEAD fixable dyes (Thermo Fisher Scientific®) were used to stain dead cells in PBS for 20 min at 4° C. before surface staining and fixation. After surface staining, T cells were fixed according to eBioscience® Foxp3 transcription factor staining kit's recommended protocol. Briefly, cells were fixed and permeabilized for 45 min and then washed in permeabilization buffer and blocked with rat serum 2 μl/100 μl for 15 min and room temperature (RT). Fixed cells were stained for 30 min with 2 μl of Foxp3 antibody/100 μl at 4° C. Fluorescence was measured on a BD Fortessa x-20 and data were analyzed using FlowJo® (Tree Star).

### Example 6. TCR Vs. CAR Restimulation

Assays were performed in supplemented OpTmizer™ media for all experiments unless otherwise mentioned. For methylation, phenotypic, and proliferation analysis of long-term activated CAR T cells, 1×10⁶ CAR Treg or Tconv cells were stimulated with irradiated K562 cells expressing anti-CD3 (OKT3) or CD19 at a 1:1 T cell-to-target ratio in a 12 well plate. Cells were maintained in culture at a concentration of 5×10⁵-2×10⁶ cells/ml. T cells were counted using a LUNA-FL dual florescence cell counter (Logos Biosystems) and analyzed by flow cytometry to account for live K562 cells 3 times a week for 2 weeks to document long term expansion. For phenotypic analysis, surface and intracellular staining for markers CD39, CTLA4, LAGS, LAP and Foxp3 were measured by flow cytometry pre-(day 14) and 9 days' post (day 23) K562 stimulation. For methylation analysis, T cells were also sorted by mCherry⁺ and CD3⁺ and then frozen at −80° C. for DNA methylation analysis.

For proliferation assays measured by violet dilution, cells were washed in PBS and labeled with 1 ml of cell trace violet dye at a concentration of 1 μM in PBS for 10 min at room temperature on day 14. Labeling was stopped by incubating cells for 1 minute with 1 ml of FBS and for a further 10 minutes, after the addition of 10 ml of R10. Tregs were then washed three times before resuspending in OpTmizer™ media supplemented 300 IU/ml of IL-2 at 1×10⁶ CAR T cells/ml. 100,000 cells were plated per well with 100,000 irradiated Nalm6-target cells and incubated at 37° C. for 3 days. 100 μl of OpTmizer™ was added on day 2. T cells were washed before staining with CD3-APC for 30 min at 4° C. DAPI was added prior to performing flow cytometry with a high through-put plate reader on a Fortessa x-20. The percentage of violet low cells proliferating was calculated and represented as a ratio normalized to the number of violet low cells in the non-stimulated condition.

For short-term activation assays (24-hour), Tregs were grown in reduced IL-2 media (20 units/ml) from day 12 to 14. On day 14, 1×10⁵ T cells/well were activated in a 96-well round-bottom plate with either no target cells or at a 2:1 T cell-to-target ratio with irradiated K562-CD19 or K562-OKT3 cells in a final volume of 200 μl/well with technical triplicates. Cells were incubated at 37° C. for 24 hours. Triplicate wells were pooled and stained for CD3, CD4, CD69, LAP and 4-1BB (CD137) to be measured by flow cytometry.

### Example 7. DNA Methylation Analysis

5×10⁵ Tregs and Tconv cells were sorted by CD3 and mCherry expression, except in the case of UT T cells, which were sorted on CD3 only. Sorted cells were then washed in PBS and snap-frozen before shipping to EpigenDx for methylation analysis. The methylation status of CpG motifs across the Foxp3 TSDR, CTLA4 and IKZF2 loci was assessed by targeted next-generation bisulfite sequencing using the EpigenDx Human Foxp3 methylation panel. % methylation at each CpG site was averaged and then represented as an average from 2-3 human donors/group, only female donors were used for TSDR methylation analysis.

### Example 8. Cytokine Detection by Luminex®

For cytokine release assays, Tregs or Tconv cells were stimulated in a 96-well round-bottom plate with 100,000 CAR T-cells/well combined with irradiated K562-OKT3 or K562-CD19 target cells at a CAR T cell-to-target ratio of 2:1 in a total volume of 200 μl. Supernatants were harvested after 24 hours and frozen at −80° C. 50 μl of supernatant/sample was analyzed for cytokine levels using FLEXMAP 3D® platform from Lumina Instrumentation (Thermo Fisher Scientific®) according to manufacturer's instructions with a panel of the following cytokines: IL-1β, IL-2, IL-4, IL-5, IL-6, IL12p70, IL-13, IL-18, IFN-γ, GM-CSF, TNF-α and IL-10. Plates were read using xPONENT Software 4.1. All samples were in measured in technical triplicates and with N≥3 normal donors. Technical triplicates were averaged before graphing with Prism® (GraphPad® software).

### Example 9. Suppression Assays

Tregs were violet-labeled as described for TCR vs CAR re-stimulation and used in mixed lymphocyte reactions (MLRs) with CFSE-labeled Teff cells (following the same protocol for violet labeling, CFSE cell trace, Invitrogen®, 1 μM staining concentration). Tregs were titrated in a 96-well plate with 5×10⁴ Teff cells/well and 1×10⁵ irradiated target cells/well in R10. Cells were left in an incubator at 37° C. for 3 days unless otherwise mentioned with 100 μl of media added on day 2. In the case where beads were used for the proliferation assay, anti-CD3/anti-CD28 beads (Dynabead: Human T-activator beads) were used at a 1:10 bead-to-Teff cell ratio. To analyze MLR suppression assays, cells were stained with CD3-APC for 30 min at 4° C. and DAPI was added prior to flow cytometry on a Fortessa x-20 with a high throughput plate reader. The percentage of proliferating cells in any condition (x) was calculated as the % of CFSE low cells of the total mCherry⁺ CFSE (Violet⁻) Teff cells in condition(x) of the number of CFSE low cells of mCherry⁺ CFSE Teff cells in the no Treg condition. Experiments were run in technical triplicates with N≥3 normal human donor T cells. Inhibition of cytokine secretion was measured from the supernatant of the MLRs described above in technical triplicates. Cytokines were measured from 50 μl of supernatant using the Luminex® assay described above. For IL-10 blocking assays, IL-10 antibody (LEAF purified, clone JES3-19F1, Biolegend®) was added to wells at a final concentration of 10 μg/ml.

### Example 10. IL-2 Sink Assay

Tregs or Tconv cells were plated at 1×10⁵ CAR T cells/well with technical triplicates in a 96-well round-bottomed plate and stimulated with 1×10⁵ irradiated Nalm6 or no stimulation. Also included was a media only condition where no T cells were added. All cells were in OpTmizer™ with a final starting concentration of 50 IU/ml IL-2 in 200 μl. Cells were incubated for 40 hours at 37° C. after which, supernatants were frozen at −80° C. Cytokine levels were measured from 50 μl of supernatant via Luminex® (described in Example 8).

### Example 11. Cytotoxicity Assays

Luciferase based killing assays were performed by titrating each CAR construct, Tconv or Tregs in a 96-well plate and then adding 2×10⁴ CBG-GFP expressing target cells/well (Nalm6, U87, U87-CD19 or U87-EGFRvIII). Cells were lysed after 15 hours in culture and the live target cells were quantified by BLI after the addition of D-Luciferin. Percent specific lysis was calculated for any condition (x) as

$$\frac{BLI ( target cells alone ) - BLI ( condition x )}{BLI ( target cells alone )} .$$

For the inhibition of granzyme/perforin pathways, we used granzyme-perforin axis inhibitor, concanamycin A (CMA, 200 nM; Sigma) and Granzyme B-specific Inhibitor I (Z-AAD-CMK, 50 μM; Calbiochem). Inhibitor concentrations were chosen based on previously published studies investigating cytotoxicity by granzymes (Choi et al., Cancer Immunol Res. 2013; 1(3):163). Prior to use, each inhibitor was found to have insignificant effects on the viability of Tregs following an 18 h incubation at 37° C. as assessed by LUNA-FL dual florescent counter. In inhibitor assays, all samples including the target-alone samples were incubated with the inhibitor to account for the effects of the inhibitor on tumor cell viability. Assays were run in technical triplicates.

### Example 12. Degranulation Assays

Degranulation of Tregs and Tconv cells was calculated by incubating 3×10⁵ T cells in a 24-well plate with live Nalm6 at a 1:1 Target-to-T cell ratio for 6 hours at 37° C. in media with CD107a (7 μl/well) and befelden A (BD golgiplug 1 μl/ml). PMA inomycin (cell stimulation cocktail, eBioscience®) was used at 1× concentration in culture for 2 hours. Cells were stained with live/dead fixable stain according to manufacturer's instructions (Invitrogen®) followed by surface staining for CD3 and CD4. To determine the Foxp3 expression levels of degranulating cells, we fixed and permeabilized cells and stained for Foxp3 as described in the flow cytometry methods. Both unfixed, surface only stain as well as fixed and permeabilized cells were analyzed by flow cytometry on a Fortessa x-20.

### Example 13. Digital Droplet PCR

CD19 CAR T cells were transduced, expanded and rested for 7 days. 1×10⁶ cells were stimulated with 1×10⁶ Nalm6 cells over 24 hours and then stained with CD4 antibody. 5×10⁵ cells were collected by FACs and resuspended in 350 μl of lysis buffer with 1% 2-mercaptoethanol. RNA was extracted and purified using Aurum Total RNA Mini Kit Bio-Rad®) and cDNA was generated using iScript Reverse Transcription Supermix (Bio-Rad®). Digital Droplet PCR was performed using ddPCR supermix with no dUTPs (Bio-Rad®) with a QX200 Droplet Digital PCR (ddPCR™) System (Bio-Rad®) platform for quantification. Droplet generation, PCR and detection of positive droplets were performed according to manufacturer's instructions (Instruction Manual, QX200™ Droplet Generator-Bio-Rad®).

The PCR cycling protocol was according the manufacturer's instructions with a 57° C. melting temperature. Human TBP was used at as the reference gene in each reaction, (HEX fluorophore: TBP PrimePCR™ ddPCR™ Expression Probe Assay: Unique Assay ID: dHsaCPE5058363 (Bio-Rad®)). The following FAM fluorophore primer probes were used:

GZMB: PrimeTime Std® qPCR Assay unique assay ID Hs.PT.58.26439821.g (IDT)

GZMA: PrimePCR™ PCR Primers unique assay ID dHsaCPE5047756 (BIO-RAD®)

PFR1: PrimePCR™ PCR Primers unique assay ID dHsaCPE5030232 (BIO-RAD®)

### Example 14. Immunohistochemistry

Tissue was collected and fixed in 10% formalin for 24 hours followed by standard paraffin embedding. 5 μm paraffin-embedded tissue sections on glass slides were baked at 60° C. for 30 minutes, followed by deparaffinization in xylene and rehydration in graded alcohol into water. After washing with TBS/Tween 20, antigen retrieval was performed by boiling the slides in 10 mM Sodium Citrate buffer pH=6.0 for 30 minutes. Endogenous peroxidase activity was quenched with Dual Endogenous Enzyme Block (DAKO) for 5 minutes. After washing, tissue sections were incubated with 1:100 dilution of mCherry rabbit polyclonal antibody (Abcam®, ab183628) or 1:400 dilution of CD3 rabbit polyclonal antibody (Dako A0452) in 1% TBS/BSA inside a humidified chamber 1 hour at room temperature. After washing, slides were incubated with HRP Labelled anti Rabbit Polymer (Dako) 30 min at RT. After washing the DAB+ reagent (DAKO) was added with monitoring for 5-10 minutes. After washing, counterstain was done using Harris type Hematoxylin. Slides were briefly dehydrated and then mounted with Histomount solution (Life Technology, 008030).

### Example 15. Automation RNA In Situ Hybridization (ISH) Assay

Automated RNA-ISH assay was performed using the RNAscope 2.5 LS Reagent Kit-Brown from Advanced Cell Diagnostics (ACD) (Catalogue No. 322100) on the BondRx platform. 5-μm sections of FFPE tissue were mounted on Surgipath X-tra glass slides, baked for 1 hour at 60° C., and placed on the BOND RX for processing. On the BOND RX, the staining protocol used was the ACD ISH DAB Protocol. The RNA unmasking conditions for the tissue consisted of a 15-minute incubation at 95° C. in Bond Epitope Retrieval Solution 2 (Leica Biosystems) followed by 15-minute incubation with Proteinase K which was provided in the kit. Probe hybridization was done for 2 hours with RNAscope probes which were provided by ACD. The probes used for this study were, 2.5 LS TGF-β1 Probe (Catalogue No. 443488); 2.5 LS PRF1 Probe (Catalogue No. 550288); 2.5 LS GZMB Probe (Catalogue No. 550328) and 2.5 LS IL-10 Probe (Catalogue No. 550348). The RNA-ISH assay uses highly specific, branched DNA technology in which signal amplification is implemented to detect target mRNAs within the FFPE tissue section via a series of sequential hybridization steps in which the probe binds to the target mRNA. Subsequent binding of the preamplifier, amplifier and alkaline phosphatase-labelled probe molecules creates a signal amplification structure which can then be visualized with the 3,3′-Diaminobenzidine (DAB) as a chromogen to form a brown dot which can then be visualized using a standard bright field microscope.

### Example 16. In Vivo Assays

For in vivo experiments, bulk T cells (CD4⁺ and CD8⁺) were negatively selected using T cell enrichment RosetteSep Kits with a Ficoll gradient (Stemcell Technologies®). Teff cells were grown in R10 supplemented with 20 IU/ml IL-2. T cells were expanded and transduced as described above for Tconv cells. Tregs were sorted and expanded in OpTmizer™ with 300 IU/ml IL-2 as previously described, for 7 days with beads followed by 7 days of rest. T cell groups were normalized to the same percentage mCherry⁺ on day 14.

For U87 tumor models, mice were injected on day −7 subcutaneously with 6×10⁵ U87 CBG-GFP on the left flank and 6×10⁵ U87-CD19 GBG-GFP on the right flank. 2×10⁶ CAR T cells or the same cell number of UT T cells were injected IV on day 0 with 5 mice per group. In the groups specified recombinant human IL-2 (Peprotech®) was administered IP at 8 μg/mouse 3 times weekly. Tumor burden was regularly monitored using an Ami spectral imaging apparatus and analyzed with IDL software v. 4.3.1 following an IP injection of D-Luciferin substrate solution (30 mg/mL) 2 times a week. Animals were euthanized as per the experimental protocol. U87 tumors were removed on day 14 post CAR injection for paraffin embedding.

For skin xenograft models, human skin was obtained from Massachusetts General Hospital according to IRB protocol. On the same day, skin samples were harvested at the depth of the dermal boundary using a standard Dermablade® (MedLine®) and washed in RPMI media supplemented with 10% FBS. Xenograft procedure was performed as outlined in animal experimental protocol approved by the Institutional Animal Care and Use Committee. Mice were handled using standard aseptic technique. Mouse skin on the dorsum that was approximately twice as large as the graft was removed, immediately followed by creating micro wounds to the fascia using forceps to aid skin engraftment. The human donor graft was then secured to the mouse with silk ligature followed by sterile dressing. After 7-10 days the sutures were removed and antibiotic ointment was applied to the grafts 3 times a week until grafts were fully healed. Once healed, approximately 30-60 days' post-surgery, mice were injected IV (day 0) with 2×10⁶ CAR-Tregs, 2×10⁶ CAR Teff cells or both (4×10⁶ CAR T cells total). In the groups specified IL-2 was administered IP at 8 μg/mouse 3 times weekly. Grafts were photographed 3 times a week. Mice were euthanized on day 14 and tissue was harvested and fixed for paraffin embedding. Graft surface area was measured as from photographs using SketchAndCalc™ software.

### Example 17. Statistical Analysis

Data are presented as means±S.E.M. (standard error of the mean) as stated in the figure legends. Unless otherwise noted, groups were compared using a two-tailed paired student T test. All statistical analyses were performed with Prism® software version 7.0 (GraphPad®).

### Example 18. Study Approval

Healthy donor leukopaks were obtained from the Blood Transfusion Services at Massachusetts General Hospital under an IRB-approved protocol. Human skin was obtained from Massachusetts General Hospital according to an IRB-approved protocol. All subjects provided written informed consent for the use of their discarded tissues for research. Tumor and xenograft procedures were performed as outlined in animal experimental protocols approved by the Institutional Animal Care and Use Committee.

### Example 19. CAR Tregs

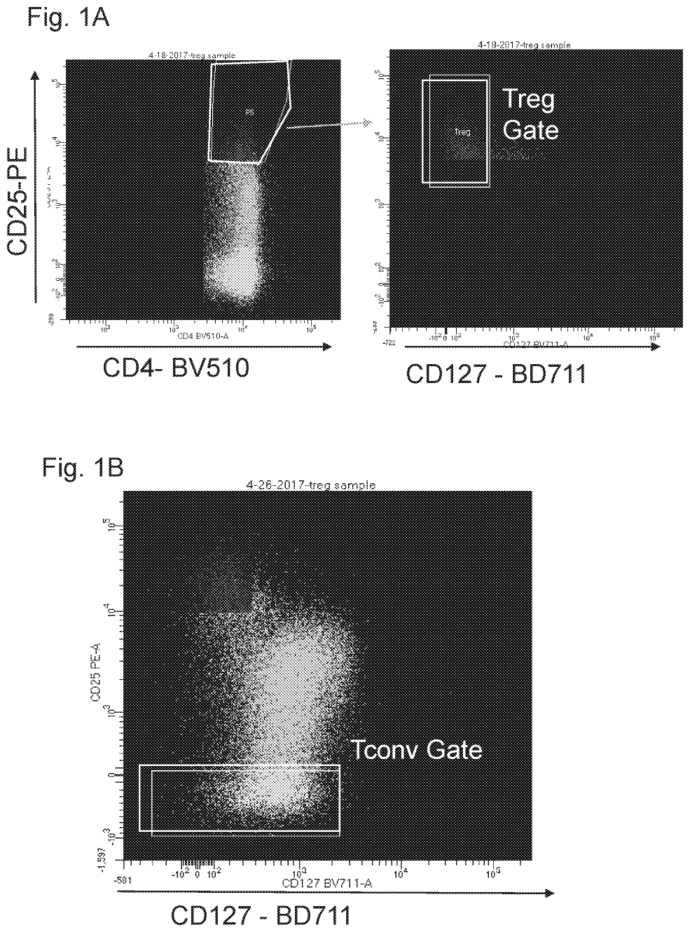

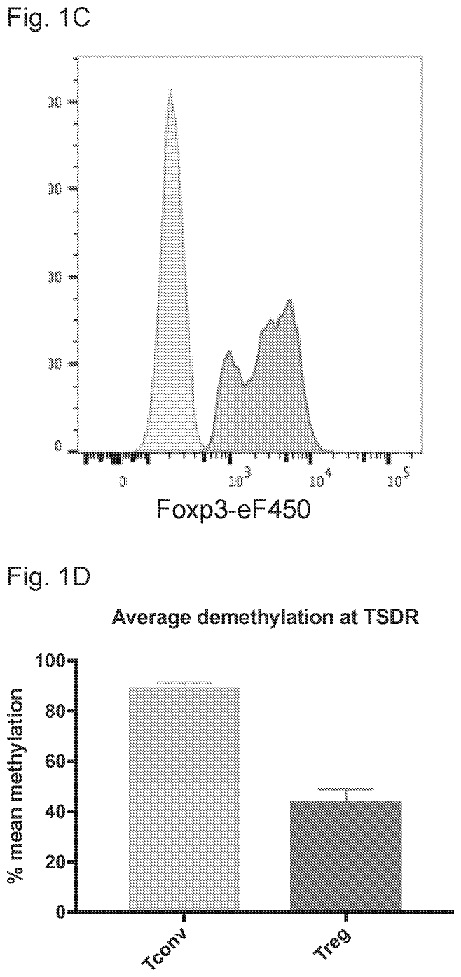

T cell isolation. CD4+ T cells were isolated from human donor leukopaks using RosetteSep™ Human T cell enrichment cocktail. CD4+ T Cells were stained with CD25-PE (5 ul/10e7 cells in 100 μl MACs buffer). MACs selection protocol with anti-PE beads was used to enrich for CD25+ T cells. CD25 depleted cells (flow through from MACs column) were also collected for T cony cells. Cells were stained at 1×10⁷/100 μl in FACs buffer and BD Brilliant Violet buffer with CD4-BV510 (5 μl/100 μl), CD127-BV711 (5 μl/100 μl) and CD8 APC-Cy7 (2.5 μl/100 μl). Cells were incubated for 30 min 4° C. Cells were washed and suspended in sorting buffer. 1 μl DAPI was added before sorting. Tregs were sorted on CD8⁻ CD4^{dim}, CD25^{hi} and CD127^{low} (see FIG. 1A-1D). T cony were sorted by CD8⁻ CD4⁺ CD25^{low}

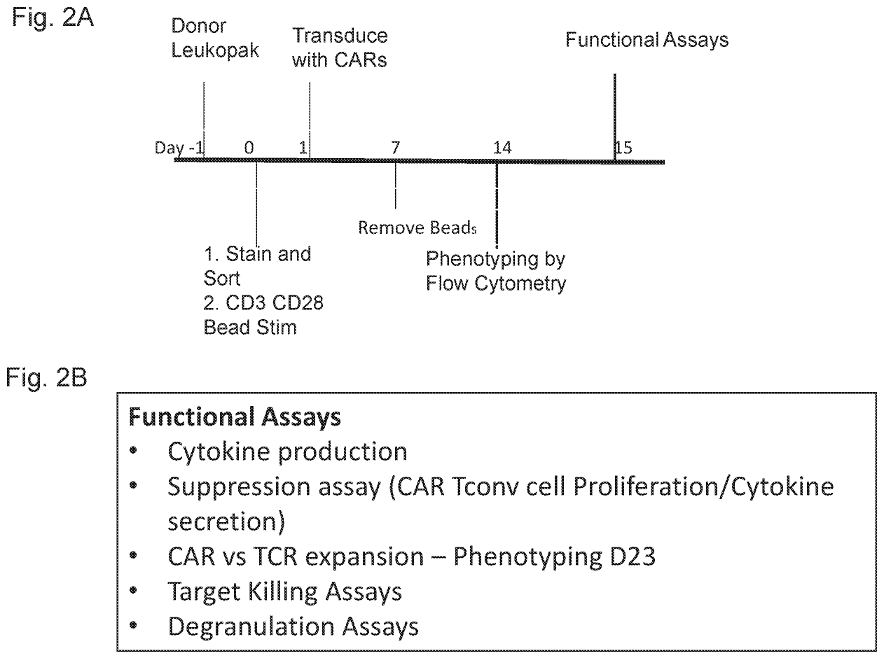

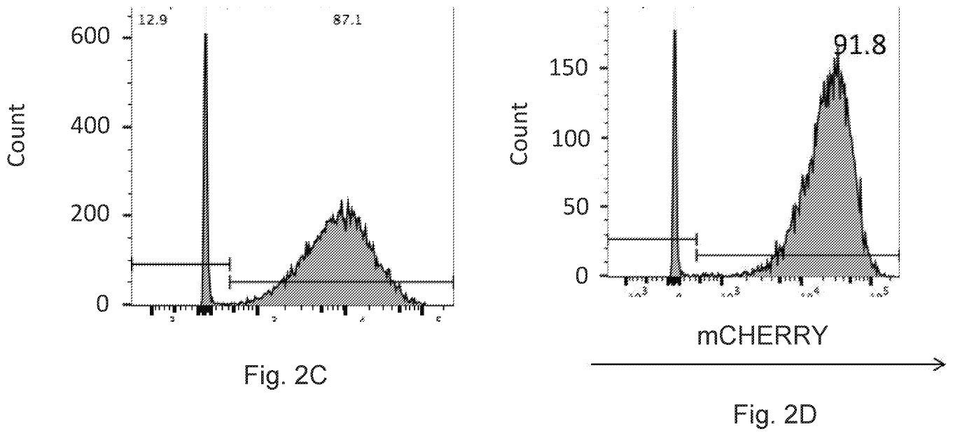

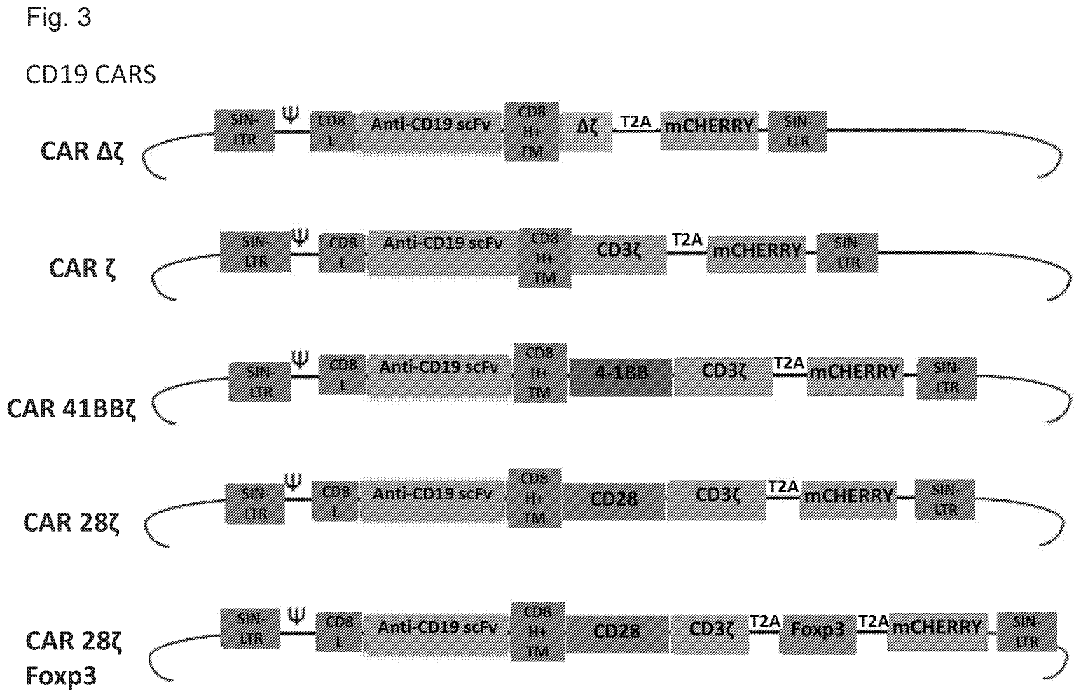

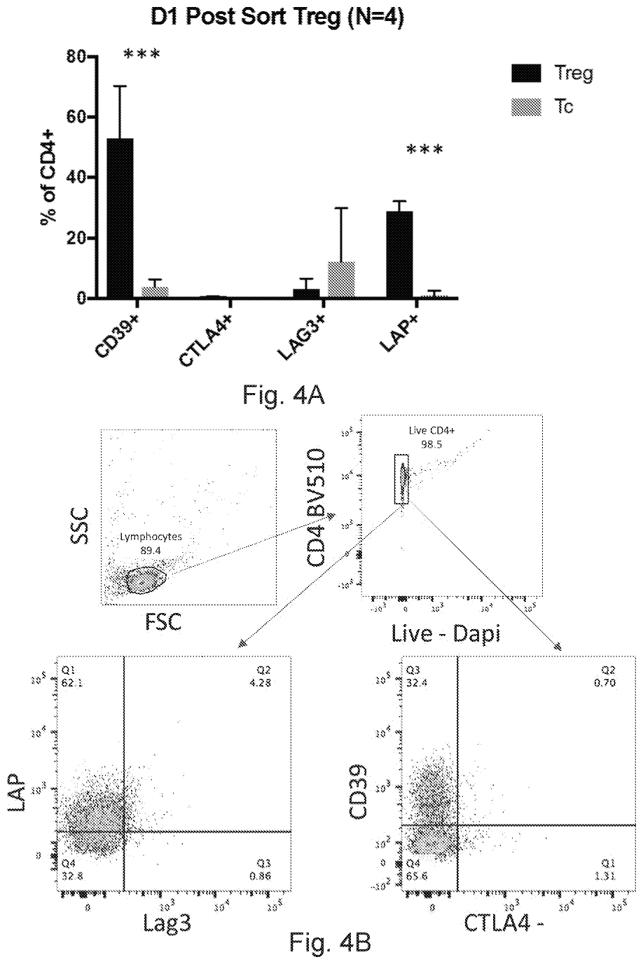

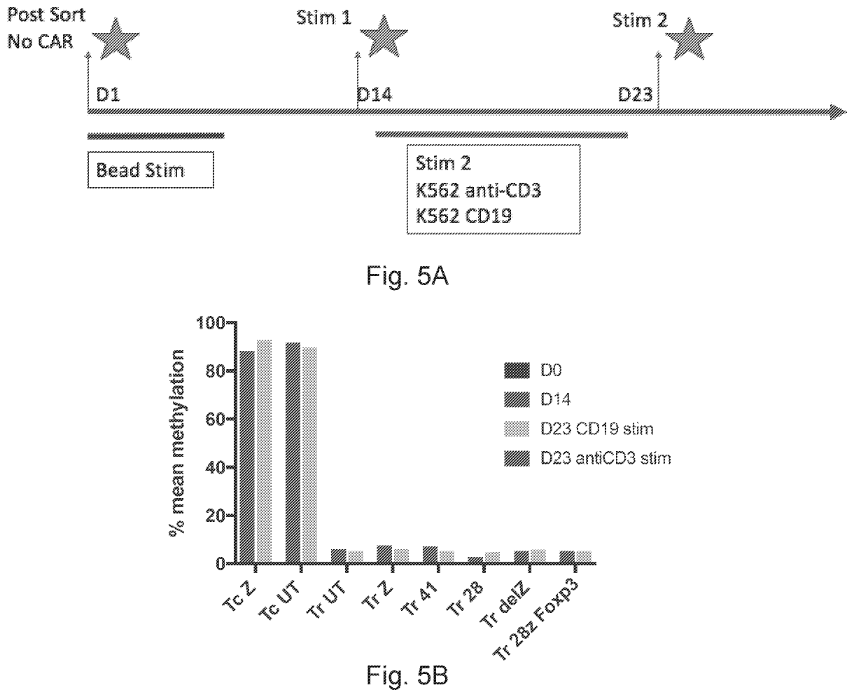

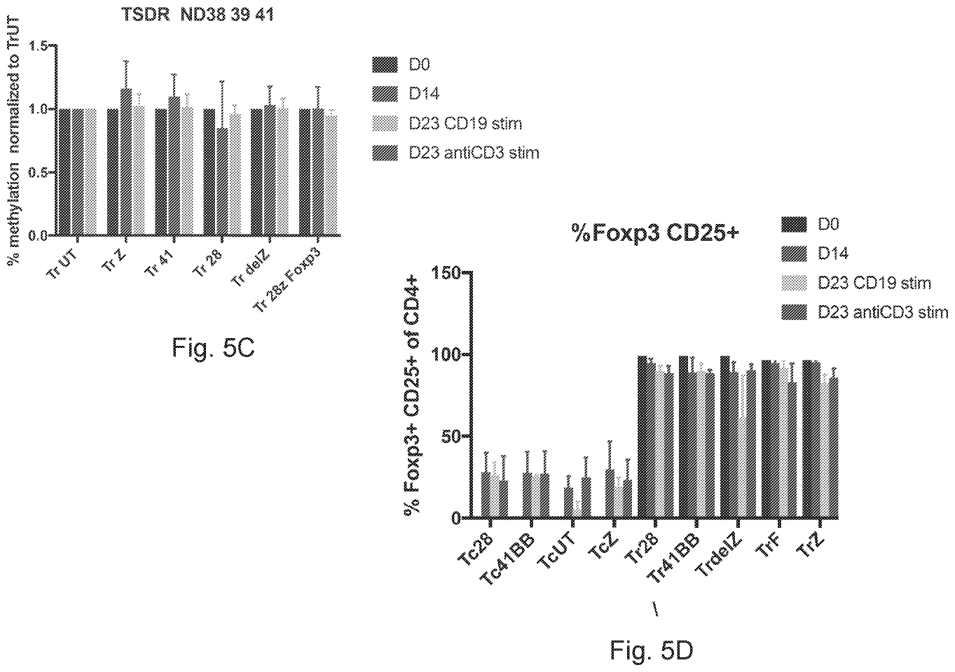

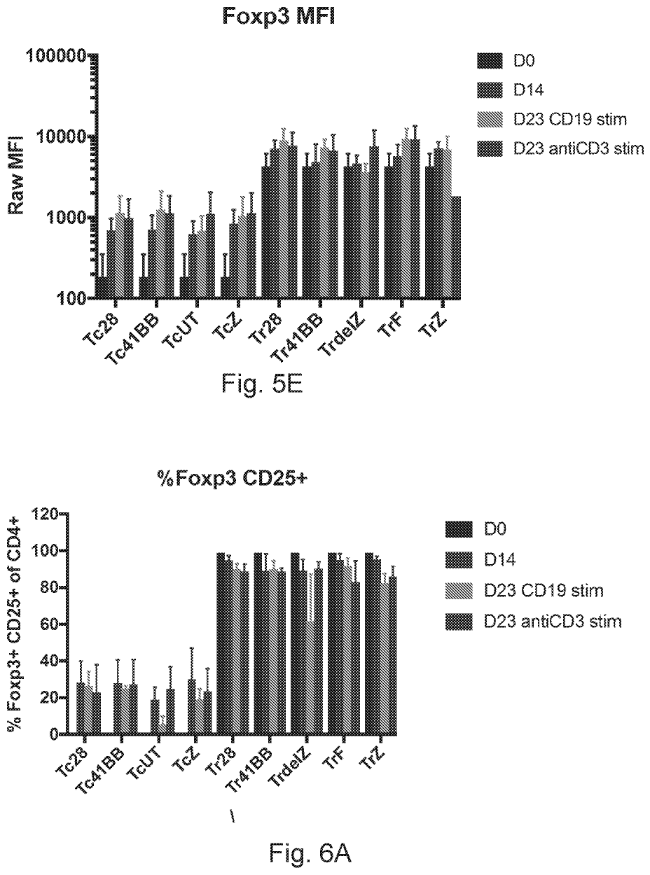

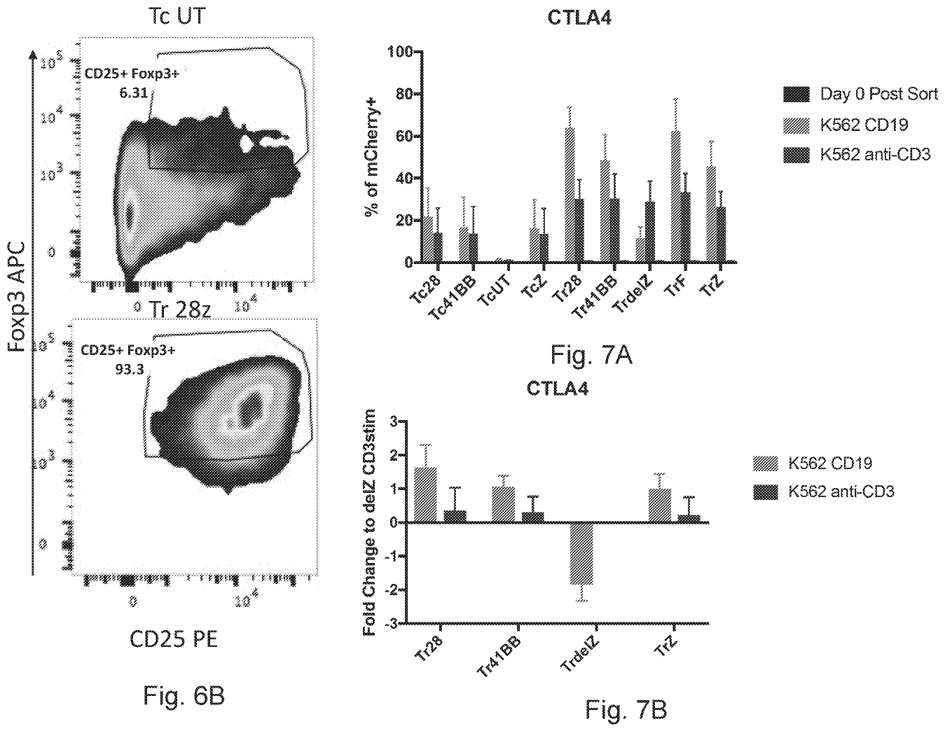

T cells were stimulated, transduced with a number of CAR constructs (FIG. 3) and assayed for phenotype and a number of functional characteristics (FIG. 2A-2D). Foxp3 expression is stable post transduction, bead expansion and CAR stimulation (FIG. 5A-5E, 6A-6B).

The surface phenotypes of the Tregs were examined. CTL4A expression was measured at day 23 (FIG. 7A-7B) and it was found that CTLA4 is upregulated on CAR Tregs and CAR Tconv post stimulation. There is a higher % of CTLA4+ cells in CAR Tregs than T cony cell. CTLA4 is upregulated on CAR Tregs stimulated with antigen vs CD3.

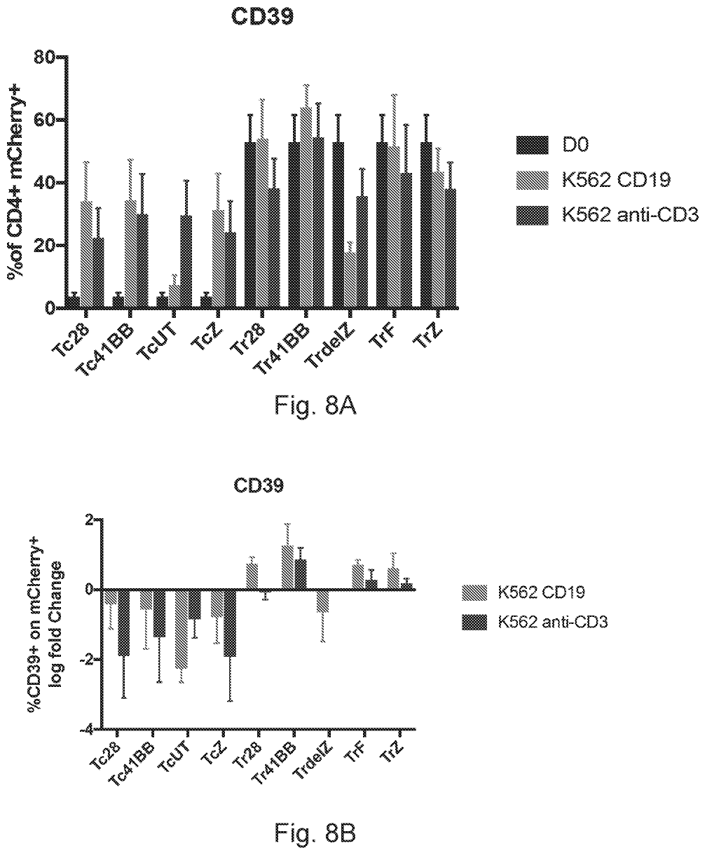

CD39 (Ectonucleoside triphosphate diphosphohydrolase-1) expression was also examined (FIGS. 8A-8B) and it was found that CD39 maintains its surface level on CAR Tregs after stimulation, CD39 increases its surface expression in Tconv cells after stimulation, and CD39 is expressed at higher levels on 4-1BB CAR Tregs than other Tregs.

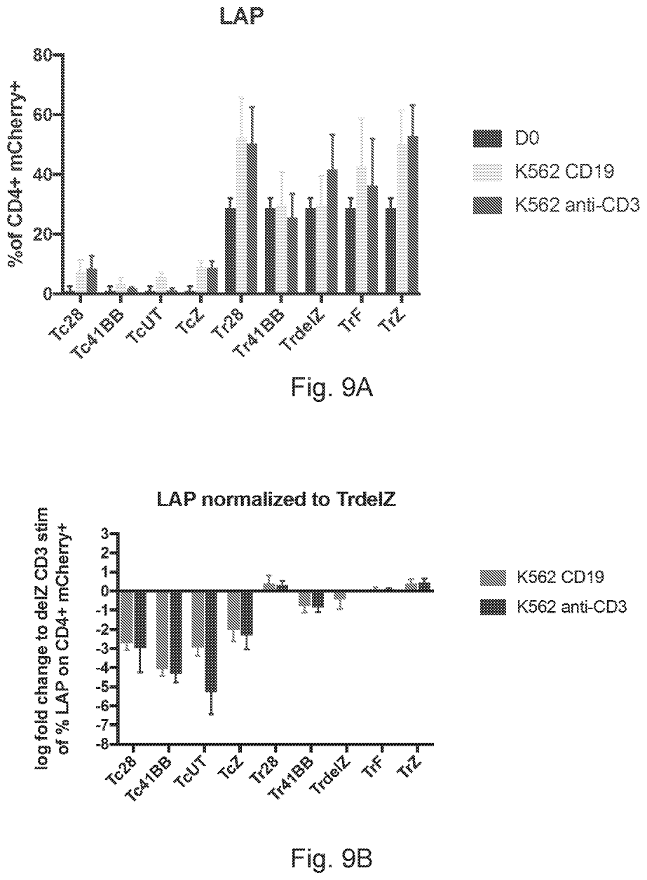

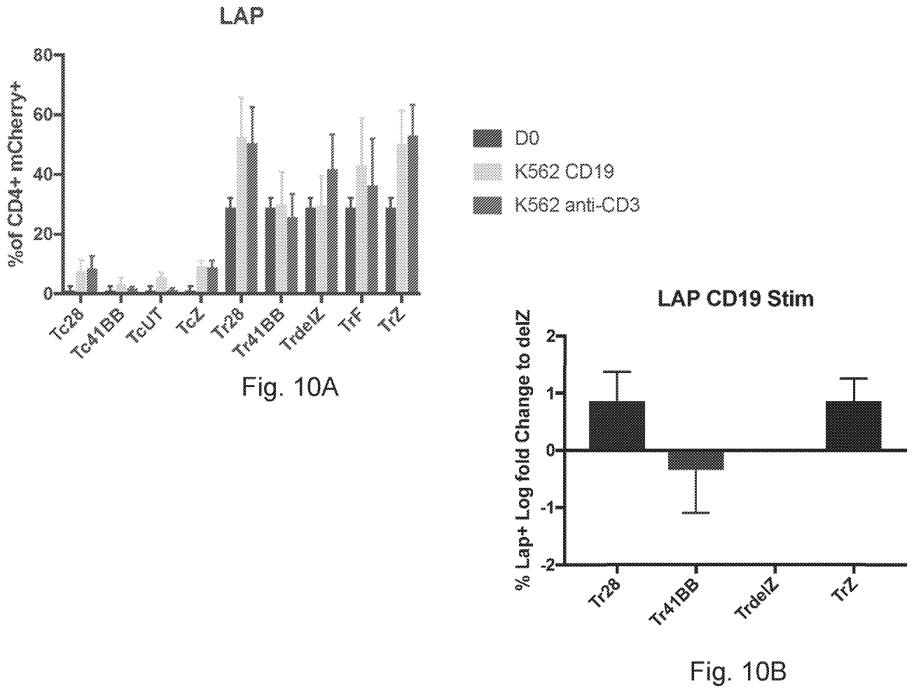

LAP maintains its surface expression on Tregs (FIG. 9A-9B). LAP is expressed on Tregs, expressed at very low levels on Tconv cells vs Tregs, and 41BBz CAR Treg LAP does not increase after CD3 or CD19 stimulation whereas TrZ and Tr28z do increase. On day 23, Tregs were found to maintain LAP surface expression (FIG. 10A-10B). LAP is expressed on Tregs and at very low level on Tconv cells vs Tregs. 4-1BBζ CAR Treg LAP does not increase after CD3 or CD19 stimulation whereas Trζ and Tr28ζ do increase. 4-1BBζ CAR has significantly lower LAP expression than 28ζ and ζ.

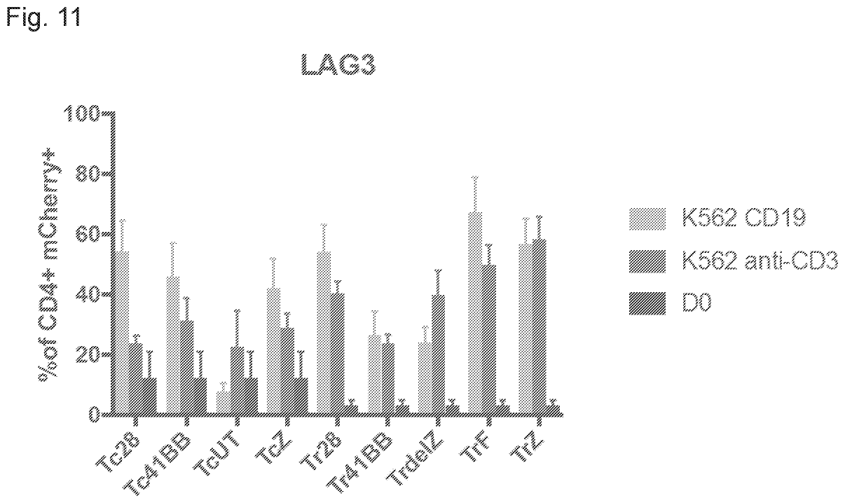

LAG3 surface expression increases with CAR stimulation (FIG. 11). There is no difference between Tconv and Treg LAG3 expression and LAG3 is increased following T cell expansion and K562 activation.

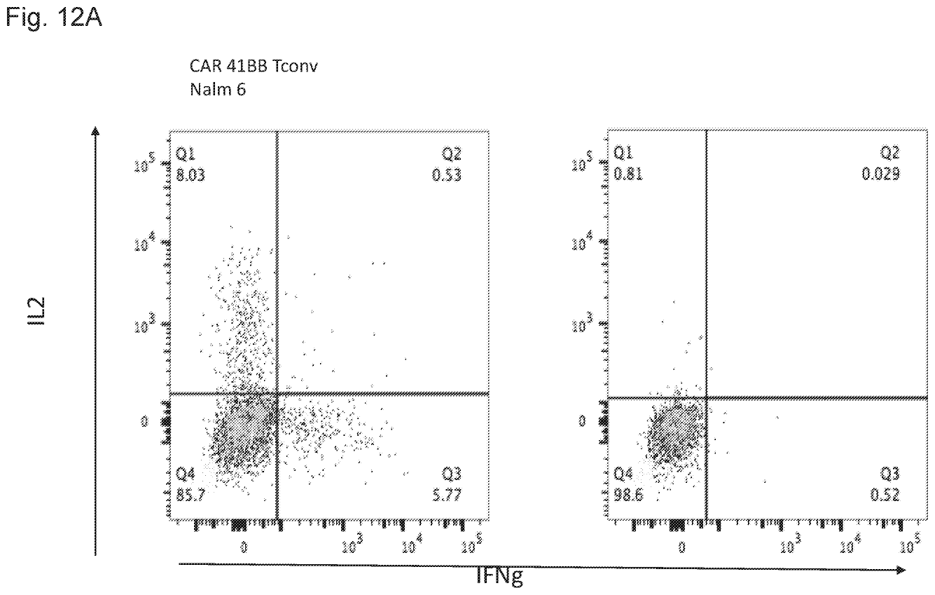

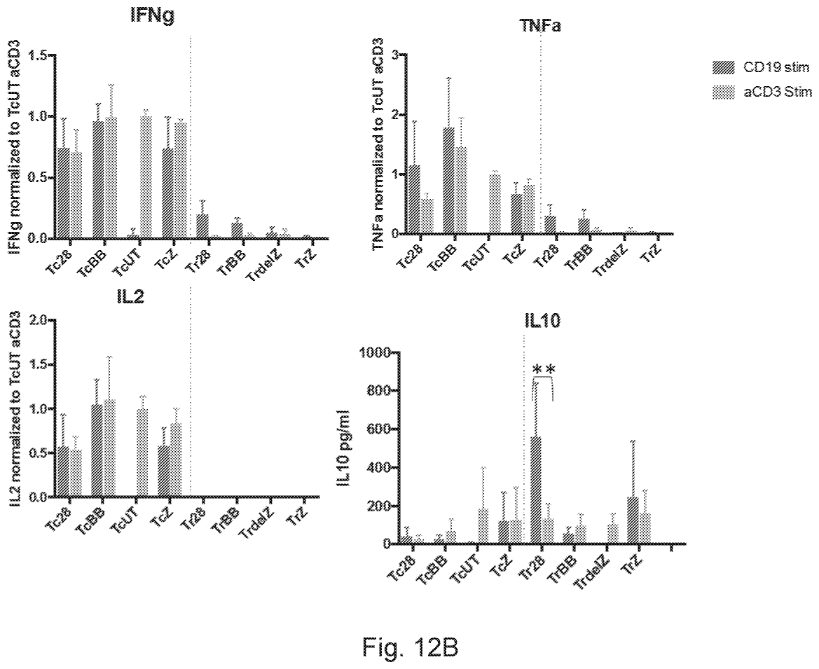

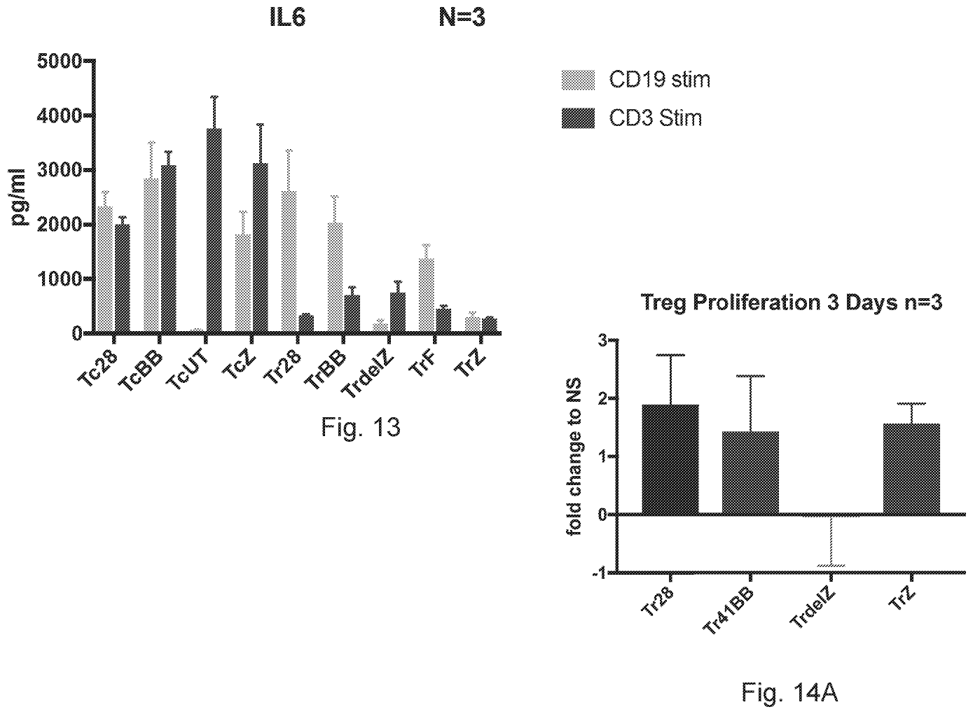

CAR Tregs do not make inflammatory cytokines in response to antigen stimulation, but do produce IL-10 (FIG. 12B). CAR Tregs with costimulation after CD19 CAR stimulation do make TNFα and IFNγ (FIG. 12A). CAR Tregs demonstrate increased IL-6 expression after stimulation (FIG. 13).

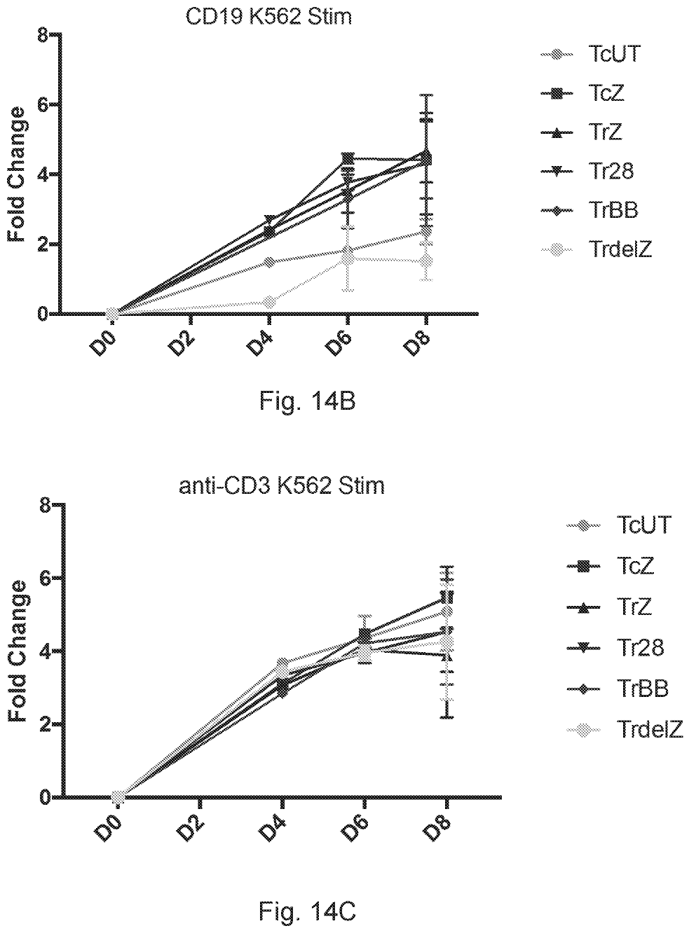

CAR Tregs were demonstrated to proliferate in response to antigen stimulation with IL-2 (FIGS. 14A-14C).

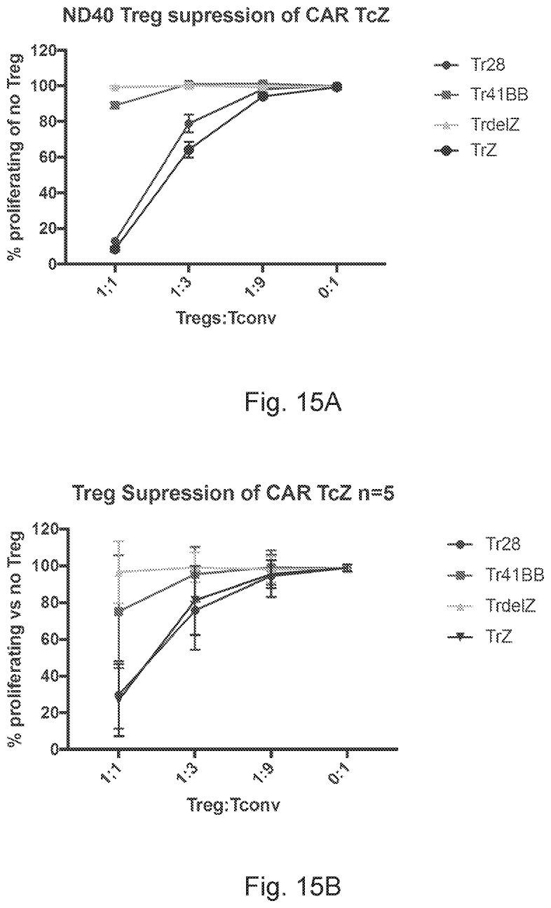

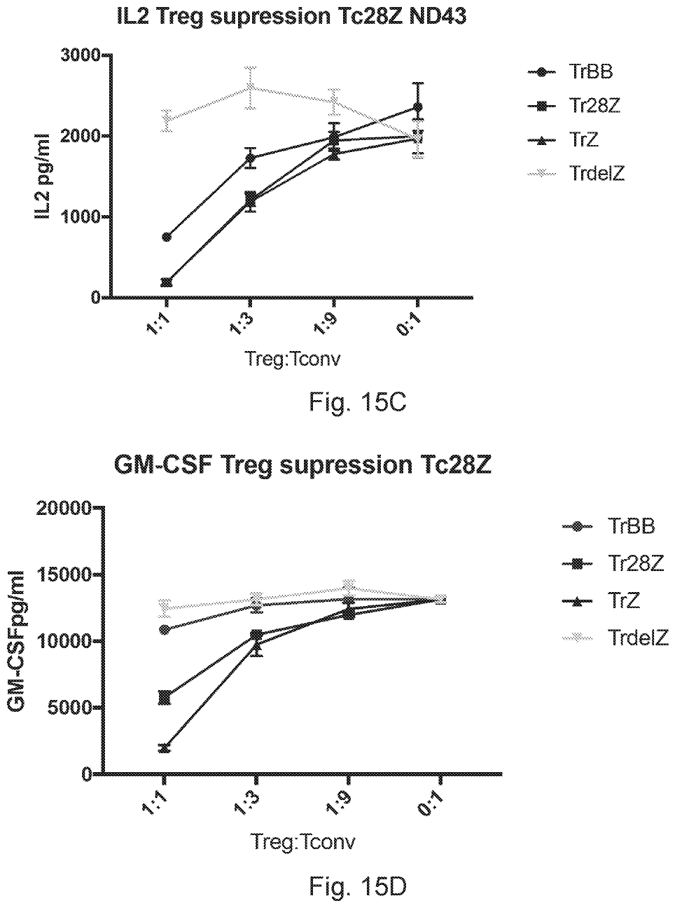

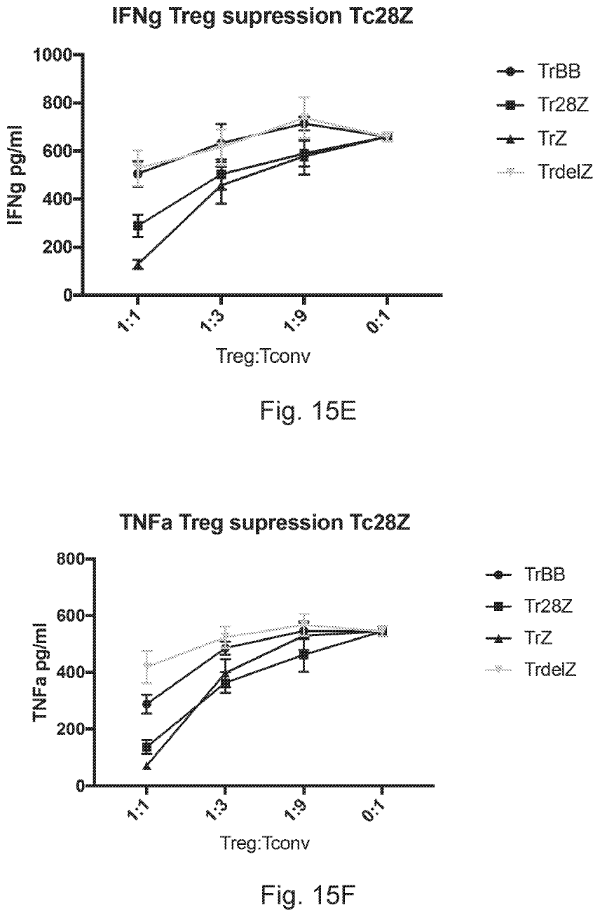

Tregs have suppressive function. 4-1BBζ Treg does not suppress proliferation or cytokine secretion of CART cony cells as well as Trζ and Tr28ζ (FIGS. 15A-15F).

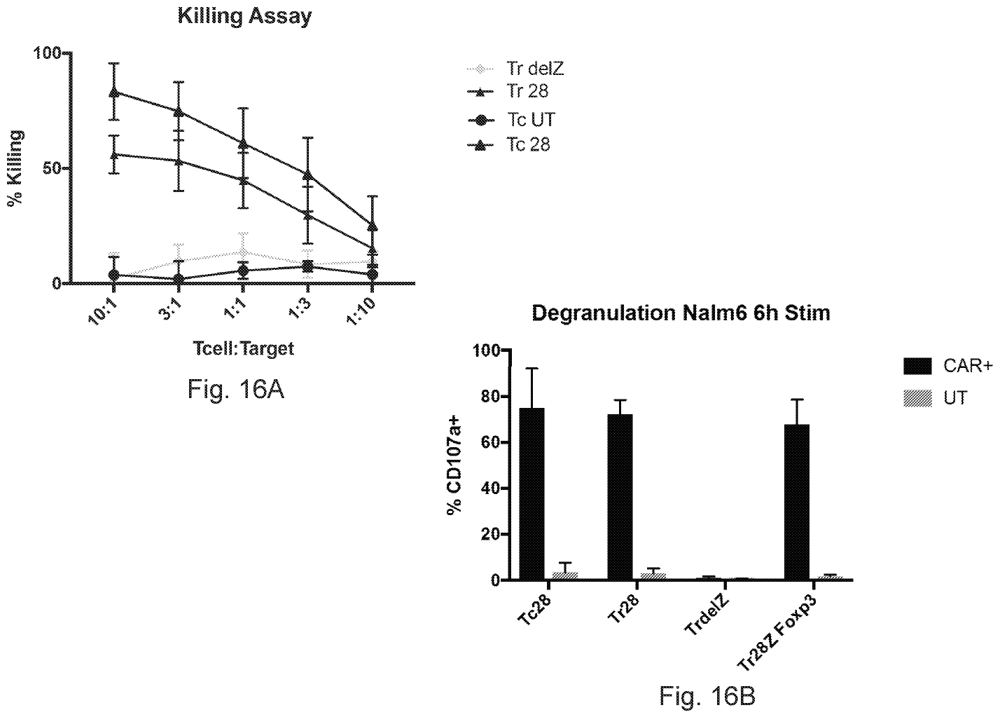

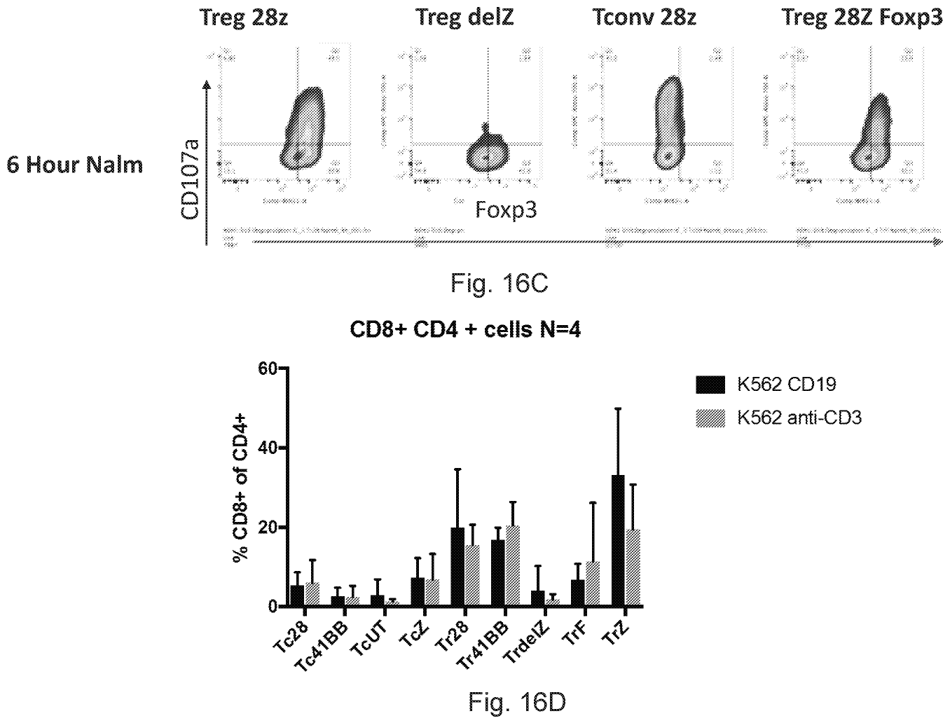

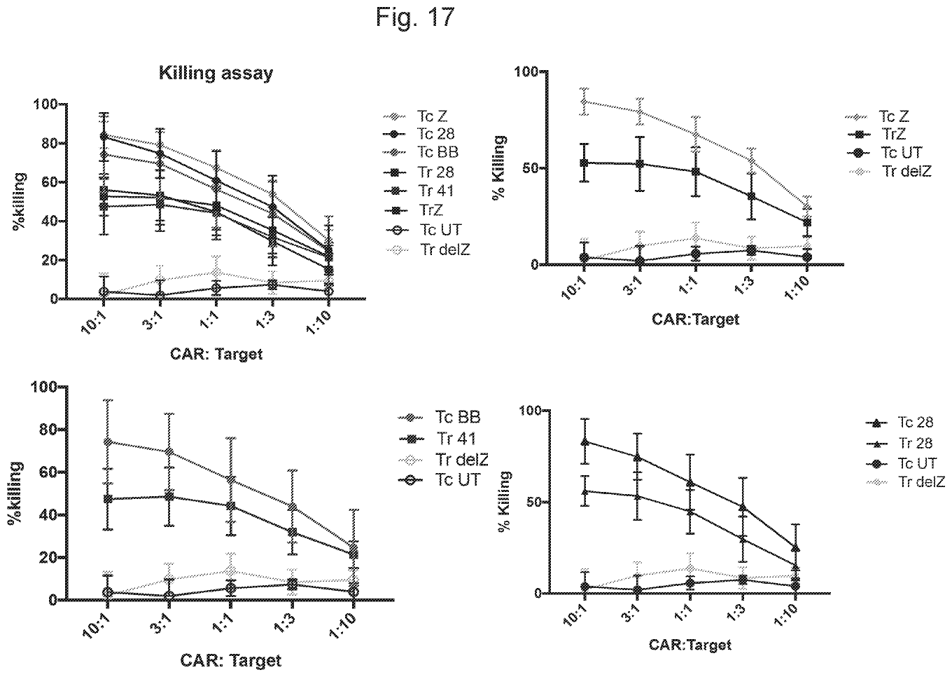

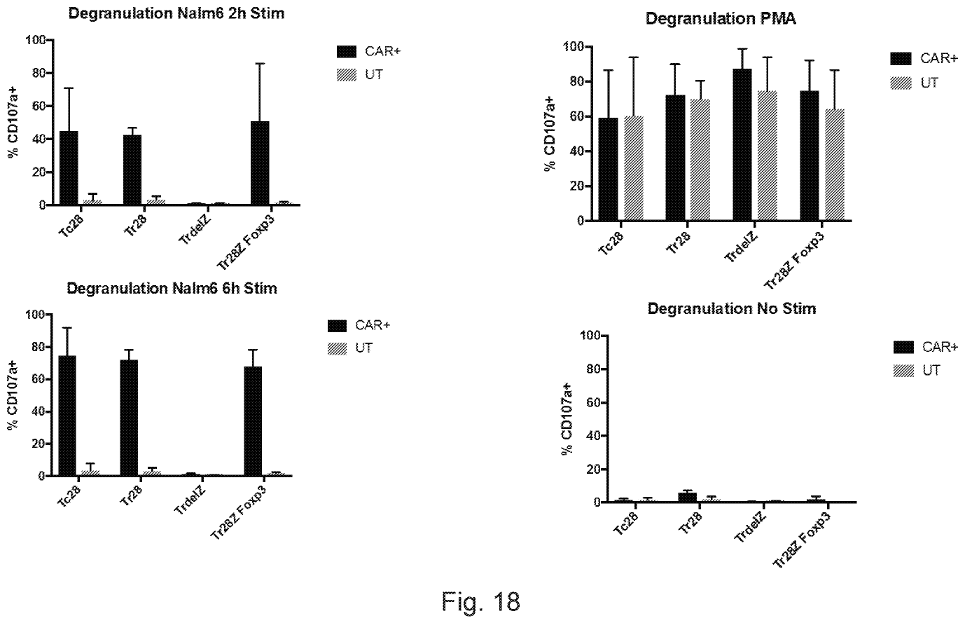

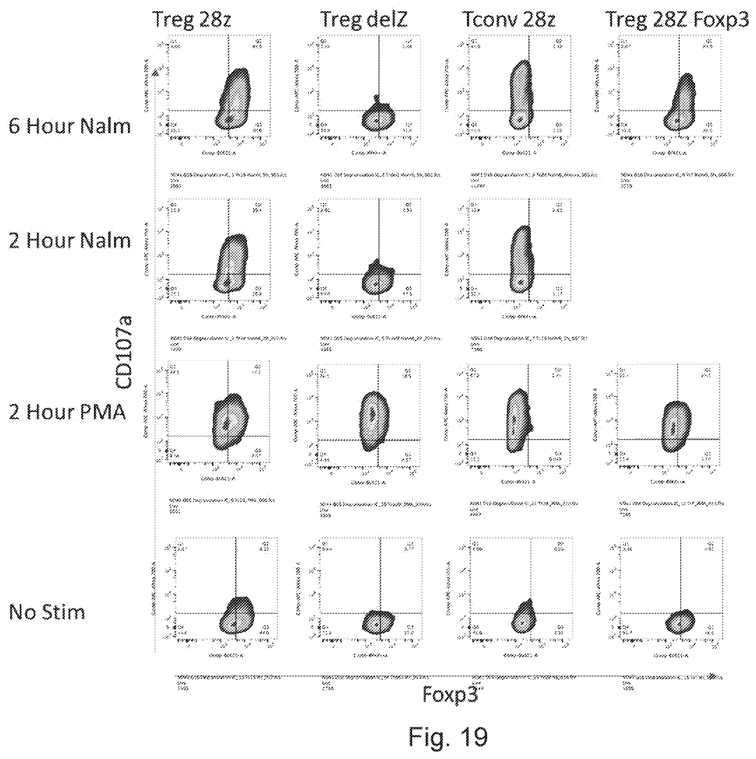

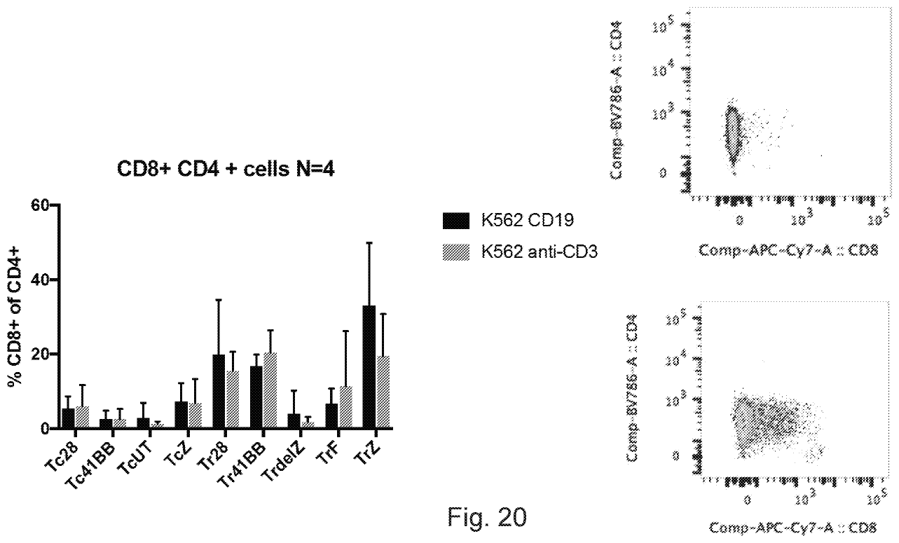

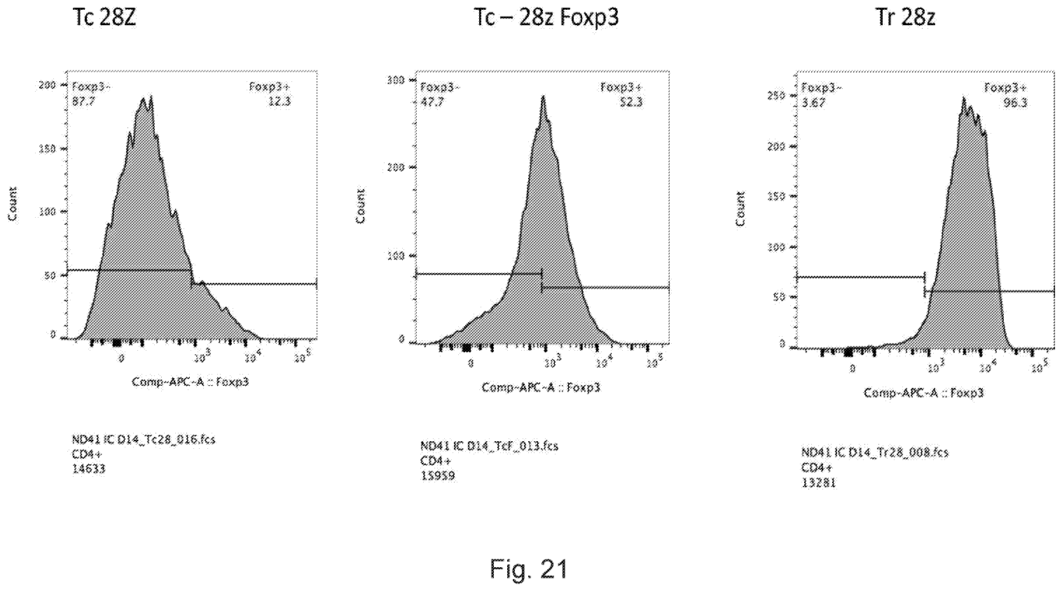

Stable Foxp3+ Tregs still kill target cells (FIG. 16A-16D). Tregs kill but at a lower rate than T cony cells (FIG. 17, 18). CD8 is upregulated in all CD4 CAR T cells but to a greater extent in the CAR Tregs (FIG. 20).

It is demonstrated herein that CAR Tregs maintain Foxp3 expression and express equal or higher levels of Treg specific markers than UTD/CARdelZ Tregs. CAR Tregs do not make inflammatory cytokines in response to antigen and do produce IL-10. CAR T stimulation results in greater IL-10 secretion than TCR stimulation in CAR 28c CAR Tregs inhibit Teff cell proliferation and cytokine secretion in an antigen specific manner. CD2K and CAR Tregs are better at suppressing proliferation than 4-1BB CARs but all CARs are equally stable. CAR Tregs can degranulate and kill target cells that they are meant to protect and the transgenic expression of foxp3 cannot overcome this degranulation.

Described herein are methods to ensure that Tregs are indeed Tregs. Degranulation assays demonstrate that the degranulating cells express Foxp3.

### Example 20. Isolation of Tregs and Transduction with CARs Bearing Different Signaling Domains

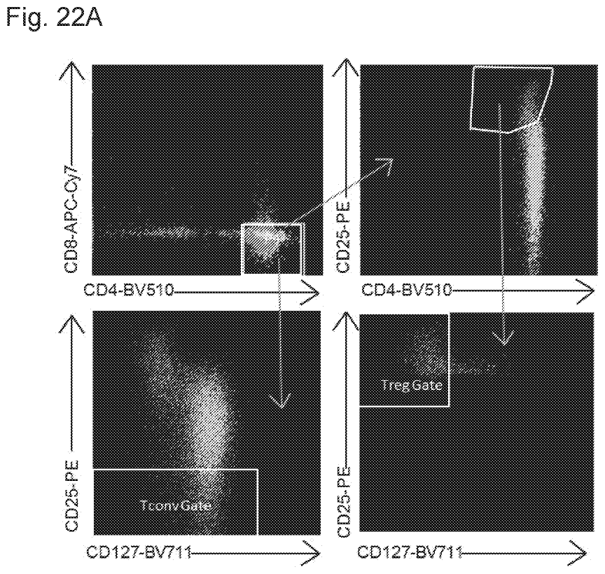

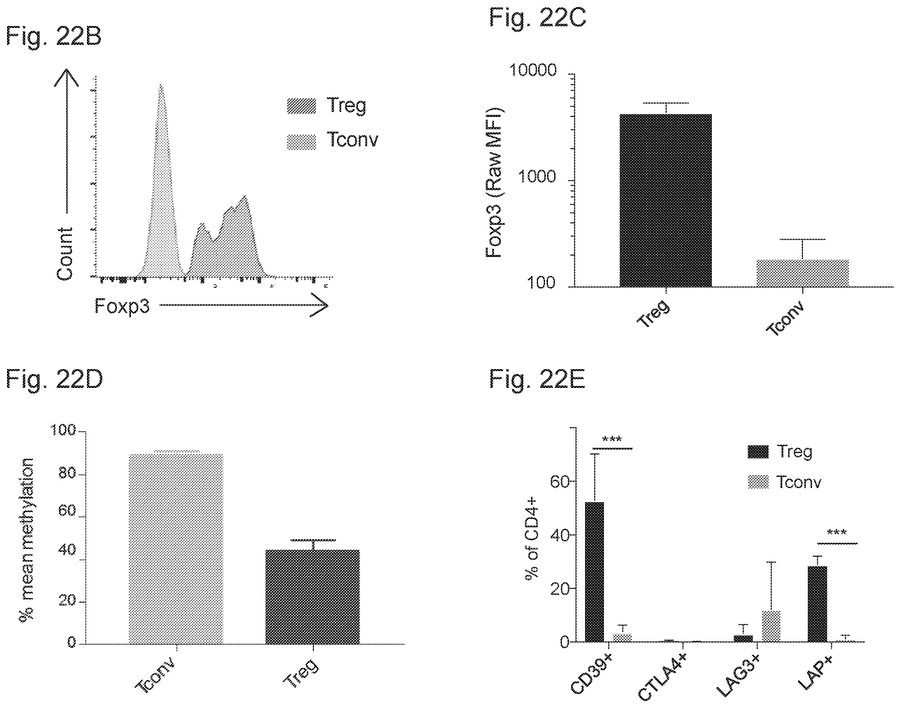

Healthy donor leukopaks were purchased from the MGH blood bank under an IRB-approved protocol and the CD25⁺ population was enriched by magnetic selection. Tregs were further purified by FACS sorting based on expression of the surface markers CD4⁺, CD25^{hi} CD127^{low} (FIG. 22A). In parallel, CD4⁺ CD25^{low} T conventional (Tconv) cells were also sorted to use as controls from the same donor in each experiment (FIG. 22A). The sorted human Tregs were confirmed to express intracellular Foxp3 (FIGS. 22B and 22C) and were de-methylated at the Treg specific demethylation region (TSDR) of the X chromosome (FIG. 22D). Sorted Tregs also expressed higher levels of the phenotypic markers that differentiate resting Tregs from conventional T cells (Tconv), including CD39 and the latency associated peptide (LAP), which is part of the latent TGFβ complex (FIG. 22E). Surface expression of CTLA4 and LAGS was undetectable, as expected for non-activated Tregs (FIG. 22E).

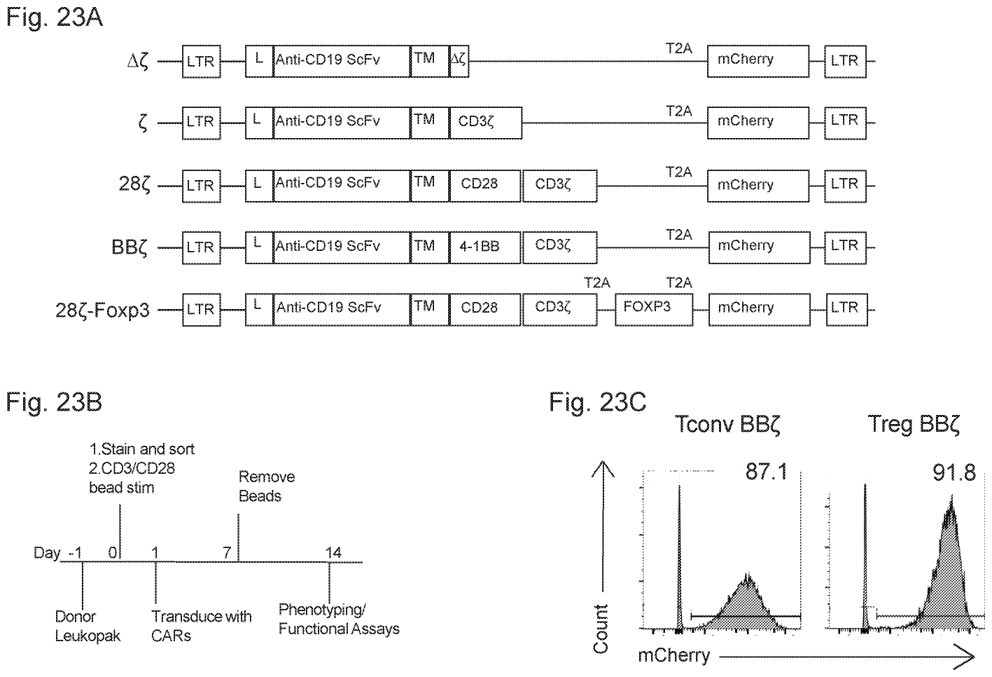

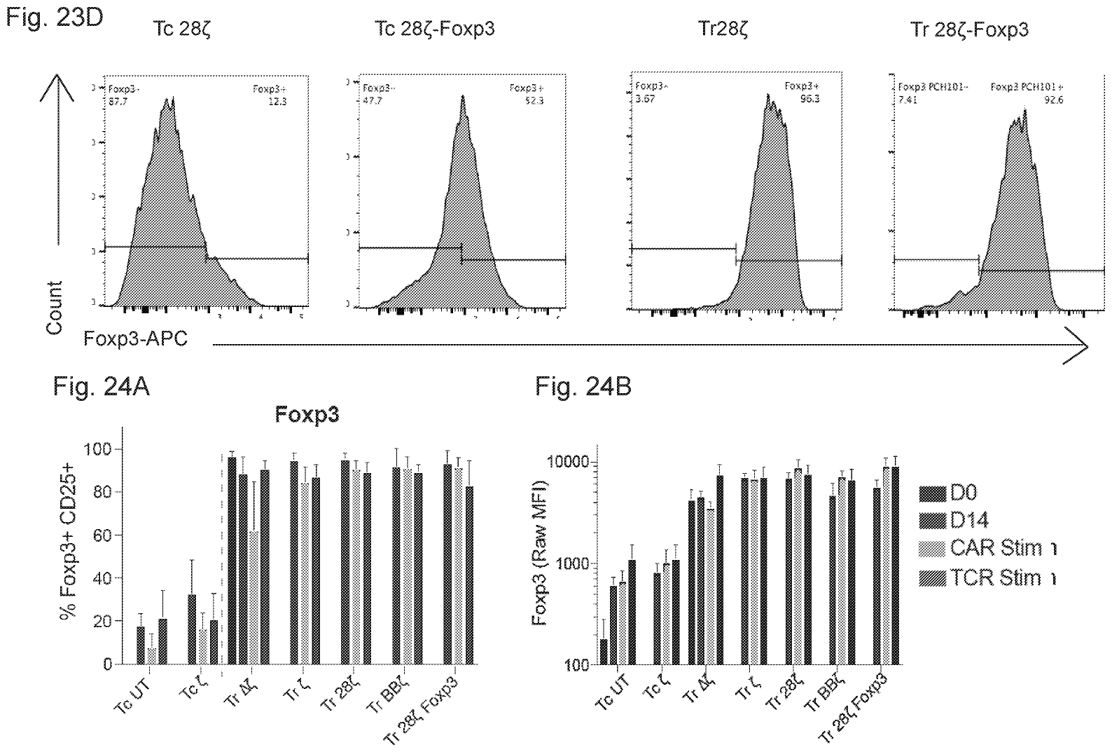

Five different CAR constructs were synthesized in a lentiviral backbone (FIG. 23A):

(1) a control CAR construct that contained a truncated, non-signaling CD3ζ chain (Δζ),

(2) a first-generation CAR that contained only a CD3ζ signaling domain (ζ),

(3) a second-generation CAR with a 4-1BB costimulatory domain (BBζ),

(4) a second-generation CAR with a CD28 costimulatory domain (28ζ), and

(5) a second-generation 2ζ CAR that expressed transgenic Foxp3 after a cleavable T2A element (28ζ-Foxp3).

All CARs had the same single chain variable fragment (scFv) against CD19 with identical CD8 hinge and transmembrane domains. An mCherry fluorescent reporter gene was included downstream of the CAR construct after a T2A element to facilitate evaluation of CAR transduction. Immediately after sorting Tregs and Tconv cells, the cells were activated with anti-CD3/anti-CD28 expander beads, and 24 hours later, transduced with lentiviral vector carrying the CAR constructs. CAR-Tregs were expanded with beads for 7 days followed by bead removal, then rested for another 7 days in media containing 300 IU/ml rhIL-2. CAR-Tregs were phenotyped and used in various functional assays ˜2 weeks from initial isolation (FIG. 23B). Tconv and Treg cells showed similar transduction efficiencies with CAR vectors at the same multiplicity of infection (MOI) (FIG. 23C). We confirmed that T cells modified with the CAR-28ζ-Foxp3 construct did indeed express transgenic Foxp3, even when transduced into Tconv cells (FIG. 23D).

### Example 21. Phenotype and Foxp3 Stability of CAR Tregs

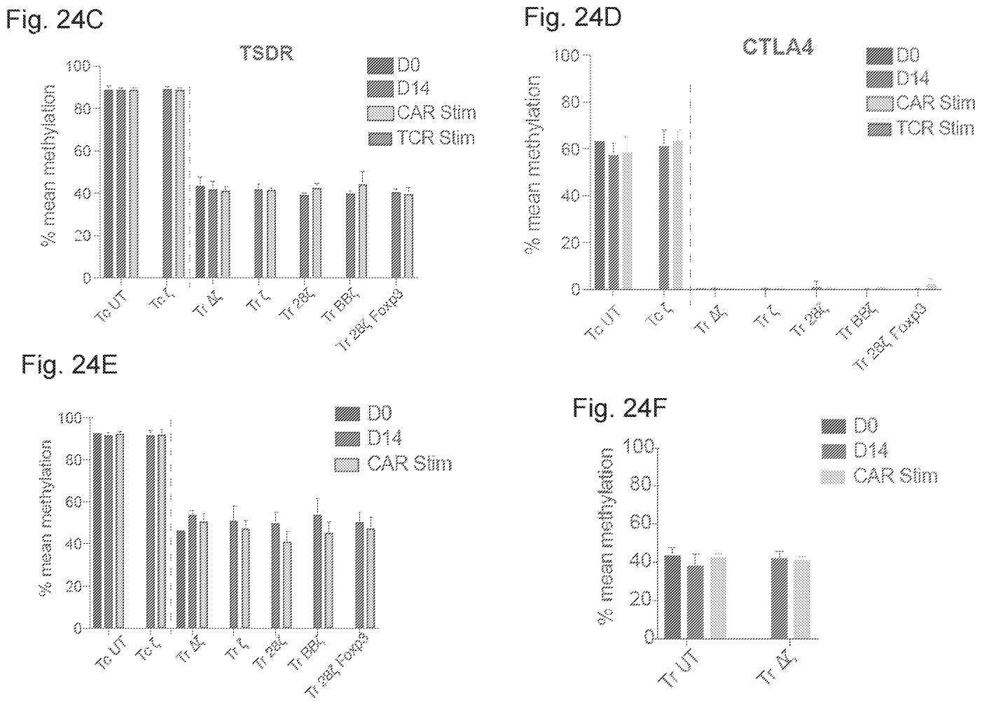

CAR-modified Tregs were analyzed for the expression of Foxp3 and the methylation status of the Treg-specific demethylation region (TSDR), CTLA-4 promoter, and Helios promoter, with the expectation that Tregs would maintain high expression of Foxp3, and remain demethylated at the TSDR and Helios and CTLA4 promoter loci. CAR Tregs were analyzed at day 14, a time point where CAR-Tregs would presumably be harvested/infused (day 14), and also analyzed following antigen encounter, either through their TCR or their CAR (day 23). The antigen encounter stimulation was performed by a 9-day co-culture of CAR-Tregs with irradiated K562s transduced to express either membrane-bound anti-CD3 scFv (OKT3) to stimulate the TCR or CD19 to stimulate the CAR, respectively. These “rested” time points were chosen rather than immediately following bead activation or antigen encounter because many Treg-associated markers, including both CD25 and Foxp3, are expressed on activated human Tconv cells (Kmieciak et al., J. Transl. Med. 7:89, 2009). Intracellular Foxp3 staining demonstrated that CAR-Tregs remain Foxp3⁺ irrespective of the CAR costimulation domain. This expression was maintained at day 14 and following antigen stimulation through their CAR or TCR at day 23 (FIG. 24A, FIG. 24B). Similarly, demethylation of the TSDR locus remained stable after isolation (day 0), initial transduction (day 14) and following antigen stimulation through the CAR (day 23) (FIG. 24C). It was also observed that the mean methylation of CTLA4 (FIG. 24D) and IKZF2 (Helios, FIG. 24E) locus was lower in all CAR-Tregs compared to CAR-Tconv cells at day 0 and remained stable through transduction (day 14) and re-stimulation (day 23), and that the methylation status of these loci was independent of the CAR construct. Untransduced (UTD) Tregs behaved identically to Δζ CAR Tregs, such as TSDR methylation status (FIG. 24F).

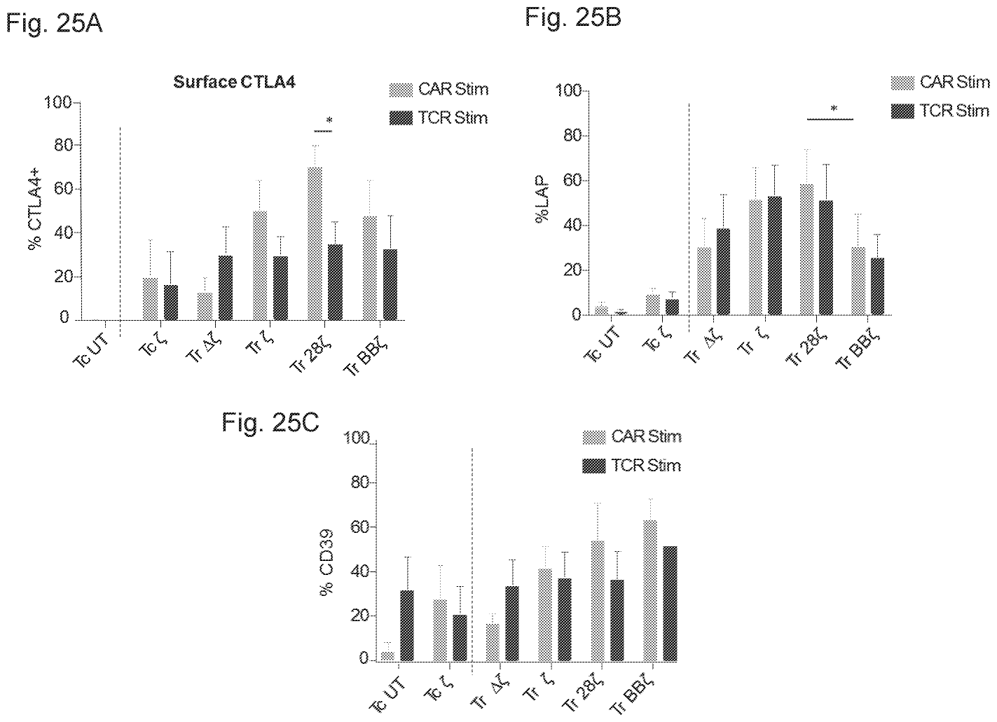

CAR-Tregs were analyzed for surface expression of classic Treg functional markers CTLA4, LAP and CD39 following stimulation through their CAR or TCR. It was observed that antigen stimulation through CD28-based CARs induced significantly higher CTLA4 expression than signaling though the TCR in CAR-Tregs (FIG. 25A), as had been previously described in HLA-A2-directed 28-based CAR Tregs (MacDonald et al., J. Clin. Invest. 2016; 126(4):1413-1424). There were no statistically significant differences in CTLA4 expression when signaling through first-generation or BBζ second-generation CARs compared to TCRs in Tregs (FIG. 25A). It was also observed that 28ζ CAR-Tregs had higher LAP expression than BBζ CAR-Tregs when stimulated through either their CAR or TCR (FIG. 25B). CD39 expression was less variable across different kinds of CAR-Tregs (FIG. 25C), and there was no significant difference in stimulation through the CAR or TCR. From these results, it appears that the expression of a CAR and the type of costimulatory domain does not affect Foxp3 stability or the methylation status of CTLA4 and IKZF2 promotors. However, transduction with CARs can impact the expression of activation-dependent Treg phenotypic surface markers CTLA4 and LAP, with 28ζ increasing the expression of both of these markers compared to both BBζ and Δζ in resting CAR-Tregs.

### Example 21. Activation of CAR Tregs Through CAR and TCR

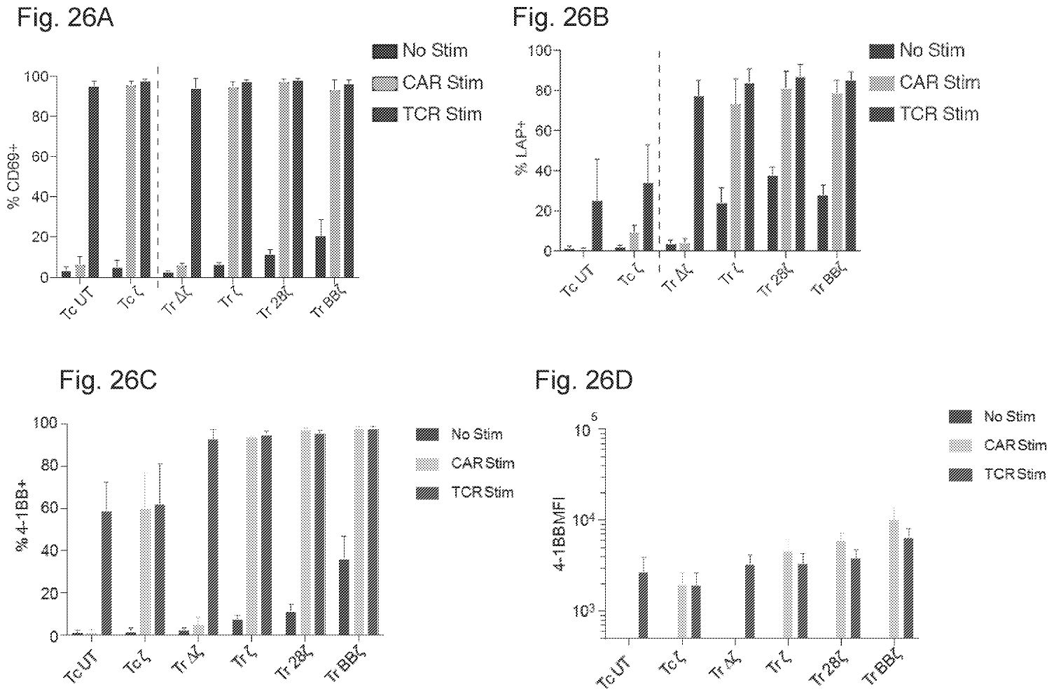

Tregs need to be activated to become suppressive (Thornton et al., J Exp Med. 1998; 188(2):287-296; Takahashi et al., Int Immunol. 1998; 10(12):1969-1980). Like Tconv cells, functional Tregs upregulate CD69 when activated. However, unlike Tconv cells, Tregs do not secrete inflammatory cytokines but instead secrete suppressive cytokines such as IL-10 (Maynard et al., Nat Immunol. 2007; 8(9):931-941). Tregs also upregulate specific functional markers such as LAP following activation. LAP is a protein that associates with TGFβ, a pleotropic cytokine know to inhibit Teff cell proliferation and IL-2 secretion (Kehrl et al., J Exp Med. 1986; 163(5):1037-1050). LAP expression on Tregs correlates with TGFβ secretion (Tran et al., Blood. 2009; 113(21):5125-5133). The expression of activation markers in Tregs transduced with various CARs after stimulation was measured. CAR-Tregs were activated through their CAR or TCR for 20 hours in vitro, using irradiated K562 cells as above. Like CAR-Tconv, all CAR-Tregs upregulated CD69 in response to both CAR and TCR activation, except untransduced (UT) Tconv and Δζ CAR-Tregs which were not activated by K562-CD19, as expected (FIG. 26A). In contrast to CD69, only Tregs and not Tconv cells expressed high amounts of LAP after CAR or TCR activation (FIG. 26B). CAR-Tregs expressed higher amounts of LAP at rest compared to control Tregs (Δζ and UT), suggesting that there is a mild constitutive CAR signaling effect in the absence of antigen stimulation. Expression of 4-1BB is also an activation marker in Tconv and in Treg cells (Nowak et al., Front. Immunol. 2018; 9:199), and 4-1BB was upregulated after stimulation through Treg CARs or TCRs. Notably, BBζ CAR-Tregs expressed higher levels of surface 4-1BB both at baseline and after activation (FIGS. 26C and 26D).

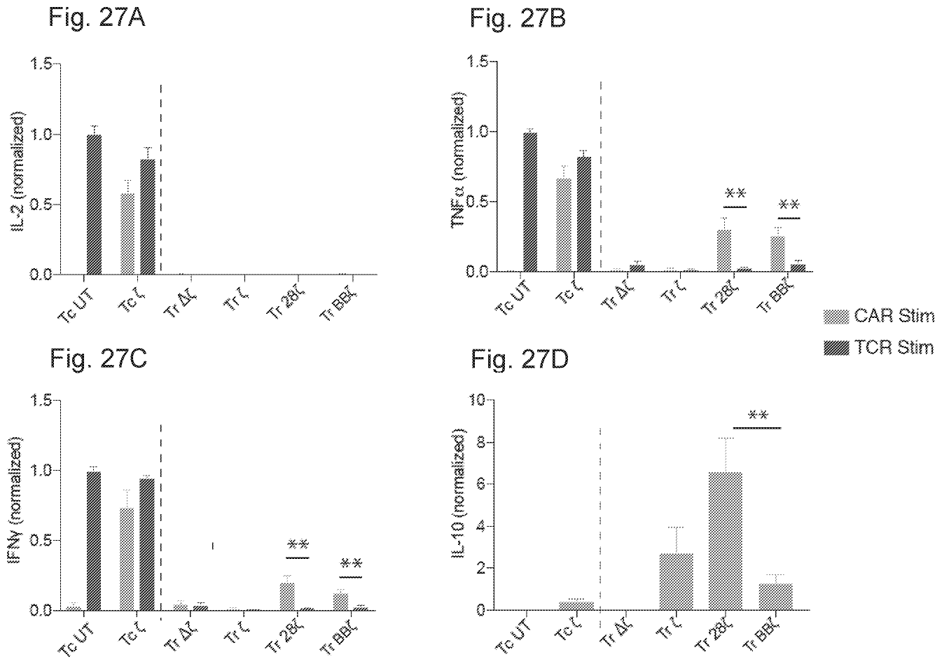

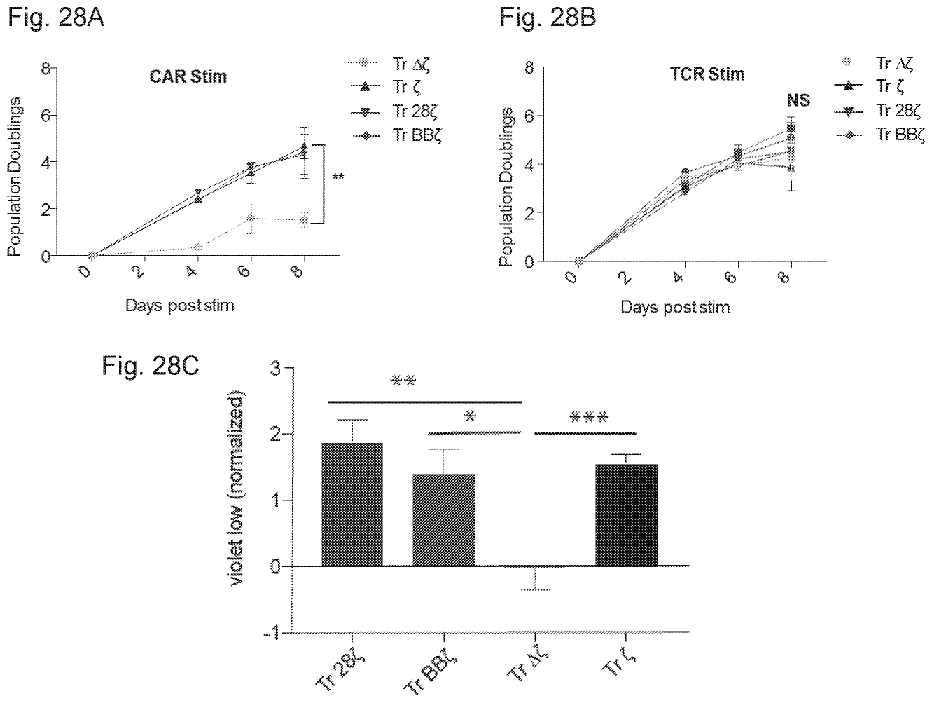

The cytokine profiles in the supernatants of CAR-Tregs activated for 24 hours with K562-CD19 or K562-OKT3 were measured next. CAR-Tconv cells produced high amounts of inflammatory cytokines (IL-2, TNFα, IFNγ) in response to CAR activation, whereas Tregs produced minimal if any inflammatory cytokines (FIG. 27A-27C). Cytokines were not detected in resting Tconv or Treg supernatants (data not shown). However, it was observed that CAR stimulation in 28ζ and BBζ CAR-Tregs induced low but significant levels of IFNγ and TNFα compared to the first-generation CAR-Tregs or CAR-Tregs activated with anti-CD3. It was also found that CAR-Tregs produced higher amounts of IL-10 in response to CAR stimulation than Tconv cells, and that 28ζ CAR-Tregs make significantly more IL-10 than BBζ Tregs (FIG. 27D). CAR-Tregs also proliferated in response to either CAR or TCR stimulation, except in the case of Δζ CAR-Tregs which, as expected, did not proliferate in response to CAR stimulation (FIG. 28A-28C). It appears that CAR-modified regulatory T cells can be activated and proliferate through their CAR with either CD28 or 4-1BB costimulation while maintaining their Treg identity by their surface phenotype and cytokine profile.

### Example 22. Suppressive Functionality of CAR Tregs

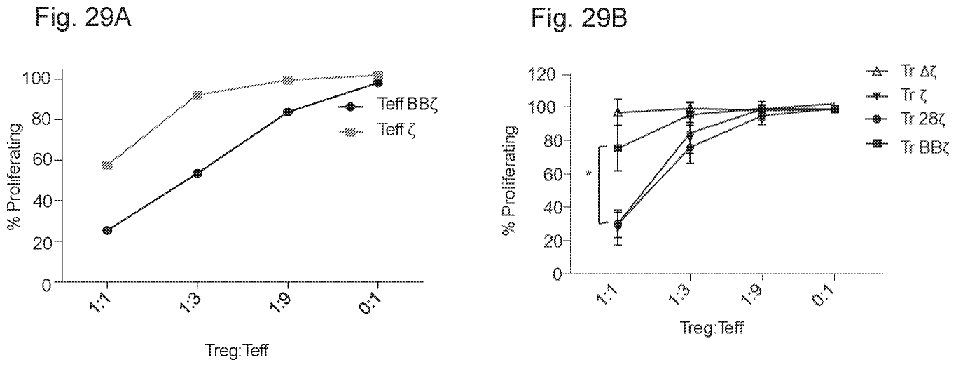

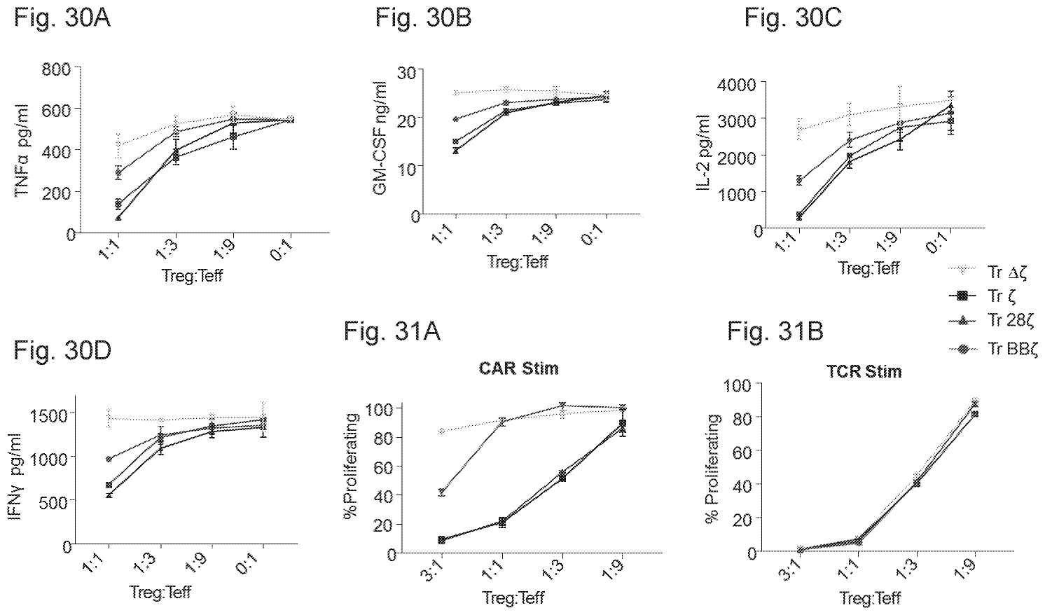

Next, it was determined whether Tregs maintained their suppressive function toward Teff after being modified to express different kinds of CARs. (Note: to avoid confusion, bulk CD4/CD8 T cells to be suppressed by Tregs either in vitro or in vivo will be referred to as Teff cells, while all T cells sorted as CD4⁺ CD25^{low} with the purpose of being directly compared to Tregs will be referred to as Tconv cells). To measure CAR-activated Treg suppression specifically, a system was used whereby Teff cells could be activated without using TCR stimulation. Teff CD4⁺ cells transduced to express a first-generation anti-CD19ζ CAR were chosen as the cells to be suppressed, and irradiated CD19⁺ Nalm6 cells as the antigen presenting cells, in mixed lymphocyte reactions (MLR). A first-generation CAR Teff was specifically chosen because CD19ζ CAR Teff cells were easier to suppress than second-generation CAR-Teff cells (FIG. 29A). MLRs were performed by titrating violet-labeled CAR-Tregs with constant numbers of CFSE-labeled Teff and Nalm6 target cells. Analysis of the CFSE dilution of Teff cells confirmed that all functional CAR-Tregs could inhibit proliferation, except for the negative control Δζ CAR-Tregs. However, 28ζ and ζ CAR-Tregs inhibited the proliferation of Teff cells to a greater degree than BBζ CAR Tregs (FIG. 29B). The levels of inflammatory cytokines in supernatants of the MLRs were also measured, with the expectation that CAR-Treg would inhibit the secretion of these cytokines by CAR-Teff. For these experiments, anti-CD19 CAR 28ζ Teff was used because they secrete the greatest amounts of cytokines. Again, it was observed that BBζ CAR-Tregs were not as efficient as 2K and CAR-Tregs at preventing the secretion of TNFα, GM-CSF, IL-2, and IFNγ from Teff cells (FIGS. 30A-30D, respectively). In summary, CAR-Tregs can suppress Teff cells after CAR antigen-specific activation, but the inclusion of a 4-1BB costimulatory domain in the CAR results in reduced CAR-mediated suppression.

### Example 23. Suppressive Ability of Tregs is Dependent on CAR-Antigen Signaling

Overexpression of CARs in T cells has been shown to result in ligand independent constitutive signaling (Gomes-Silva et al., Cell Rep. 2017; 21(1):17-26). To determine whether the decrease in suppressive capacity seen in the BBζ CAR Tregs was caused by constitutive signaling through the 4-1BB costimulation domain or only by antigen-specific CAR signaling, the MLR experiments were repeated, but with CFSE-labeled, autologous, resting Teff cells with anti-CD3/anti-CD28 beads for pan-T cell activation. Again, it was observed that CARζ and CAR 28ζ Tregs were able to suppress CARL Teff, whereas CAR Δζ could not, and CAR BBζ Tregs suppressed proliferation poorly (FIG. 31A). In contrast, Tregs transduced with any CAR but stimulated through the TCR displayed equal ability to suppress Teff cells (FIG. 31B). It appears that antigen specific signaling from CARs containing 4-1BB signaling domains inhibit Treg suppressive function.

### Example 24. CAR Treg Mechanism of Suppression

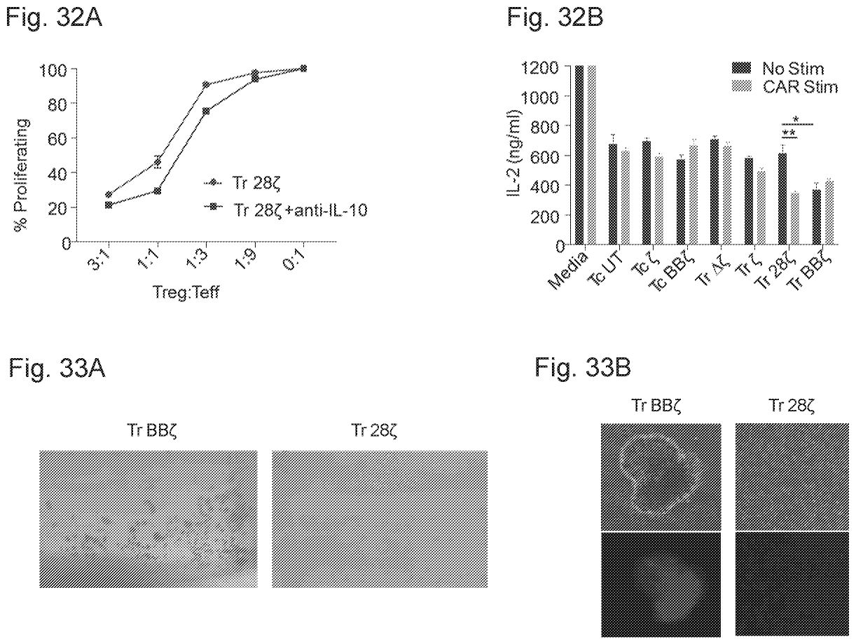

Next, the mechanism of reduced immunosuppression in BBζ CAR-Tregs was explored. Tregs are thought to suppress Teff proliferation through a variety of mechanisms, including the secretion of inhibitory cytokines TGFβ and IL-10, and the consumption of IL-2 (Vignali et al., Front Immunol. 2012; 3:191; Gastegier et al., Front Immunol. 2012; 3:179; Thornton et al., J Immunol. 2004; 172(11):6519-6523). Because it was observed that activated BBζ CAR-Tregs secrete less IL-10 than 28ζ CAR T cells, it was investigated whether IL-10 was contributing to Teff cell suppression by CAR-Tregs. Suppression assays were repeated with IL-10 blocking antibodies; however, no effect of blocking IL-10 on Treg suppression was observed (FIG. 32A). Next, it was determined whether IL-2 consumption upon activation was different among various CAR-Tregs. We measured IL-2 in the media of CAR-Tconv, or CAR-Tregs after 40 hours in culture in the absence or presence of antigen (irradiated CD19⁺ Nalm6 cells). Without stimulation, BBζ CAR-Tregs consumed more IL-2 than Tconv or other CAR-Tregs (FIG. 32B), suggesting that BBζ Tregs are more metabolically active at baseline, which is also consistent with the increased CD69 expression that we found in rested BB CAR-Tregs (FIG. 26A). However, BBζ CAR-Tregs did not significantly increase their consumption of IL-2 after antigen stimulation, which may partly, but not fully explain their reduced immunosuppressive capacity, given that the consumption of IL-2 was similar to activated CD28t CAR-Tregs. In contrast, 28 CAR-Tregs consumed less IL-2 than BBζ CAR-Tregs at rest, but they consumed equal or more IL-2 after antigen stimulation (FIG. 32B). Treg-mediated suppression has also been shown to require cell-cell contact in vitro (Hagness et al., J Immunol. 2012; 188(11):5459-5466). It was observed that BBζ CAR-Tregs formed large cell aggregates, unlike any of the other CAR-Tregs (FIGS. 33A and 33B). Potentially, this aggregation of BBζ CAR-Tregs leads to dysfunctional interactions between CAR-Tregs and Teff cells, thereby affecting BBζ CAR-Treg suppression. In summary, at baseline BBζ CAR-Tregs consume more IL-2, express higher CD69, and aggregate with each other, but none of these findings seem to fully explain how 4-1BB CARs abrogate antigen-mediated immunosuppression.

### Example 25. Cytolytic Activity of CAR Tregs In Vitro

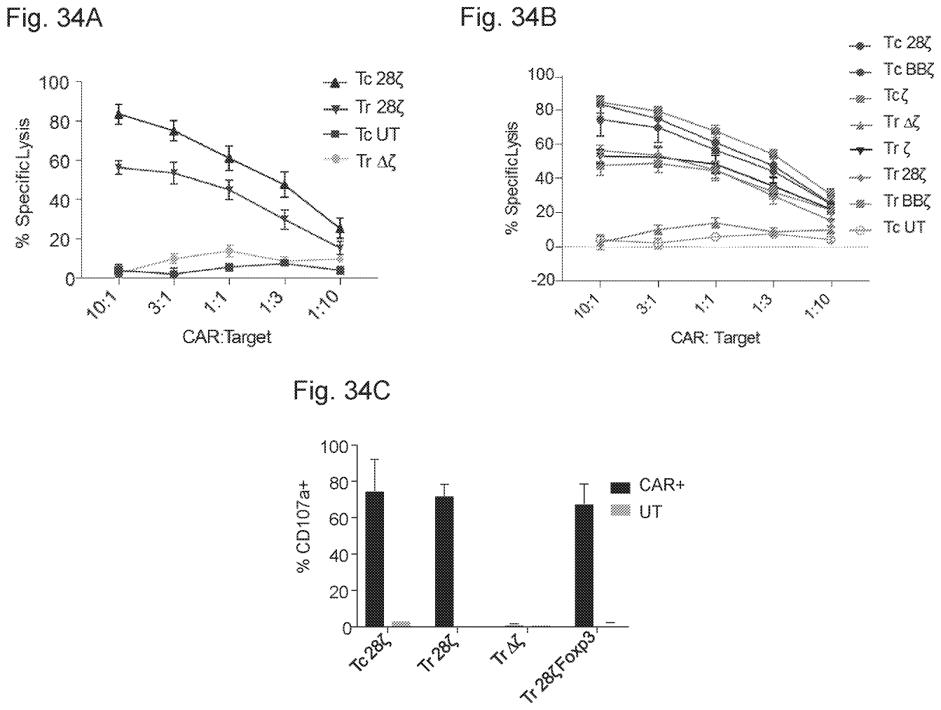

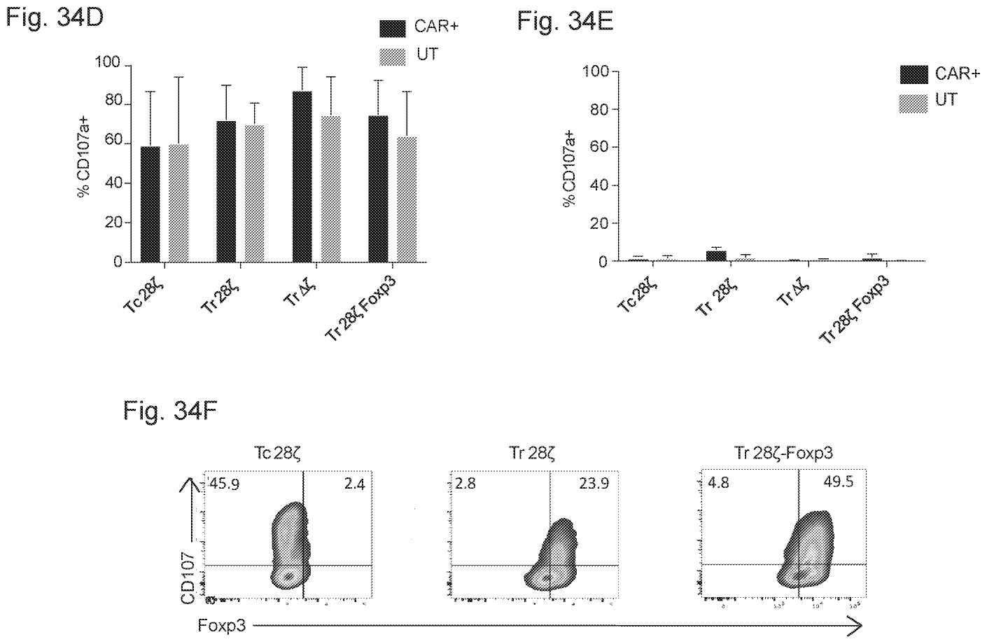

CAR-modified CD4⁺ Tconv cells can kill antigen-expressing targets cells at similar efficiency to the classic cytotoxic CD8⁺ T cell (Yang et al., Sci Transl Med. 2017; 9(417)). In addition to their ability to suppress proliferation of Teff, Tregs can induce apoptosis in Teff through granzyme B mediated cytolysis (Cao et al., Immunity. 2007; 27(4):635-646; Gondek et al., J Immunol. 2005; 174(4):1783-1786). It was determined whether CD19 CAR-Tregs would gain similar cytolytic function and induce apoptosis in cells expressing CD19. Cytotoxicity was measured by titrating CAR-Tconv or CAR-Tregs with CD19⁺ Nalm6 cells. It was observed that all functional CAR-Tregs killed their target cells, irrespective of their costimulatory domain, but with a significantly lower efficiency than CD4⁺ Tconv cells (FIG. 34A and FIG. 34B). To confirm that target specific cytolysis was not due to contamination of Foxp3⁻ Tconv cells in the Treg cultures, a flow-cytometry based degranulation assay was performed, such the degranulation of CAR⁺ cells that did or did not express intracellular Foxp3 could be examined. The Tregs transduced with the CAR-28ζ-Foxp3 construct was also included in these assays. Clear degranulation by both Tconv and Treg CAR T cells when incubated with CD19⁺ Nalm6 cells was observed (FIG. 34C), as well as robust degranulation when incubated with PMA/ionomycin (FIG. 34D) and no degranulation in the absence of CAR or TCR stimulation (FIG. 34E). Notably, by dual staining, it was also confirmed that antigen stimulation resulted in degranulation of Foxp3⁻ 28ζ CAR-Tconv cells and Foxp3⁺ 28 ζ CAR-Treg cells (FIG. 34F). The addition of transgenic Foxp3 did not prevent CAR-Treg degranulation, further establishing that degranulation was not due Foxp3⁻ contaminating cells (FIG. 34F).

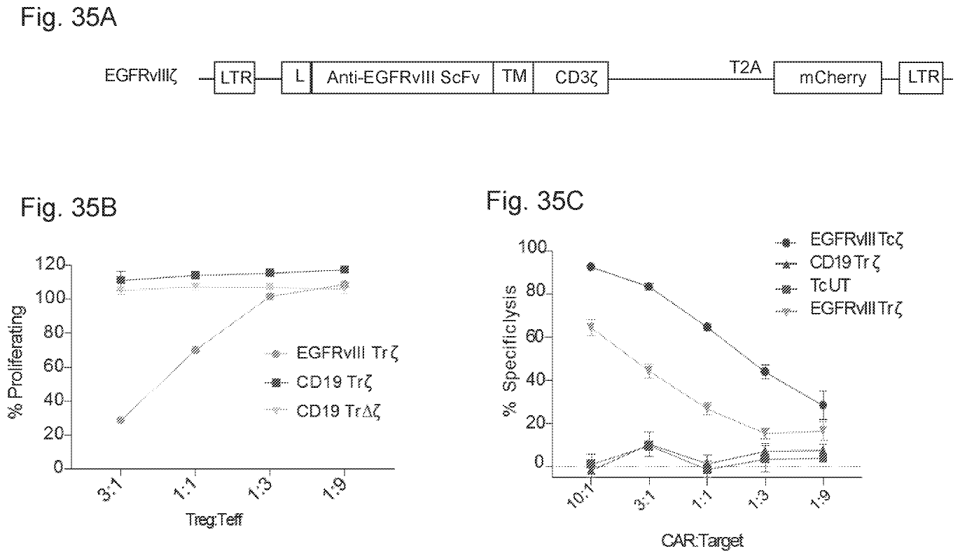

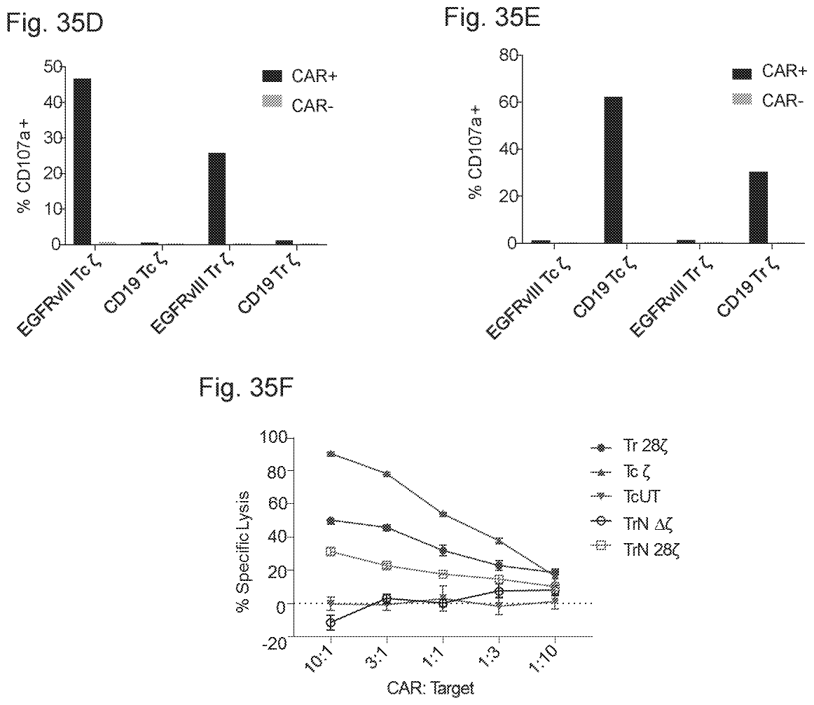

CAR-Tregs directed to HLA-A2 did not have significant cytotoxicity towards target cells in vitro (MacDonald et al., J. Clin. Invest. 2016; 126(4):1413-1424; Boardman et al., Am J Transplant. 2017; 17(4):931-943). It was hypothesized that the cytotoxicity observed in the CAR-Tregs was dependent on the high affinity CD19 scFv binder. To test this hypothesis, a first-generation CAR using an scFv against EGFRvIII was generated (FIG. 35A), which included a lower affinity scFv compared to the CD19 scFv (EC₅₀ of ˜6 ng vs ˜100 ng) (Sommermeyer et al., Leukemia. 2017; 31(10):2191-2199; Johnson et al., Sci Transl Med. 2015; 7(275):275ra222). First, it was confirmed that the first generation EGFRvIII CAR-Tregs can suppress antigen-specific CAR-Teff cells (FIG. 35B). Once again, it was also observed that the CAR-Tregs could kill target cells (FIG. 35C) and degranulate in the presence of target antigen (FIGS. 35D and 35E). Finally, the possibility that whether sorting ‘naïve’ Tregs based on the positive expression of CD45RA would prevent target-specific cytolysis by Tregs was investigated. Though CD45RA⁺ CAR-Tregs displayed less cytolytic activity than bulk Tregs, they still had higher target cell specific lysis when incubated with Nalm6 cells compared to Δζ Tregs and UT Tconv cells (FIG. 35F). Therefore, neither lower affinity antigen nor using naïve cells can prevent CAR-mediated Treg cytotoxicity in vitro.

### Example 26. Cytotoxicity of CAR Tregs is Dependent on Granzyme B

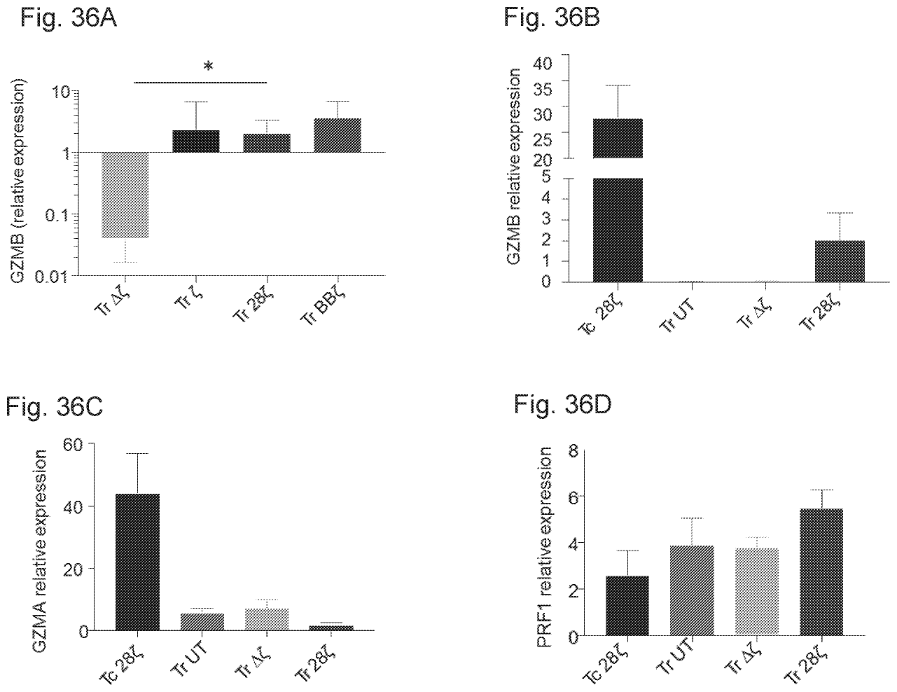

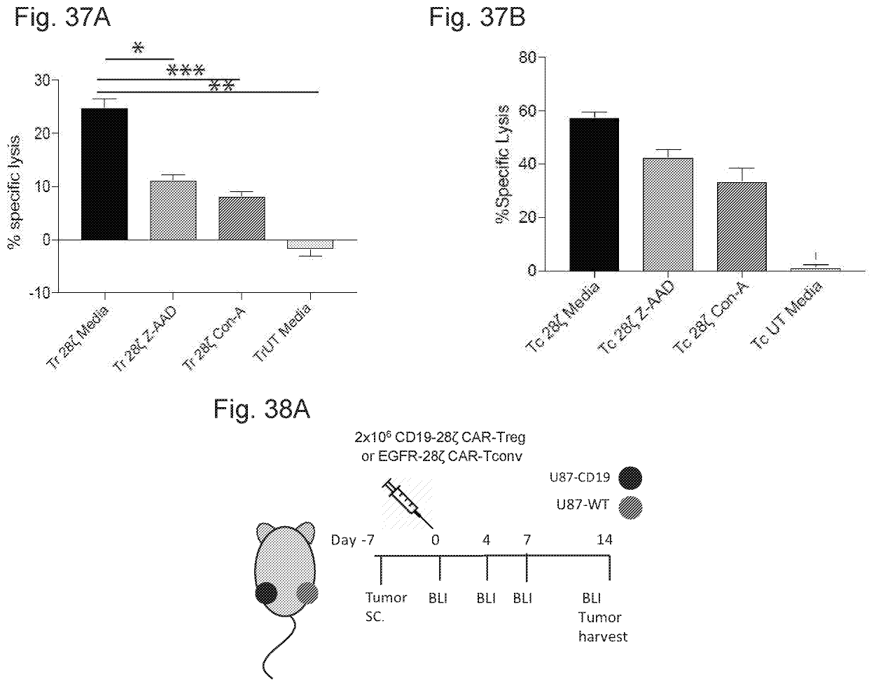

Given the robust antigen-specific degranulation of CAR-Treg cells in response to antigen, perhaps the mechanism of CAR-Treg cytotoxicity was via release of cytotoxic granules. To test this, digital droplet PCR was performed to determine the expression levels of GZMB, GZMA and PRF1 (encoding proteins granzyme B, granzyme A and perforin respectively) in CD19 CAR-Tregs and Tconv cells after a 24-hour stimulation with CD19+ target (Nalm6) cells. It was found that GZMB was specifically upregulated in functional, activated CAR Tregs compared to Δζ Tregs (FIG. 36A), though GZMB expression was not as high as in activated CAR-Tconv cells (FIG. 36B). GZMA was not induced by CAR activation, and PRF1 expression was similar across CAR Tregs and Tconv cells (FIGS. 36C and 36D, respectively). Addition of perforin/granzyme B pathway inhibitors with either Concanamycin A, a perforin inhibitor, or Z-AAD-CMK, a granzyme B specific inhibitor, reduced both Treg (FIG. 37A) and Tconv cytotoxicity (FIG. 37B). In conclusion, CAR-modified Tregs display a low but significant level of target cell-specific lysis in vitro that is at least partly dependent on the granzyme B/perforin pathway.

### Example 27. In Vivo Models of CAR Treg Trafficking, Suppression, and Cytotoxicity

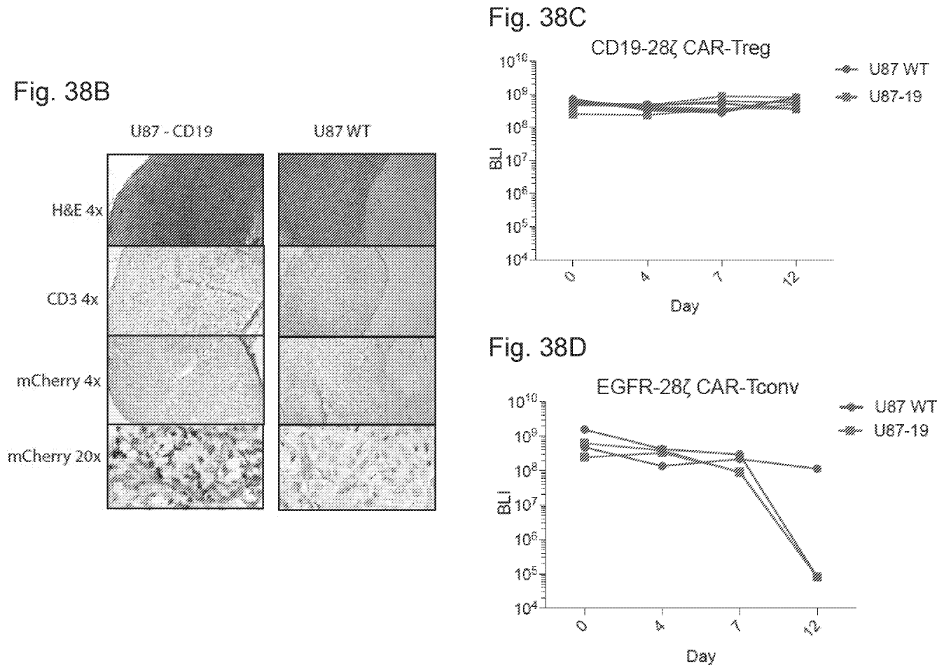

To test whether CAR-Tregs traffic specifically towards their CAR-target in vivo, NSG mice were injected with CD19⁻ or CD19⁺ U87 solid tumor-cell lines subcutaneously (SC) into the left and right flank, respectively. Tumor-cell lines expressed click beetle green and were tracked by bioluminescent imaging (BLI) in the mice. One week later (day 0), we injected the mice intravenously (IV) with either anti-CD19 CAR-28ζ CAR-Treg or anti-EGFR 28ζ CAR Tconv as an internal control. Mice were administered IL-2 intraperiotenally (IP) times a week from day 0 to support the injected Tregs. Tumors were harvested at day 14 and examined histologically for tumor necrosis, and the presence of CAR-T cells (FIG. 38A). It was observed that CD19 28ζCAR-Tregs trafficked specifically to the U87-CD19⁺ expressing tumor, whereas this trafficking was not observed to the U87-CD19⁻ tumor by immunohistochemistry (IHC) for CD3 or mCherry (FIG. 38B). In contrast, EGFR Tconv cells trafficked to both left and right tumors since both U87 based cells-lines expressed EGFR (data not shown). CAR-Tregs did not decrease tumor BLI in U87-CD19⁺ or U87-CD19⁻ (FIG. 38C). In contrast, anti-EGFR CAR Tconv cells decreased luminescence of both U87-CD19⁺ and U87-CD19⁻ tumors⁻. (FIG. 38D). In conclusion. CAR-Tregs traffic to sites of antigen but their cytotoxicity is not potent enough to lyse proliferating tumor cells in vivo.

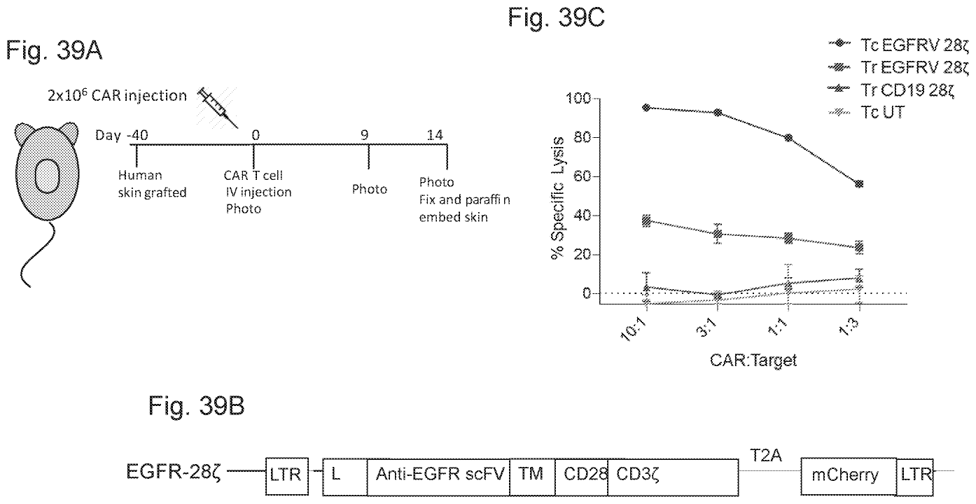

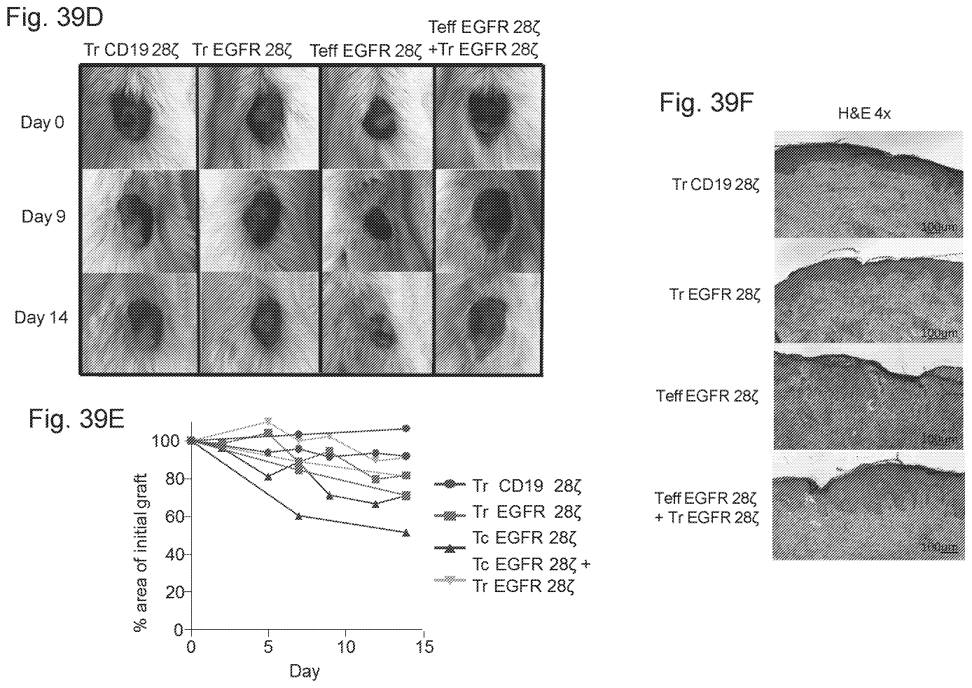

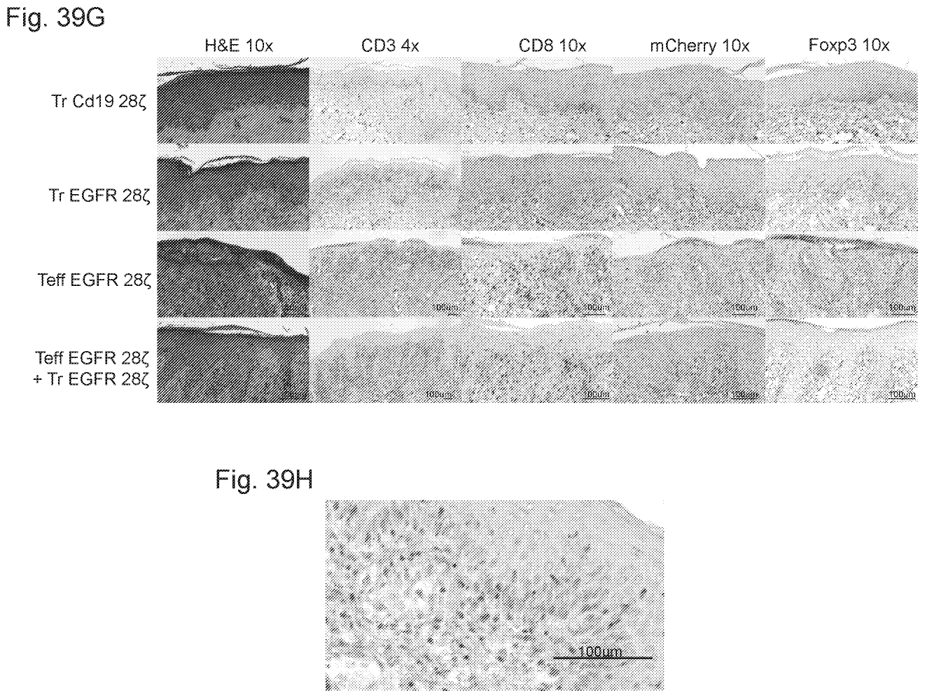

It was hypothesized that if Tregs were to cause low levels of target specific destruction, it would be more relevant and observable in a non-tumor model, where the target cells are not able to proliferate at the rapid rate of tumor cells. A skin xenograft model was therefore used, where NSG mice were grafted with human skin that endogenously expresses EGFR (FIG. 39A). EGFR-28ζ CAR-Tregs and CAR Teff cells were generated (FIG. 39B), and it was confirmed that EGFR 28ζ CAR-Tregs also had low but measurable cytotoxic activity against EGFR+ tumor target cells in vitro (FIG. 39C). After the skin grafts had healed, EGFR 28ζ CAR-Tregs or EGFR 28 ζ CAR-Teff or equal ratios of the two were injected intravenously. CD19 28ζ CAR-Tregs were injected as a negative control. Mice that received Tregs alone were injected with IL-2 as above. Grafts were monitored and photographed over two weeks and the grafts were harvested for histology at day 14 (FIG. 39A). The grafts of the mice that received Teff cells alone had reduced in size and showed clear signs of depigmentation compared to the xenografts of mice that received both CAR-Teff cells and CAR-Tregs (FIG. 39D and FIG. 39E). However, in mice treated with the same number of Teff CARs but in combination with EGFR 28ζ Tregs, the graft had not changed in size and there was no observable skin depigmentation (FIG. 39D). As expected, by H&E (FIG. 39F) and CD3 IHC (FIG. 39G), all the mice that were treated with EGFR CAR T cells—both Tregs and Teff—had clear lymphocyte infiltration into the skin grafts. Graft rejection was obvious by histopathology in the Teff treated graft, with dense lymphocyte infiltration, spongiosis, and exocytosis of the epidermal layer. In some areas, the epithelium had been destroyed and there were signs of epithelial apoptosis, dyskeratosis and keratinolysis (FIG. 39F and FIG. 39G). However, T cell infiltration alone did not dictate graft rejection in this model. It was noted that Tregs remained closer to the dermal/epidermal junction, unlike the Teff cells that infiltrated the epidermal layer (FIG. 39F). The signs of epithelial barrier destruction that were seen in the CAR-Teff alone group were reduced when EGFR CAR-Tregs were administered with Teff cells, indicating that in vivo the CAR-Tregs could suppress Teff cell tissue destruction (FIG. 39F and FIG. 39G). Furthermore, the Treg suppressive function was dominant over Teff rejection. Additionally, CD19-CAR Tregs could not be detected in the skin graft. Further, IHC for CD8, Foxp3 and mCherry antibodies confirmed that the EGFR CAR T cells infiltrating the grafts were indeed mCherry⁺ CAR T cells (FIG. 39G). Foxp3 IHC staining revealed that the mice treated with EGFR CAR-Tregs alone or in combination with Teff cells had nuclear Foxp3 staining of some of the cells whereas the Teff-only treated grafts had only rare Foxp3⁺ cells in the graft (FIG. 39G and FIG. 39H).

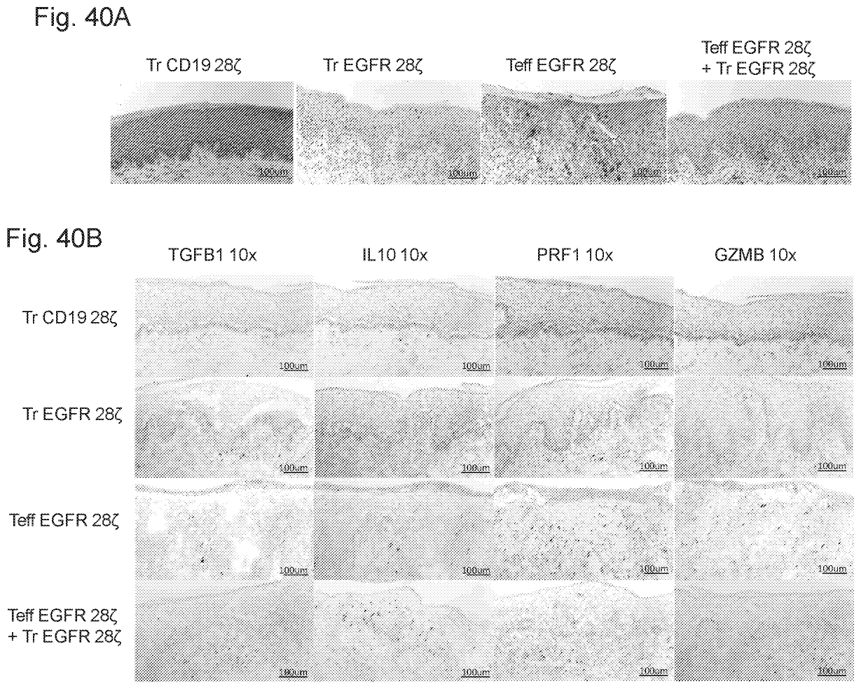

Finally, although there were no signs of overt graft rejection by photographs or histology analysis of EGFR 28ζ CAR Tregs, TUNEL staining showed minimal but observable keratinocyte death, which was also evident in the CAR Treg/CAR Teff combination (FIG. 40A). There was no discernible keratinocyte death or TUNEL staining in the grafts recovered from CD19 CAR-Treg treated mice. In conclusion, CAR Tregs can mediate low but measurable cytotoxicity against tissues expressing the target antigen. To determine CAR T cell functions in the grafts, RNAscope was used to detect IL10 and TGFB1 (encoding immunosuppressive cytokines IL-10 and TGFβ respectively) as well as GZMB and PRF1. It was observed that RNA expression of IL10 and TGFB1 in the grafts with Treg alone, with greatly increased in grafts from mice administered the combination of EGFR CAR-Tregs and CAR-Teff cells (FIG. 40B). In contrast, GZMB and PRF1 were expressed at high levels in Teff alone treated grafts, but at a lower level in the Teff with Treg-treated grafts (FIG. 40B). Significant levels of PIM or GZMB expression in EGFR CAR-Treg alone or CD19 CAR-Treg treated mice were not detected. Therefore, CAR-Tregs bearing CD28ζ signaling domains can traffic to target tissues in vitro and exert functional immunosuppression, despite low levels of target-directed cytotoxicity.

It was demonstrated in this experiment that the 28ζ CAR-T cells trafficked to the site of antigen expression, and that the immunosuppressive activity of the 28 ζ CAR-Tregs was dominant over the cytotoxic activity of the 28ζ CAR-Teff in antigen-expressing tissue (FIG. 39D; far right column labeled “Teff EGFR 28ζ+Tr EGFR 28ζ”). These data show that the CAR-Tregs are capable of mediating a zone of immunosuppression in a tissue (e.g., skin), even when the tissue is targeted by large numbers of cytotoxic T cells that would quickly destroy it.

The invention is further described in the following numbered paragraphs.
1. An engineered Treg cell comprising:
   a. a chimeric antigen receptor; and/or
   b. a nucleic acid encoding said chimeric antigen receptor.
2. The cell of paragraph 1, wherein the chimeric antigen receptor comprises an extracellular domain that specifically binds to a first target molecule expressed on the surface of a first target cell or tissue.
3. The cell of paragraph 2, wherein the first target cell is a cell in a tissue affected by an autoimmune condition and/or allograft rejection.
4. The cell of any of paragraphs 1-3, wherein the cell further comprises
   a. a second chimeric antigen receptor; and/or
   b. a nucleic acid encoding said second chimeric antigen receptor,
   wherein the second chimeric antigen receptor comprises an extracellular domain that specifically binds a different target molecule than the first chimeric antigen receptor.
5. The cell of paragraph 4, wherein the second chimeric antigen receptor comprises an extracellular domain that specifically binds to a second target molecule expressed on the surface of a second target cell.
6. The cell of paragraph 5, wherein the second target cell is an immune system cell contributing to an autoimmune condition and/or allograft rejection.
7. The cell of any of paragraphs 5-6, wherein the second target cell is a Treg cell.
8. The cell of any of paragraphs 5-7, wherein the second target molecule is selected from the group consisting of:
   CTLA4; CD25; CD27; PDL1; GARP; TGFbeta; and LAP.
9. The cell of any of paragraphs 5-6, wherein the second target cell is a myeloid derived suppressor cell (MDSC).
10. The cell of any of paragraphs 5-6 and 9, wherein the second target molecule is selected from the group consisting of:
   CD32; CD33, and CD11c.
11. The cell of any of paragraphs 1-10, wherein a chimeric antigen receptor comprises:
   i. an extracellular target-binding domain;
   ii. a hinge/transmembrane domain; and
   iii. an intracellular co-stimulation domain.
12. The cell of any of paragraphs 1-10, wherein a chimeric antigen receptor comprises:
   i. an extracellular target-binding domain;
   ii. a hinge/transmembrane domain;
   iii. an intracellular co-stimulation domain; and
   iv. a CD3z chain.
13. The cell of any of paragraphs 1-12, wherein the chimeric antigen receptor further comprises an N-terminal leader sequence.
14. The cell of any of paragraphs 1-13, wherein a Treg cell is a CD8− CD4+ CD25+ CD127+ cell.
15. The cell of any of paragraphs 1-13, wherein a Treg cell is a CD8− CD4dim CD25 hi and CD127 low cell.
16. The cell of any of paragraphs 1-15, wherein the Treg cell is a T cell expressing one or more markers selected from the group consisting of:
   CTLA4; PDL1; LAP; GARP; CD25; and CD27.
17. The cell of any of paragraphs 1-16, wherein the leader sequence is a CD8 leader sequence.
18. The cell of any of paragraphs 1-17, wherein the hinge/transmembrane domain is a CD8 hinge/transmembrane domain.
19. The cell of any of paragraphs 1-18, wherein the intracellular co-stimulation domain is a 4-1BB intracellular co-stimulation domain.
20. The cell of any of paragraphs 1-18, wherein the intracellular co-stimulation domain is a CD28 intracellular co-stimulation domain.
21. The cell of any of paragraphs 1-20, wherein the target-binding domain is an antibody reagent.
22. The cell of any of paragraphs 1-20, wherein the target-binding domain is an scFv.
23. The cell of any of paragraphs 1-22, wherein the first target molecule is CD19.
24. The cell of any of paragraphs 1-23, wherein the cell is a mammalian cell.
25. The cell of any of paragraphs 1-24, wherein the cell is a human cell.
26. The cell of any of paragraphs 1-24, wherein the cell is a murine cell.
27. The cell of any of paragraphs 1-26, wherein the cell is autologous to a subject.
28. The cell of any of paragraphs 1-26, wherein the cell is allogeneic to a subject.
29. The cell of any of paragraphs 1-28, wherein the cell is further engineered to reduce expression of an endogenous T cell receptor and/or an endogenous MHC complex.
30. The cell of any of paragraphs 1-20, wherein the cell further comprises:
   a. exogenous FoxP3; CTLA4; PDL1; and/or TGFbeta polypeptides; and/or
   b. an exogenous nucleic acid encoding FoxP3; CTLA4; PDL1; and/or TGFbeta polypeptides.
31. The cell of any of paragraphs 1-30, wherein the cell is further engineered to reduce T cell cytotoxicity function.
32. The cell of paragraph 31, wherein the cell is further engineered to reduce perforin, granzyme, and/or Fas-ligand gene expression.
33. A method of treating or preventing an autoimmune condition or allograft rejection in a subject in need thereof, the method comprising administering an engineered Treg cell of any of paragraphs 1-33 to the subject.
34. A method of treating or preventing an autoimmune condition or allograft rejection in a subject in need thereof, the method comprising:
   a. engineering a Treg cell to express at least a first chimeric antigen receptor;
   b. administering the engineered Treg cell to the subject.
35. A method of treating or preventing an autoimmune condition or allograft rejection in a subject in need thereof, the method comprising:
   a. engineering a Treg cell to express at least a first chimeric antigen receptor;
   b. stimulating the Treg cell resulting from step a); and
   c. administering the stimulated Treg cell to the subject.
36. The method of paragraph 35, wherein the simulating comprises contacting the cell with CD3 and/or CD28.
37. The method of any of paragraphs 33-36, wherein the autoimmune condition is diabetes, neurologic disease, or graft-vs-host disease.
38. The method of any of paragraphs 33-37, wherein a therapeutically effective amount of the cells are administered to the subject.
39. An engineered regulatory T cell (Treg) comprising reduced T cell cytotoxicity function.
40. The Treg of paragraph 39, wherein perforin, granzyme, and/or Fas-ligand gene expression is reduced.
41. The Treg of paragraph 39 or 40, wherein the Treg comprises a chimeric antigen receptor (CAR) and/or a nucleic acid capable of encoding the CAR.
42. The Treg of paragraph 41, wherein the CAR comprises (i) an extracellular domain comprising an antigen-binding sequence, (ii) a transmembrane domain, and (iii) a T cell intracellular signaling domain.
43. The Treg of paragraph 41 or 42, wherein the CAR further comprises (iv) one or more co-stimulatory domains.
44. The Treg of any one of paragraphs 41-43, wherein the CAR further comprises an N-terminal leader sequence.
45. The Treg of any one of paragraphs 41-44, wherein the leader sequence is a CD8 leader sequence.
46. The Treg of any one of paragraphs 41-45, wherein the antigen-binding sequence is an antibody reagent.
47. The Treg of any one of paragraphs 41-46, wherein the antigen-binding sequence is an scFv.
48. The Treg of any one of paragraphs 41-47, wherein the CAR further comprises a hinge domain selected from the group consisting of the hinge domains of CD8, CD4, CD28 and CD7.
49. The Treg of paragraph 48, wherein the hinge domain is a CD8 hinge domain.
50. The Treg of any one of paragraphs 41-49, wherein the transmembrane domain is selected from the group consisting of the transmembrane domains of the alpha, beta, and zeta chains of the T-cell receptor, CD3ε, CD3ζ, CD4, CD5, CD8, CD9, CD16, CD22, CD27, CD28, CD33, CD37, CD45, CD64, CD80, CD86, CD134 (OX40), CD137 (4-1BB), CD152 (CTLA4), CD154, and PD-1.
51. The Treg of paragraph 50, wherein the transmembrane domain is a CD8 transmembrane domain.
52. The Treg of any one of paragraphs 41-51, wherein the T cell intracellular signaling domain is selected from the group consisting of the intracellular signaling domains of TCRζ, FcRγ, FcRβ, CD3γ, CD3δ, CD3ε, CD3ζ, CD22, CD79a, CD79b, and CD66d.
53. The Treg of paragraph 52, wherein the T cell intracellular signaling domain is a CD3 intracellular signaling domain.
54. The Treg of any one of paragraphs 41-53, wherein the co-stimulatory domain is selected from the group consisting of the co-stimulatory domains of CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70.
55. The Treg of paragraph 54, wherein the co-stimulatory domain is a 4-1BB co-stimulatory domain.
56. The Treg of paragraph 54, wherein the co-stimulatory domain is a CD28 co-stimulatory domain.
57. The Treg of any one of paragraphs 41-56, wherein the antigen-binding sequence is specific to an antigen expressed by a cell affected by a disease or disorder.
58. The Treg of paragraph 57, wherein the disease or disorder is an autoimmune disease and/or an allograft rejection.
59. The Treg of any one of paragraphs 41-58, wherein the Treg further comprises a second CAR, and/or a nucleic acid encoding a second CAR, comprising (i) a second extracellular domain comprising a second antigen-binding sequence, (ii) a second transmembrane domain, and (iii) a second T cell intracellular signaling domain, and wherein the second antigen-binding sequence is specific to a second antigen different from the first antigen-binding sequence.
60. The Treg of paragraph 59, wherein the CAR further comprises (iv) one or more co-stimulatory domains.
61. The Treg of paragraph 59-60, wherein the second antigen is expressed by an immune cell contributing to an autoimmune disease.
62. The Treg of paragraph 61, wherein the immune cell is a Treg.
63. The Treg of paragraph 62, wherein the second antigen is selected from the group consisting of CTLA4, CD25, CD27, PD-L1, GARP, TGFβ, and LAP.
64. The Treg of paragraph 61, wherein the immune cell is a myeloid derived suppressor cell (MDSC).
65. The Treg of any one of paragraphs 63, wherein the second antigen is selected from the group consisting of CD32, CD33, and CD11c.
66. The Treg of any one of paragraphs 39-65, wherein the Treg is a mammalian cell.
67. The Treg of any one of paragraphs 39-66, wherein the Treg is a human cell.
68. The Treg of any one of paragraphs 39-66, wherein the Treg is a murine cell.
69. The Treg of any one of paragraphs 39-68, wherein the Treg is autologous to a subject.
70. The Treg of any one of paragraphs 39-68, wherein the Treg is allogeneic to a subject.
71. The Treg of any one of paragraphs 39-70, wherein the Treg is further engineered to reduce expression of an endogenous T cell receptor and/or an endogenous MHC complex.
72. The Treg of any one of paragraphs 39-71, wherein the Treg further comprises
   a) exogenous Foxp3, CTLA4, PD-L1, and/or TGFβ polypeptides; and/or
   b) an exogenous nucleic acid encoding Foxp3, CTLA4, PD-L1, and/or TGFβ polypeptides.
73. The Treg of any one of paragraphs 39-72, wherein the Treg is a CD8−, CD4+, CD25+, and CD127+ cell.
74. The Treg of any one of paragraphs 39-72, wherein the Treg is a CD8−, CD4dim, CD25hi, and CD127low cell.
75. The Treg of any one of paragraphs 39-74, wherein the Treg expresses one or more markers selected from the group consisting of CTLA4, PD-L1, LAP, GARP, CD25, and CD27.
76. A pharmaceutical composition comprising the Treg of any one of paragraphs 39-75 and a pharmaceutically acceptable carrier.
77. A method of treating or preventing an autoimmune disease and/or an allograft rejection in a subject in need thereof, the method comprising administering the Treg of any one of paragraphs 39-75 or the pharmaceutical composition of paragraph 76 to the subject.
78. A method of treating or preventing an autoimmune disease and/or an allograft rejection in a subject in need thereof, the method comprising:
   (a) engineering a Treg to reduce T cell cytotoxicity functions, and
   (b) administering the engineered Treg to the subject.
79. A method of treating or preventing an autoimmune disease and/or an allograft rejection in a subject in need thereof, the method comprising:
   (a) engineering a Treg to reduce T cell cytotoxicity functions, wherein the Treg comprises a CAR and/or a nucleic acid capable of encoding said CAR, and
   (b) administering the engineered Treg to the subject.
80. A method of treating or preventing an autoimmune disease and/or an allograft rejection in a subject in need thereof, the method comprising:
   (a) engineering a Treg to reduce T cell cytotoxicity functions, wherein the Treg comprises a CAR and/or a nucleic acid capable of encoding said CAR,
   (b) stimulating the Treg of step (a), and
   (c) administering the stimulated Treg to the subject.
81. The method of paragraph 80, wherein the stimulating comprises contacting the cell with CD3 and/or CD28.
82. The method of any one of paragraphs 79-81, wherein the Treg is engineered to reduce perforin, granzyme, and/or Fas-ligand gene expression.
83. The method of any one of paragraphs 79-82, wherein the CAR is specific to an antigen expressed by a cell affected by the autoimmune disease and/or allograft rejection.
84. The method of any one of paragraphs 77-83, wherein the autoimmune disease is diabetes, neurologic disease, or graft versus host disease.
85. The method of any one of paragraphs 77-84, wherein a therapeutically effective amount of Tregs is administered to a subject.
86. A method of providing immunosuppression in a solid tissue in a subject, the method comprising administering to the subject an engineered regulatory T (Treg) cell comprising a chimeric antigen receptor (CAR), wherein the CAR comprises:
   (i) an extracellular domain comprising an antigen-binding domain,
   (ii) a transmembrane domain,
   (iii) a signaling domain, and
   (iv) a co-stimulatory domain.
87. The method of paragraph 86, wherein the antigen-binding domain binds to an antigen expressed on the solid tissue.
88. The method of paragraph 87, wherein the antigen is expressed on a skin cell.
89. The method of paragraph 88, wherein the antigen is epidermal growth factor receptor (EGFR).
90. The method of any one of paragraphs 86-89, wherein the signaling domain is a CD3ζ signaling domain.
91. The method of any one of paragraphs 86-90, wherein the co-stimulatory domain is a CD28 co-stimulatory domain.
92. The method of any one of paragraphs 86-91, wherein the solid tissue is skin

What is claimed is:

1. A method of providing immunosuppression in a solid tissue in a subject, the method comprising administering to the subject an engineered regulatory T (Treg) cell comprising a chimeric antigen receptor (CAR), wherein the CAR comprises:
(i) an extracellular domain comprising an antigen-binding domain,
(ii) a transmembrane domain,
(iii) a signaling domain, and
(iv) a co-stimulatory domain.

2. The method of claim 1, wherein the antigen-binding domain binds to an antigen expressed on the solid tissue.

3. The method of claim 2, wherein the antigen is expressed on a skin cell.

4. The method of claim 3, wherein the antigen is epidermal growth factor receptor (EGFR).

5. The method of claim 1, wherein the signaling domain is a CD3ζ signaling domain.

6. The method of claim 1, wherein the co-stimulatory domain is a CD28 co-stimulatory domain.

7. The method of claim 1, wherein the solid tissue is skin.

8. An engineered regulatory T (Treg) cell comprising:
a. a chimeric antigen receptor; and/or
b. a nucleic acid encoding said chimeric antigen receptor.

9. The cell of claim 8, wherein the chimeric antigen receptor comprises an extracellular domain that specifically binds to a first target molecule expressed on the surface of a first target cell or tissue.

10. The cell of claim 9, wherein the first target cell is a cell in a tissue affected by an autoimmune condition and/or allograft rejection.

11. The cell of claim 8, wherein the cell further comprises
a. a second chimeric antigen receptor; and/or
b. a nucleic acid encoding said second chimeric antigen receptor,
wherein the second chimeric antigen receptor comprises an extracellular domain that specifically binds a different target molecule than the first chimeric antigen receptor.

12. The cell of claim 11, wherein the second chimeric antigen receptor comprises an extracellular domain that specifically binds to a second target molecule expressed on the surface of a second target cell.

13. The cell of claim 12, wherein the second target cell is an immune system cell contributing to an autoimmune condition and/or allograft rejection.

14. The cell of claim 12, wherein the second target cell is a Treg cell.

15. The cell of claim 14, wherein the second target molecule is selected from the group consisting of:
CTLA4; CD25; CD27; PDL1; GARP; TGFbeta; and LAP.

16. The cell of claim 12, wherein the second target cell is a myeloid derived suppressor cell (MDSC).

17. The cell of claim 16, wherein the second target molecule is selected from the group consisting of:
CD32; CD33, and CD11c.

18. The cell of claim 8, wherein a chimeric antigen receptor comprises:
i. an extracellular target-binding domain;
ii. a hinge/transmembrane domain; and
iii. an intracellular co-stimulation domain.

19. The cell of claim 18, wherein a chimeric antigen receptor comprises:
i. an extracellular target-binding domain;
ii. a hinge/transmembrane domain;
iii. an intracellular co-stimulation domain; and
iv. a CD3 z chain.

20. The cell of claim 18, wherein the chimeric antigen receptor further comprises an N-terminal leader sequence.

21. The cell of claim 8, wherein the Treg cell is a CD8− CD4+ CD25+ CD127+ cell.

22. The cell of claim 8, wherein the Treg cell is a CD8− CD4dim CD25 hi and CD127 low cell.

23. The cell of claim 8, wherein the Treg cell is a T cell expressing one or more markers selected from the group consisting of:
CTLA4; PDL1; LAP; GARP; CD25; and CD27.

24. The cell of claim 20, wherein the leader sequence is a CD8 leader sequence.

25. The cell of claim 18, wherein the hinge/transmembrane domain is a CD8 hinge/transmembrane domain.

26. The cell of claim 18, wherein the intracellular co-stimulation domain is a 4-1BB intracellular co-stimulation domain.

27. The cell of claim 18, wherein the intracellular co-stimulation domain is a CD28 intracellular co-stimulation domain.

28. The cell of claim 18, wherein the target-binding domain is an antibody reagent.

29. The cell of claim 18, wherein the target-binding domain is an scFv.

30. The cell of claim 9, wherein the first target molecule is CD19.

31. The cell of claim 8, wherein the cell is a mammalian cell.

32. The cell of claim 8, wherein the cell is a human cell.

33. The cell of claim 8, wherein the cell is a murine cell.

34. The cell of claim 8, wherein the cell is autologous to a subject.

35. The cell of claim 8, wherein the cell is allogeneic to a subject.

36. The cell of claim 8, wherein the cell is further engineered to reduce expression of an endogenous T cell receptor and/or an endogenous MHC complex.

37. The cell of claim 8, wherein the cell further comprises:
a. exogenous FoxP3; CTLA4; PDL1; and/or TGFbeta polypeptides; and/or
b. an exogenous nucleic acid encoding FoxP3; CTLA4; PDL1; and/or TGFbeta polypeptides.

38. The cell of claim 8, wherein the cell is further engineered to reduce T cell cytotoxicity function.

39. The cell of claim 38, wherein the cell is further engineered to reduce perforin, granzyme, and/or Fas-ligand gene expression.

40. A method of treating or preventing an autoimmune condition or allograft rejection in a subject in need thereof, the method comprising administering an engineered Treg cell of claim 1 to the subject.

41. A method of treating or preventing an autoimmune condition or allograft rejection in a subject in need thereof, the method comprising:
a. engineering a Treg cell to express at least a first chimeric antigen receptor;
b. administering the engineered Treg cell to the subject.

42. A method of treating or preventing an autoimmune condition or allograft rejection in a subject in need thereof, the method comprising:
a. engineering a Treg cell to express at least a first chimeric antigen receptor;
b. stimulating the Treg cell resulting from step a); and
c. administering the stimulated Treg cell to the subject.

43. The method of claim 42, wherein the simulating comprises contacting the cell with CD3 and/or CD28.

44. The method of claim 40, wherein the autoimmune condition is diabetes, neurologic disease, or graft-vs-host disease.

45. The method of claim 40, wherein a therapeutically effective amount of the cells are administered to the subject.

46. The method of claim 41, wherein the autoimmune condition is diabetes, neurologic disease, or graft-vs-host disease.

47. The method of claim 41, wherein a therapeutically effective amount of the cells are administered to the subject.

48. The method of claim 42, wherein the autoimmune condition is diabetes, neurologic disease, or graft-vs-host disease.

49. The method of claim 42, wherein a therapeutically effective amount of the cells are administered to the subject.

50. An engineered regulatory T cell (Treg) comprising reduced T cell cytotoxicity function.

51. The Treg of claim 50, wherein perforin, granzyme, and/or Fas-ligand gene expression is reduced.

52. The Treg of claim 51, wherein the Treg comprises a chimeric antigen receptor (CAR) and/or a nucleic acid capable of encoding the CAR.

53. The Treg of claim 52, wherein the CAR comprises (i) an extracellular domain comprising an antigen-binding sequence, (ii) a transmembrane domain, and (iii) a T cell intracellular signaling domain.

54. The Treg of claim 53, wherein the CAR further comprises (iv) one or more co-stimulatory domains.

55. The Treg of claim 53, wherein the CAR further comprises an N-terminal leader sequence.

56. The Treg of claim 55, wherein the leader sequence is a CD8 leader sequence.

57. The Treg of claim 56, wherein the antigen-binding sequence is an antibody reagent.

58. The Treg of claim 57, wherein the antigen-binding sequence is an scFv.

59. The Treg of claim 53, wherein the CAR further comprises a hinge domain selected from the group consisting of the hinge domains of CD8, CD4, CD28 and CD7.

60. The Treg of claim 59, wherein the hinge domain is a CD8 hinge domain.

61. The Treg of claim 53, wherein the transmembrane domain is selected from the group consisting of the transmembrane domains of the alpha, beta, and zeta chains of the T-cell receptor, CD3ε, CD3ζ, CD4, CD5, CD8, CD9, CD16, CD22, CD27, CD28, CD33, CD37, CD45, CD64, CD80, CD86, CD134 (OX40), CD137 (4-1BB), CD152 (CTLA4), CD154, and PD-1.

62. The Treg of claim 61, wherein the transmembrane domain is a CD8 transmembrane domain.

63. The Treg of claim 53, wherein the T cell intracellular signaling domain is selected from the group consisting of the intracellular signaling domains of TCR, FcRγ, FcRβ, CD3γ, CD3δ, CD3ε, CD3ζ; CD22, CD79a, CD79b, and CD66d.

64. The Treg of claim 63, wherein the T cell intracellular signaling domain is a CD3 intracellular signaling domain.

65. The Treg of claim 54, wherein the co-stimulatory domain is selected from the group consisting of the co-stimulatory domains of CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70.

66. The Treg of claim 65, wherein the co-stimulatory domain is a 4-1BB co-stimulatory domain.

67. The Treg of claim 65, wherein the co-stimulatory domain is a CD28 co-stimulatory domain.

68. The Treg of claim 53, wherein the antigen-binding sequence is specific to an antigen expressed by a cell affected by a disease or disorder.

69. The Treg of claim 68, wherein the disease or disorder is an autoimmune disease and/or an allograft rejection.

70. The Treg of claim 53, wherein the Treg further comprises a second CAR, and/or a nucleic acid encoding a second CAR, comprising (i) a second extracellular domain comprising a second antigen-binding sequence, (ii) a second transmembrane domain, and (iii) a second T cell intracellular signaling domain, and
wherein the second antigen-binding sequence is specific to a second antigen different from the first antigen-binding sequence.

71. The Treg of claim 70, wherein the second CAR further comprises (iv) one or more co-stimulatory domains.

72. The Treg of claim 70, wherein the second antigen is expressed by an immune cell contributing to an autoimmune disease.

73. The Treg of claim 72, wherein the immune cell is a Treg.

74. The Treg of claim 72, wherein the second antigen is selected from the group consisting of CTLA4, CD25, CD27, PD-L1, GARP, TGFβ, and LAP.

75. The Treg of claim 72, wherein the immune cell is a myeloid derived suppressor cell (MDSC).

76. The Treg of claim 75, wherein the second antigen is selected from the group consisting of CD32, CD33, and CD11c.

77. The Treg of claim 50, wherein the Treg is a mammalian cell.

78. The Treg of claim 77, wherein the Treg is a human cell.

79. The Treg of claim 77, wherein the Treg is a murine cell.

80. The Treg of claim 50, wherein the Treg is autologous to a subject.

81. The Treg of claim 50, wherein the Treg is allogeneic to a subject.

82. The Treg of claim 50, wherein the Treg is further engineered to reduce expression of an endogenous T cell receptor and/or an endogenous MHC complex.

83. The Treg of claim 50, wherein the Treg further comprises
a) exogenous Foxp3, CTLA4, PD-L1, and/or TGFβ polypeptides; and/or
b) an exogenous nucleic acid encoding Foxp3, CTLA4, PD-L1, and/or TGFβ polypeptides.

84. The Treg of claim 50, wherein the Treg is a CD8−, CD4+, CD25+, and CD127+ cell.

85. The Treg of claim 50, wherein the Treg is a CD8−, CD4dim, CD25hi, and CD127low cell.

86. The Treg of claim 50, wherein the Treg expresses one or more markers selected from the group consisting of CTLA4, PD-L1, LAP, GARP, CD25, and CD27.

87. A pharmaceutical composition comprising the Treg of claim 50 and a pharmaceutically acceptable carrier.

88. A method of treating or preventing an autoimmune disease and/or an allograft rejection in a subject in need thereof, the method comprising administering the Treg of claim 50 to the subject.

89. A method of treating or preventing an autoimmune disease and/or an allograft rejection in a subject in need thereof, the method comprising:
(a) engineering a Treg to reduce T cell cytotoxicity functions, and
(b) administering the engineered Treg to the subject.

90. A method of treating or preventing an autoimmune disease and/or an allograft rejection in a subject in need thereof, the method comprising:
(a) engineering a Treg to reduce T cell cytotoxicity functions, wherein the Treg comprises a CAR and/or a nucleic acid capable of encoding said CAR, and
(b) administering the engineered Treg to the subject.

91. A method of treating or preventing an autoimmune disease and/or an allograft rejection in a subject in need thereof, the method comprising:
(a) engineering a Treg to reduce T cell cytotoxicity functions, wherein the Treg comprises a CAR and/or a nucleic acid capable of encoding said CAR,
(b) stimulating the Treg of step (a), and
(c) administering the stimulated Treg to the subject.

92. The method of claim 91, wherein the stimulating comprises contacting the cell with CD3 and/or CD28.

93. The method of claim 89, wherein the Treg is engineered to reduce perforin, granzyme, and/or Fas-ligand gene expression.

94. The method of claim 89, wherein the CAR is specific to an antigen expressed by a cell affected by the autoimmune disease and/or allograft rejection.

95. The method of claim 94, wherein the autoimmune disease is diabetes, neurologic disease, or graft versus host disease.

96. The method of claim 89, wherein a therapeutically effective amount of Tregs is administered to a subject.

97. The method of claim 90, wherein the Treg is engineered to reduce perforin, granzyme, and/or Fas-ligand gene expression.

98. The method of claim 90, wherein the CAR is specific to an antigen expressed by a cell affected by the autoimmune disease and/or allograft rejection.

99. The method of claim 98, wherein the autoimmune disease is diabetes, neurologic disease, or graft versus host disease.

100. The method of claim 90, wherein a therapeutically effective amount of Tregs is administered to a subject.

101. The method of claim 91, wherein the Treg is engineered to reduce perforin, granzyme, and/or Fas-ligand gene expression.

102. The method of claim 91, wherein the CAR is specific to an antigen expressed by a cell affected by the autoimmune disease and/or allograft rejection.

103. The method of claim 102, wherein the autoimmune disease is diabetes, neurologic disease, or graft versus host disease.

104. The method of claim 91, wherein a therapeutically effective amount of Tregs is administered to a subject.

105. The method of claim 88, wherein the autoimmune disease is diabetes, neurologic disease, or graft versus host disease.

106. The method of claim 88, wherein a therapeutically effective amount of Tregs is administered to a subject.
